(19) United States
(12) Patent Application Publication
(10) Pub. No.: US 2007/0015152 A1
(43) Pub. Date: Jan. 18, 2007

(54) Methods for diagnosing and treating schizophrenia

(75) Inventors: Francis Paul Buxton, Winchester, MA (US); William Twitty Carpenter, Columbia, MD (US); Rosalinda Cusido Roberts, Columbia, MD (US); Carol Ann Tamminga, Dallas, TX (US)

Correspondence Address: BROWDY AND NEIMARK, P.L.L.C.;624 NINTH STREET, NW, SUITE 300, WASHINGTON, DC, 20001-5303, US

(73) Assignees: Novartis AG, Basel (CH); University of Maryland, Baltimore, MD (US)

(21) Appl. No.: 10/538,339

(22) PCT Filed: Dec. 11, 2003
(86) PCT No.: PCT/EP03/14089

§ 371 Date: Sep. 25, 2006

Related U.S. Application Data
(60) Provisional application No. 60/432,853, filed on Dec. 12, 2002.

(51) Int. Cl. C12Q 1/68

(52) U.S. Cl. 435/6

(57) ABSTRACT

The genes encoding SCYA2, GADD45B, S100A8, CDKN1A, IL1RL1, TGM2, MAFF, SERPINA3, GRO1, CD14, KIAA1075, CHI3L1, SERPINH1, MT1X, KIAA0620, TIMP1, NUMA1, DDIT3 and TOB2, are upregulated in the anterior cingulate of schizophrenic patients compared to normal patients and as such are useful drug targets for schizophrenia. Methods of screening, diagnosing and treating schizophrenia based on these genes are provided. Transgenic nonhuman animals having increased copy number or increased expression levels of these genes are also provided. The transgenic nonhuman animals are used in methods for screening for potential therapeutic agents.

### BACKGROUND

1. Field of the Invention

The present disclosure relates to genes correlated to schizophrenia and methods of using genes for diagnosis and treatment of schizophrenia.

2. Description of Related Art

Schizophrenia is a severe psychiatric disorder usually characterized by withdrawal from reality, illogical patterns of thinking, delusions and hallucinations, and accompanied in varying degrees by other emotional, behavioral, or intellectual disturbances. See Diagnostic and Statistical Manual of Mental Disorders, Fourth Edition, American Psychiatric Association, 273-315 (1994) (DSM-IV™). However, as stated therein, no single symptom is pathognomonic of schizophrenia; the diagnosis involves recognition of a constellation of signs and symptoms associated with impaired occupational or social functioning. Id. Some detectable physiological changes have been reported, e.g., neuropathological and imaging studies depicting anatomical alterations associated with the disease. Arnold et al., Acta Neuropathol. (Berl) 92, 217-231 (1996); Harrison, Brain 122, 593-624 (1999). Certain cellular aberrations have been observed and biochemical and RNA analyses have demonstrated alterations in some neurotransmitter pathways and presynaptic components. Id.; Benes, Brain Res. Rev. 31, 251-269 (2000).

At beginning stages and even at more advanced stages, schizophrenia can involve subtle behavioral changes and subtle and/or undetectable changes at the cellular and/or molecular levels in nervous system structure and function. This lack of detectable neurological defect distinguishes schizophrenia from other well-defined neurological disorders in which anatomical or biochemical pathologies are clearly manifest. Thus, there is a need for non-subjective modalities for screening and diagnosis of schizophrenia. Moreover, identification of the causative defects and the neuropathologies of schizophrenia are needed in order to enable clinicians to evaluate and prescribe appropriate courses of treatment to cure or ameliorate the symptoms of schizophrenia at early stages or when symptoms are obscured. Indeed, there are few effective therapies for the disease and its molecular basis is still not well understood.

Methods have been designed to survey alterations in mRNA expression in order to search for genes disregulated in various diseases and disorders. In organisms for which the complete genome is known, it is possible to analyze the transcripts of all genes within the cell. With other organisms, such as human, for which there is an increasing knowledge of the genome, it is possible to simultaneously monitor large numbers of genes within a cell. DNA microarray analysis is a technique that permits the quantitative measurement of the transcriptional expression of several thousand genes simultaneously. This technique permits one to generate profiles of gene expression patterns in both patients suffering from schizophrenia and control individuals. Accordingly, determination of abnormal levels of gene expression provides a signpost for therapeutic intervention.

Techniques for modifying RNA levels and activities involve ribozymes, antisense species, and RNA aptamers and small molecule promoter modulators. Ribozymes are RNAs capable of catalyzing RNA cleavage reactions, and some can be designed to specifically cleave a particular target mRNA. Ribozyme methods include exposing a cell to, inducing expression in a cell, etc. of such RNA ribozyme molecules. Activity of a target RNA (preferably mRNA) species, specifically its rate of translation, can be inhibited by the application of antisense nucleic acids. “Antisense” nucleic acids are nucleic acids capable of hybridizing to a sequence specific portion of the target RNA, e.g., its translation initiation region by virtue of some sequence that is complementary to a coding and/or non-coding region. The antisense nucleic acid can be oligonucleotides that are double-stranded or single-stranded, RNA or DNA or a modification or derivative thereof, which can be produced intracellularly by transcription of exogenous, introduced sequences in controllable quantities sufficient to perturb translation of the target RNA.

The above described techniques are emerging as an effective means for reducing the expression of specific gene products and may therefore prove to be uniquely useful in a number of therapeutic, diagnostic and research applications for the modulation of genes that are disregulated in schizophrenic patients.

We have previously discovered that three genes (decidual protein induced by progesterone (DEPP), adrenomedullin and cold shock domain protein A (cdsA)) are upregulated in schizophrenia. We have now surprisingly discovered that mRNA for nineteen other genes (disclosed in Table 1 herein) are similarly upregulated in samples from schizophrenic individuals. Thus, these genes can be used as novel drug targets for schizophrenia.

### SUMMARY

In one aspect, a method for screening for schizophrenia in a population is provided which comprises determining, in members of the population, the magnitude of expression of a gene selected from the group consisting of those disclosed in Table 1 in a sample and comparing the magnitude of expression to a baseline magnitude of expression of the gene, wherein increased gene expression indicates the presence of schizophrenia. The sample may be taken from the brain, spinal cord, lymphatic fluid, blood, urine or feces.

In another aspect, a method for diagnosing schizophrenia in a host is provided which comprises determining the magnitude of expression of a gene selected from the group consisting of those disclosed in Table 1 in a sample and comparing the magnitude of expression to a baseline magnitude of expression of the gene, wherein increased gene expression indicates the presence of schizophrenia.

In another aspect, a method for treating schizophrenia in a host is provided which comprises lowering expression of a gene selected from the group consisting of those disclosed in Table 1 by administering to the host an expression lowering amount of antisense oligonucleotide.

In another aspect, a method for treating schizophrenia in a host is provided which comprises lowering expression of a gene selected from the group consisting of the genes disclosed in Table 1 by administering to the host an expression lowering amount of a ribozyme which cleaves RNA associated with expression of the gene.

In another aspect, a method for treating schizophrenia in a host is provided which comprises lowering expression of a gene selected from the group consisting of those disclosed in Table 1 by administering one or more nucleic acid molecules designed to promote triple helix formation with said gene.

In another aspect, a method for treating schizophrenia is provided which comprises reducing the amount of a gene disclosed in Table 1 in a patient by, administering an effective amount of an antibody against the protein or proteins selected.

In another aspect, a method for treating schizophrenia is provided which comprises reducing the amount of a gene disclosed in Table 1 in a patient by administering an effective amount of a RNAi against the gene or genes selected.

In another aspect, a method of screening for compounds which are useful in the treatment of schizophrenia is provided which comprises operatively linking a reporter gene which expresses a detectable protein to a regulatory sequence for a gene selected from the group consisting of those disclosed in Table 1 to produce a reporter construct, transfecting a cell with the reporter construct, exposing the transfected cell to a test compound, and comparing the level of expression of the reporter gene after exposure to the test compound to the level of expression before exposure to the test compound, wherein a lower level of expression after exposure is indicative of a compound useful for the treatment of schizophrenia.

In another aspect, a transgenic nonhuman animal is provided whose genome stably comprises an increased copy number of a gene selected from the group consisting of those disclosed in Table 1 wherein the gene is expressed at higher than baseline levels and the animal exhibits abnormal behavior.

In another aspect, a transgenic animal is provided whose genome stably comprises a gene selected from the group consisting of those disclosed in Table 1 wherein expression of the gene is enhanced by at least one alteration in regulatory sequences of the gene such that the gene is expressed at higher than baseline levels and the animal exhibits abnormal behavior.

In another aspect, a transgenic nonhuman knockout animal is provided whose genome stably comprises a homozygous disruption in one or more genes selected from the group consisting of those disclosed in Table 1 wherein said homozygous disruption prevents the expression of the gene, and wherein said homozygous disruption results in the transgenic knockout animal exhibiting decreased expression levels of the one or more genes as compared to a wild-type animal.

In another aspect, the invention provides a method to screen for therapeutic agents that modulate symptoms of schizophrenia by administering a candidate compound to the transgenic nonhuman animals disclosed above and determining the effect of the compound on symptoms associated with schizophrenia.

### DETAILED DESCRIPTION OF PREFERRED EMBODIMENTS

In practicing the present invention, many conventional techniques in molecular biology are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

As used herein and in the appended claims, the singular forms “a”, “an”, and “the” include plural reference unless the context clearly dictates otherwise. Thus, for example, reference to the “antibody” is a reference to one or more antibodies and equivalents thereof known to those skilled in the art, and so forth.

As used herein, the definition of a “schizophrenic disease or disorder” encompasses the characterization of this disease as described in the references cited above.

“Nucleic acid sequence”, as used herein, refers to an oligonucleotide, nucleotide or polynucleotide, and fragments or portions thereof, and to DNA or RNA of genomic or synthetic origin that may be single or double stranded, and represent the sense or antisense strand.

The term “antisense” as used herein, refers to nucleotide sequences which are complementary to a specific DNA or RNA sequence. The term “antisense strand” is used in reference to a nucleic acid strand that is complementary to the “sense’ strand. Antisense molecules may be produced by any method, including synthesis by ligating the gene(s) of interest in a reverse orientation to a viral promoter which permits the synthesis of a complementary strand. Once introduced into a cell, this transcribed strand combines natural sequences produced by the cell to form duplexes. These duplexes then block either the further transcription or translation. The designation “negative” is sometimes used in reference to the antisense strand, and “positive” is sometimes used in reference to the sense strand.

As contemplated herein, antisense oligonucleotides, triple helix DNA, RNA aptamers, RNAi, ribozymes and double or single stranded RNA are directed to a nucleic acid sequence of a gene disclosed in Table 1 such that the nucleotide sequence of the gene chosen will produce gene-specific inhibition of gene expression. For example, knowledge of the target gene nucleotide sequence may be used to design an antisense molecule which gives strongest hybridization to the mRNA. Similarly, ribozymes can be synthesized to recognize specific nucleotide sequences and cleave them (Cech. J. Amer. Med Assn. 260:3030 (1988). Techniques for the design of such molecules for use in targeted inhibition of gene expression is well known to one of skill in the art.

As used herein, the term “antibody” refers to intact molecules as well as fragments thereof, such as Fa, F(ab′)₂, and Fv, which are capable of binding the epitopic determinant. Antibodies that bind polypeptides of interest can be prepared using intact polypeptides or fragments containing small peptides of interest as the immunizing antigen. The polypeptides or peptides used to immunize an animal can be derived from the translation of RNA or synthesized chemically, and can be conjugated to a carrier protein, if desired. Commonly used carriers that are chemically coupled to peptides include bovine serum albumin and thyroglobulin. The coupled peptide is then used to immunize an animal (e.g., a mouse, a rat or a rabbit).

The term “humanized antibody” as used herein, refers to antibody molecules in which amino acids have been replaced in the non-antigen binding regions in order to more closely resemble a human antibody, while still retaining the original binding ability.

A “therapeutically effective amount” is the amount of drug sufficient to treat and/or ameliorate the pathological effects of chronic pain, including but not limited to, hyperalgesia.

The term “therapeutic agent” as used herein describes any molecule, e.g. protein, carbohydrate, metal or organic compound, with the capability of affecting the molecular and clinical phenomena associated with schizophrenia. Generally a plurality of assay mixtures may be run in parallel with different agent concentrations to obtain a differential response to the various concentrations. Typically, one of these concentrations serves as a negative control, i.e. at zero concentration or below the level of detection.

“Subject” refers to any human or nonhuman organism.

The present disclosure is based on the surprising discovery that nineteen genes are associated with schizophrenia in affected individuals. More particularly, these genes are upregulated in the anterior cingulate of schizophrenic patients as compared to normal patients. The complete list of these genes is disclosed below in Table 1.

TABLE 1

| | ABBREVIATION |
|---|---|
| GENE NAME | USED HEREIN |
| Small inducible cytokineA2 | SCYA2 |
| Growth arrest and DNA-damage-inducible beta | GADD45B |
| S100 calcium binding protein A8 | S100A8 |
| Cyclin-dependent kinase inhibitor 1A p21/Cip1 | CDKN1A |
| Interleukin 1 receptor-like 1 | IL1RL1 |
| Transglutaminase | TGM2 |
| V-maf musculo aponeurotic fibrosarcoma | MAFF |
| oncogene homolog F | |
| Serine or cysteine proteinase inhibitor clade A | SERPINA3 |
| member 3 | |
| GRO1 oncogene melanoma growth stimulating | GRO1 |
| activityalpha | |
| CD14 antigen | CD14 |
| Tensin 2 | KIAA1075 |
| Chitinase 3-like 1, cartilage glycoprotein-39 | CHI3L1 |
| Serine or cysteine proteinase inhibitor clade H | SERPINH1 |
| Metallothionein 1X | MT1X |
| KIAA0620 protein | KIAA0620 |
| Tissue inhibitor of metalloproteinase 1 | TIMP1 |
| Nuclear mitotic apparatus protein 1 | NUMA1 |
| DNA-damage-inducibletranscript 3 | DDIT3 |
| Transducer of ERBB2 | TOB2 |

Accordingly, methods for the diagnosis, screening and evaluation of schizophrenia are provided in accordance with the present invention. For example, assays for determination of increased levels of expression of these genes are provided. Moreover, nucleic acid molecules encoding these genes can be used as diagnostic hybridization probes or used to design primers for diagnostic PCR analysis for the identification of gene mutations, allelic variations and regulatory defects in these genes. As used herein, “diagnosis” is intended to generally apply to individuals while “screening” is generally applicable to populations or individuals. The invention also encompasses antibodies to the products of the genes disclosed in Table 1 that can be used to decrease available plasma levels of these proteins, as well as nucleotide sequences that can be used to inhibit gene expression (e.g., antisense, RNAi and ribozyme molecules), and gene or regulatory sequence binding or replacement constructs designed to reduce or enhance gene expression (e.g., triple helix forming moieties or expression constructs that place the genes under the control of a strong promoter system).

The surprising expression characteristics of the genes disclosed in Table 1 were uncovered by examination of post mortem anterior cingulate samples from schizophrenic and normal subjects. Samples possessing high quality RNA were utilized for further study. Those skilled in the art are familiar with techniques which may be utilized to determine expression levels. For example, reverse transcriptase assays or DNA microarray analysis can be performed utilizing gene chip technology. Differentially expressed genes can be identified using a number of methods developed in accordance with established principles. Statistical significance of the expression differences between groups of samples may be determined utilizing the t-test, ANOVA or non-parametric tests. In accordance with the present invention, some genes were found to be upregulated in schizophrenic patients while others were found to be downregulated compared to baseline or normal levels. The terms “normal” and “baseline” are used interchangeably herein. Baseline levels are defined using conventional statistical techniques in connection with an analysis of a general population of non-schizophrenics. See, e.g., Example 1 herein. It should be understood, in general, that methods not otherwise specified herein are conducted in accordance with generally accepted principles known to those skilled in the art.

Quantitative rtPCR (Q-PCR) may be conducted on the same samples used for the expression level analysis described above. After conversion of RNA to cDNA using reverse transcriptase, although any conventional PCR technique can be utilized, a preferred technique may be based on the TaqMan® technique (Perkin Elmer Corp., Foster City, Calif.). In conventional PCR assays, oligonucleotide primers are designed complementary to the 5′ and 3′ends of a DNA sequence of interest. During thermal cycling, DNA is heat denatured. The sample is then brought to annealing and extension temperatures in which the primers bind their specific complements and are extended by the addition of nucleotide tri-phosphates by Taq polymerase. With repeated thermal cycling, the amount of template DNA is amplified. The presence of a dye, such as SybrGreen™, that fluoresces strongly when bound to DNA, allows the real time monitoring of total amount of DNA product in the tube. By measuring this signal, the amplified product can be quantified. The threshold cycle (C_{T}) at which the fluorescent signal is measurably different from the background noise is an accurate measure of the starting amount of cDNA in the tube and hence RNA in the sample. This method allows the quantitation of genes in a complex RNA by targeting specific DNAs. Of the genes initially identified by microarray analysis to be differentially expressed in schizophrenic patients, twenty two, decidual protein induced by progesterone (DEPP), csdA, adrenomedullin as well as those disclosed herein in Table 1, were shown to be differentially regulated in the original set of RNA samples.

In one aspect, a method of screening for schizophrenia in a population is provided which includes determining, in members of the population, the magnitude of expression of a gene selected from those disclosed in Table 1 in a sample and comparing the magnitude of expression to a baseline magnitude of expression of the gene, wherein increased gene expression indicates the presence of schizophrenia.

In another aspect, a method for diagnosing schizophrenia in a host is provided which includes determining the magnitude of expression of a gene consisting of those disclosed in Table 1 in a sample and comparing the magnitude of expression to a baseline magnitude of expression of the gene, wherein increased gene expression indicates the presence of schizophrenia. In either of the above screening or diagnosing aspects, the sample may be taken, for example, from the brain, spinal cord, lymphatic fluid, blood, urine or feces.

There are numerous techniques known to those with skill in the art to measure gene expression in a sample. For example, RNA from a cell type or tissue known, or suspected, to express a gene disclosed in Table 1, such as brain, may be isolated and tested utilizing hybridization or PCR techniques such as are described above. The isolated RNA can be derived directly from a biological sample from a patient.

In one embodiment of such a detection scheme, a cDNA molecule is synthesized from an RNA molecule of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the genes disclosed in Table 1. Those skilled in the art are familiar with techniques for designing and obtaining suitable primers. See, e.g., Table 2 in Example 2 below. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Additionally, it is possible to perform such gene expression assays “in situ”, i.e., directly upon tissue sections (fixed and/or frozen) of patient tissue obtained from biopsies or resections, such that no nucleic acid purification is necessary. Nucleic acid reagents such as those described above may be used as probes and/or primers for such in situ procedures. Alternatively, if a sufficient quantity of the appropriate cells can be obtained, standard Northern analysis can be performed to determine the level of mRNA expression of a gene disclosed in Table 1.

Regardless of the method used to quantify the expression of a gene or genes disclosed in Table 1, the level of expression in a subject of undefined etiology is compared to a known normal expression level. If the expression level of one, or more than one, of these genes is elevated above the normal or baseline level by about 25%, a diagnosis of schizophrenia may be made or confirmed. Determination of higher levels may be indicative of the severity of the disease.

As demonstrated by the Examples below, one technique for establishing baseline levels may involve real time quantitative PCR. Those skilled in the art are familiar with numerous techniques which may be utilized to test sample populations to obtain statistically sound results. For example, in carrying out this technique, a sample from a population of normal individuals is selected. The sample should be sufficiently diverse in terms of age, sex, social status, geographical distribution, previous drug and medical histories, etc. and of sufficient size to provide a meaningful statistical value. Thus, expression of a gene disclosed in Table 1 is measured in the sample of interest which defines distribution in the normal population. Baseline levels are then assigned. A set of diseased subjects is also assayed to determine validity of the test by comparing results of the diseased sample to those of the normal sample.

In accordance with the present invention, symptoms of schizophrenia associated with upregulation of a gene or genes disclosed in Table 1 may be ameliorated by decreasing the level of any one or more of these genes or gene product activity by using appropriately designed gene sequences in conjunction with well-known antisense, gene “knock-out,” ribozyme, RNAi and/or triple helix methods to decrease the level of expression of any one or more genes disclosed in Table 1.

Among the compounds that may exhibit the ability to modulate the activity, expression or synthesis of genes disclosed in Table 1 including the ability to ameliorate the symptoms of schizophrenia associated with overexpression of any one or more of these genes, are antisense, ribozyme, RNAi and triple helix molecules. Such molecules may be designed to reduce or inhibit either unimpaired, or if appropriate, mutant target gene activity. Techniques for the production and use of such molecules are well known to those skilled in the art.

Antisense RNA and DNA molecules act to block the translation of mRNA by hybridizing to target mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to coding or non-coding regions of a gene disclosed in Table 1 could be used in an antisense approach to inhibit translation of the endogenous mRNA for any one or more of these genes. mRNA. Based upon the sequences presented herein, or upon allelic or homologous genomic and/or DNA sequences, one of skill in the art can easily choose and synthesize any of a number of appropriate antisense molecules for use in accordance with the present invention. Antisense nucleic acids should be at least six nucleotides in length, and are preferably oligonucleotides ranging from 6 to about 50 nucleotides in length. In certain preferred aspects the oligonucleotide length is from about 8 to about 30 nucleotides.

Suitable antisense oligonucleotides herein encompass modified oligonucleotides which may exhibit enhanced stability, targeting or which otherwise exhibit enhanced therapeutic effectiveness. Examples of modified oligonucleotides include those where (1) at least two nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Examples of synthetic internucleoside linkages are phosphorothioates, alkylphosphonates, phosphorodithioates, phosphate esters, alkylphosphonothioates, phosphoramidates, carbamates, phosphate triesters, acetamidates, peptides, and carboxymethyl esters. Modified oligonucleotides may also have covalently modified bases and/or sugars. For example, oligonucleotides having backbone sugars which are covalently attached to low molecular weight organic groups other than a hydroxyl group at the 3′ position and other than a phosphate group at the 5′ position. Thus modified oligonucleotides may include a 2′-0-alkylated ribose group. In addition, modified oligonucleotides may include sugars such as arabinose instead of ribose. Modified oligonucleotides also can include base analogs such as C-5 propyne modified bases.

Antisense oligonucleotides may be synthesized by standard techniques known in the art, e.g., by use of an automated DNA synthesizer (such as are commercially available from Biosearch, Applied Biosystems, etc.). As examples, phosphorothioate oligonucleotides may be synthesized by the method of Stein, et al. (1988, Nucl. Acids Res. 16, 3209), methylphosphonate oligonucleotides can be prepared by use of controlled pore glass polymer supports (Sarin, et al., 1988, Proc. Natl. Acad. Sci. U.S.A. 85, 7448-7451), etc.

While antisense nucleotides complementary to the target gene coding region sequence could be used, those complementary to the transcribed, untranslated region are most preferred. A preferred site is the region encompassing the translation initiation or termination codon of the open reading frame (ORF) of the gene. Those with skill in the art are well aware of various suitable initiation or termination codons in both eukaryotes and prokaryotes.

Antisense molecules may be delivered to cells that express the target gene in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically. A preferred technique involves constructing a vector which incorporates a strong promoter to provide high expression and good yield of antisense oligonucleotides at the target site. The use of such a construct to transfect target cells in the patient results in the transcription of sufficient amounts of single stranded RNAs that will form complementary base pairs with the endogenous target gene transcripts and thereby prevent translation of the target gene mRNA. For example, a vector can be introduced such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods known to those in the art. Vectors can be, e.g., plasmid, viral, or others typically used for replication and expression in mammalian cells. It should be understood that expression of the sequence encoding the antisense RNA can be by any promoter known in the art to act in mammalian, preferably human cells. Such promoters can be inducible or constitutive. Any type of plasmid, cosmid, YAC, BAC or viral vector can be used to prepare the recombinant DNA construct which can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent or reduce translation of mRNA of any one or more genes diclosed in Table 1 herein and, therefore, expression of target gene product. (See, e.g., PCT International Publication W090/11364, published Oct. 4, 1990; Sarver, et: al., 1990, Science 247, 1222-1225). Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, incorporated herein by reference.

Ribozymes that cleave mRNA at site specific recognition sequences can be used to destroy target gene mRNAs. For example, hammerhead ribozymes may be utilized to cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art. Preferably, the ribozyme is engineered so that the cleavage recognition site is located near the 5′ end of the target gene mRNA, i.e., to increase efficiency and minimize the intracellular accumulation of non-functional protein fragments. Suitable ribozymes also include RNA endoribonucleases such as the one that occurs naturally in Tetrahymena thermophila (known as the IVS, or L-19 IVS RNA). This type of ribozymes have an eight base pair active site which hybridizes to a target RNA sequence to effect cleavage of the target RNA.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive promoter, so that transfected cells will produce sufficient quantities of the ribozyme to destroy endogenous gene messages and inhibit translation. Because ribozymes, unlike antisense molecules, are catalytic, a lower intracellular concentration is required for efficiency.

Alternatively, endogenous expression of any one of more genes disclosed in Table 1 can be reduced by targeting deoxyribonucleotide sequences complementary to the regulatory region of the target genes (i.e., the target gene promoter and/or enhancers) to form triple helical structures that prevent transcription of the target gene in target cells in the body. Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

RNA aptamers can also be introduced into or expressed in a cell to modify RNA abundance or activity. RNA aptamers are specific RNA ligands for proteins, such as for Tat and Rev RNA (Good et al., 1997, Gene Therapy 4: 45-54) that can specifically inhibit their translation. In addition, gene specific inhibition of gene expression may also be achieved using conventional double or single stranded RNA technologies. A description of such technology may be found in WO 99/32619 which is hereby incorporated by reference in its entirety. In addition, siRNA technology has also proven useful as a means to inhibit gene expression (Cullen, B R Nat. Immunol. 2002Jul;3(7):597-9; J Martinez et al., Cell 2000Sep. 6;110(5):563).

Anti-sense RNA and DNA, ribozyme, RNAi, RNA aptamer and triple helix molecules described herein may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

A method of modulating the activity of a protein encoded by a gene disclosed in Table 1 to treat schizophrenia is provided comprising exposing neutralizing antibodies to said proteins. By providing for controlled exposure to such antibodies, protein abundances/activities can be controllably modified. For example, antibodies to suitable epitopes on protein surfaces may decrease the abundance, and thereby indirectly decrease the activity, of the wild-type active form of a protein encoded by a gene disclosed in Table 1 by aggregating active forms into complexes with less or minimal activity as compared to the wild-type unaggregated wild-type form. Alternatively, antibodies may directly decrease protein activity by, e.g., interacting directly with active sites or by blocking access of substrates to active site. In either case, antibodies can be raised against specific protein species and their effects screened. The effects of the antibodies can be assayed and suitable antibodies selected that lower the target protein species concentration and/or activity. Such assays involve introducing antibodies into a cell or surrounding media, and assaying the concentration of the wild-type amount or activities of the target protein by standard means (such as immunoassays) known in the art. The net activity of the wild-type form can be assayed by assay means appropriate to the known activity of the target protein.

Antibodies can be introduced into cells in numerous ways, including, for example, microinjection of antibodies into a cell (Morgan et al., 1988, Immunology Today 9:84-86) or transforming hybridoma mRNA encoding a desired antibody into a cell (Burke et al., 1984, Cell 36:847-858). In a further technique, recombinant antibodies can be engineered and ectopically expressed in a wide variety of non-lymphoid cell types to bind to target proteins as well as to block target protein activities. Preferably, expression of the antibody is under control of a controllable promoter, such as the Tet promoter. A first step is the selection of a particular monoclonal antibody with appropriate specificity to the target protein. Then sequences encoding the variable regions of the selected antibody can be cloned into various engineered antibody formats, including, for example, whole antibody, Fab fragments, Fv fragments, single chain Fv fragments (VH and VL regions united by a peptide linker) (“ScFv” fragments), diabodies (two associated ScFv fragments with different specificities), and so forth. Intracellularly expressed antibodies of the various formats can be targeted into cellular compartments by expressing them as fusions with the various known intracellular leader sequences.

Methods for the production of antibodies capable of specifically recognizing one or more Table 1 gene product epitopes or or epitopes of conserved variants or peptide fragments of the proteins encoded by the genes disclosed in Table 1 are well known in the art. Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

Such antibodies may also be used, for example, in the detection of a Table 1 gene product in an biological sample and may, therefore, be utilized as part of a diagnostic or prognostic technique whereby patients may be tested for abnormal levels of any one or more of said gene products, and/or for the presence of abnormal forms of such gene products. Such antibodies may also be utilized in conjunction with, for example, compound screening schemes, for the evaluation of the effect of test compounds on any one or more of said gene product levels and/or activity.

For the production of antibodies against any one or more of the gene products disclosed herein various host animals may be immunized by injection with a Table 1 gene product, or a portion thereof. Such host animals may include, but are not limited to, rabbits, mice, goats, chickens and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a Table 1 gene product, or an antigenic functional derivative thereof. For the production of polyclonal antibodies, host animals such as these described above, may be immunized by injection with a Table 1 gene product supplemented with adjuvants as described above.

Monoclonal antibodies, which are homogeneous populations of antibodies to a particular antigen, may be obtained by any technique that provides for the production of antibody molecules by continuous cell lines in culture. These include, but are not limited to, the hybridoma technique of Kohler and Milstein, (1975, Nature 256, 495-497; and U.S. Pat. No. 4,376,110), the human B-cell hybridoma technique (Kosbor et al., 1983, Immunology Today 4, 72; Cole et al., 1983, Proc. Natl. Acad. Sci. USA 80, 2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb may be cultivated in vitro or in vivo.

In addition, techniques developed for the production of “chimeric antibodies” (Morrison, et al., 1984, Proc. Natl. Acad. Sci., 81, 6851-6855; Neuberger, et al., 1984, Nature 312, 604-608; Takeda, et al., 1985, Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. Techniques have also been developed for the production of humanized antibodies. (See, e.g., Queen, U.S. Pat. No. 5,585,089,). An immunoglobulin light or heavy chain variable region consists of a “framework” region interrupted by three hypervariable regions, referred to as complementarity determining regions (CDRs). The extent of the framework region and CDRs have been precisely defined (see, e.g., “Sequences of Proteins of Immunological Interest”, Kabat, E. et al., U.S. Department of Health and Human Services (1983)). Briefly, humanized antibodies are antibody molecules from non-human species having one or more CDRs from the non-human species and a framework region from a human immunoglobulin molecule. Alternatively, techniques described for the production of single chain antibodies (U.S. Pat. No. 4,946,778; Bird, 1988, Science 242, 423-426; Huston, et al., 1988, Proc. Natl. Acad. Sci. USA 85, 5879-5883; and Ward, et al., 1989, Nature 334, 544-546) can be adapted to produce single chain antibodies against any one or more of the proteins disclosed herein. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)₂ fragments, which can be produced by pepsin digestion of the antibody molecule and the Fab fragments, which can be generated by reducing the disulfide bridges of the F(ab′)₂ fragments. Alternatively, Fab expression libraries may be constructed (Huse, et al., 1989, Science, 246, 1275-1281) to allow rapid and easy identification of monoclonal Fab fragments with the desired specificity.

Antibodies, or fragments of antibodies, such as those described, above, may be used to quantitatively or qualitatively detect the presence of any one or more Table 1 gene product or conserved variants or peptide fragments thereof. This can be accomplished, for example, by immunofluorescence techniques employing a fluorescently labeled antibody coupled with light microscopic, flow cytometric, or fluorometric detection.

The antibodies (or fragments thereof) useful in the present invention may be employed histologically, as in immunofluorescence or immunoelectron microscopy, for in situ detection of any one or more Table 1 gene product, conserved variants or peptide fragments thereof. In situ detection may be accomplished by removing a histological specimen from a patient, and applying thereto a labeled antibody that binds to a polypeptide encoded by a gene disclosed in Table 1. The antibody (or fragment) is preferably applied by overlaying the labeled antibody (or fragment) onto a biological sample. Through the use of such a procedure, it is possible to determine not only the presence of any one or more of said polypeptide, conserved variant or peptide fragment, but also its distribution in the examined tissue. Using the present invention, those of ordinary skill will readily recognize that any of a wide variety of histological methods (such as staining procedures) can be modified in order to achieve in situ detection the product of a gene disclosed in Table 1

Immunoassays for a product of a gene disclosed in Table 1 conserved variants, or peptide fragments thereof will typically comprise incubating a sample, such as a biological fluid, a tissue extract, freshly harvested cells, or lysates of cells in the presence of a detectably labeled antibody capable of identifying said gene product, conserved variant or peptide fragments thereo, and detecting the bound antibody by any of a number of techniques well-known in the art. The biological sample may be brought in contact with and immobilized onto a solid phase support or carrier, such as nitrocellulose, that is capable of immobilizing cells, cell particles or soluble proteins. The support may then be washed with suitable buffers followed by treatment with the detectably labeled protein appropriate specific antibodies. The solid phase support may then be washed with the buffer a second time to remove unbound antibody. The amount of bound label on the solid support may then be detected by conventional means.

One of the ways in which specific antibodies can be detectably labeled is by linking the same to an enzyme, such as for use in an enzyme immunoassay (EIA). The enzyme, which is bound to the antibody, will react with an appropriate substrate, preferably a chromogenic substrate, in such a manner as to produce a chemical moiety that can be detected, for example, by spectrophotometric, fluorimetric or by visual means. Enzymes that can be used to detectably label the antibody are,well known. The detection can be accomplished by calorimetric methods that employ a chromogenic substrate for the enzyme. Detection may also be accomplished by visual comparison of the extent of enzymatic reaction of a substrate in comparison with similarly prepared standards. Detection may also be accomplished using any of a variety of other immunoassays. For example, by radioactively labeling the antibodies or antibody fragments, it is possible to detect any one or more proteins encoded by the genes disclosed in Table 1 through the use of a radioimmunoassay (RIA). The radioactive isotope can be detected by such means as the use of a gamma counter or a scintillation counter or by autoradiography. It is also possible to label the antibody with a fluorescent compound. When the fluorescently labeled antibody is exposed to light of the proper wavelength, its presence can then be detected due to fluorescence. Among the most commonly used fluorescent labeling compounds are green fluorescent protein, fluorescein isothiocyanate, rhodamine, phycoerythrin, phycocyanin, allophycocyanin, o-phthaldehyde and fluorescamine. The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling include luciferin, luciferase and aequorin.

The present invention contemplates production of animal models that have abnormal expression levels of any one or more genes disclosed in Table 1 to study the effects of increased or decreased levels of these proteins on such animals. Such animals provide test subjects for determining the effects of therapeutic or potentially therapeutic compounds on schizophrenia. Accordingly, Table 1 gene products can be expressed in transgenic animals. Animals of any species, including, but not limited to, mice, rats, rabbits, guinea pigs, pigs, mini-pigs, goats, sheep, and non-human primates, e.g., baboons, monkeys, and chimpanzees may be used to generate these transgenic animals. The term “transgenic,” as used herein, refers to animals expressing any one or more Table 1 gene sequence from a different species (e.g., mice expressing human gene sequences), as well as animals that have been genetically engineered to overexpress endogenous (i.e., same species) gene sequences or animals that have been genetically engineered to no longer express endogenous gene sequences (i.e., “knockout” animals), and their progeny.

Any technique known in the art may be used to introduce genes into animals to produce the founder lines of transgenic animals. Such techniques include, but are not limited to, pronuclear microinjection (Hoppe and Wagner, 1989, U.S. Pat. No. 4,873,191); retrovirus mediated gene transfer into germ lines (Van der Putten, et al., 1985, Proc. Natl. Acad. Sci., USA 82, 6148-6152); gene targeting in embryonic stem cells (Thompson, et al., 1989, Cell 56, 313-321); electroporation of embryos (Lo, 1983, Mol. Cell. Biol. 3, 1803-1814); and sperm-mediated gene transfer (Lavitrano et al., 1989, Cell 57, 717-723) (For a review of such techniques, see Gordon, 1989, Transgenic Animals, Intl. Rev. Cytol. 115, 171-229). Any technique known in the art may be used to produce transgenic animal clones containing a transgene, for example, nuclear transfer into enucleated oocytes of nuclei from cultured embryonic, fetal or adult cells induced to quiescence (Campbell, et al., 1996, Nature 380, 64-66; Wilmut, et al., 1997,Nature 385, 810-813).

The present invention provides for transgenic animals that carry a Table 1 transgene in all their cells, as well as animals that carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko, et al., 1992, Proc. Natl. Acad. Sci. USA 89, 6232-6236). The regulatory sequences required for such a cell-type specific activation will depend upon the particular cell type of interest, and will be apparent to those of skill in the art. When it is desired that the transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu, et al., 1994, Science 265, 103-106. The regulatory sequences required for such a cell-type specific inactivation will depend upon the particular cell type of interest, and will be apparent to those of skill in the art.

As mentioned above, transgenic knockout animals are also provided herein. In such transgenic animals expression of any one or more genes disclosed in Table 1 is undetectable or insignificant. Any technique known in the art may be used to produce such transgenic knockout animals. This may be achieved by a variety of mechanisms, e.g., alteration of any or all of the Table 1 genes by, e.g., introduction of a disruption of the appropriate coding sequences, e.g., insertion of one or more stop codons, insertion of a DNA fragment, etc., deletion of regulatory or coding sequence, substitution of stop codons for coding sequence, etc. The transgenic animals may be either homozygous or heterozygous for the alteration. A functional knock-out may also be achieved by the introduction of an anti-sense construct that blocks expression of the native genes. Knockouts also include conditional knockouts such as where alteration of the target gene occurs upon exposure of the animal to a substance that promotes target gene alteration, introduction of an enzyme that promotes recombination at the target gene site, or other method for directing the target gene alteration postnatally.

Once transgenic animals have been generated, the expression of the recombinant gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques described above and those that include but are not limited to Northern blot analysis of tissue samples obtained from the animal, in situ hybridization analysis, and RT-PCR (reverse transcriptase PCR). Samples of gene-expressing tissue, may also be evaluated immunocytochemically using antibodies specific for the transgene product of interest.

Through use of the subject transgenic animals or cells derived therefrom, one can identify ligands or substrates that modulate phenomena associated with schizophrenia, e.g., behavioral phenomena. A wide variety of assays may be used for this purpose, including behavioral studies, determination of the localization of drugs after administration and the like. Depending on the particular assay, whole animals may be used, or cells derived therefrom. Cells may be freshly isolated from an animal, or may be immortalized in culture. Cells of particular interest are derived from neural tissue.

Candidate therapeutic agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate therapeutic agents often comprise cyclical carbon or heterocyclic structures and/or aromatic or polyaromatic structures substituted with one or more of the above functional groups. Candidate therapeutic agents are also found among biomolecules including, but not limited to: peptides, saccharides, fatty acids, steroids, purines, pyrimidines, derivatives, structural analogs or combinations thereof.

As mentioned above, antibodies specific for proteins encoded by the genes disclosed in Table 1 may be used in screening immunoassays, particularly to detect the level of such gene product in a cell or sample. The number of cells in a sample will generally be at least about 10³, usually at least 10⁴ more usually at least about 10⁵. The cells may be dissociated, in the case of solid tissues, or tissue sections may be analyzed. Alternatively a lysate of the cells may be prepared. For example, detection may utilize staining of cells or histological sections, performed in accordance with conventional methods. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

A number of assays are known in the art for determining the effect of a drug on animal behavior and other phenomena associated with schizophrenia. Some examples are provided, although it will be understood by one of skill in the art that many other assays may also be used. The subject animals may be used by themselves, or in combination with control animals.

The screen using the transgenic animals of the invention can employ any phenomena associated with schizophrenia that can be readily assessed in an animal model. The screening for schizophrenia can include assessment of phenomena including, but not limited to: 1) analysis of molecular markers (e.g., levels of expression of any one or more Table gene products in brain tissue; presence/absence in brain tissue of various Table 1 gene splice variants; 2) assessment of behavioral symptoms associated with memory and learning; and 3) detection of neurodegeneration. Preferably, the screen will include control values (e.g., the level of production of a Table 1 gene product in the test animal in the absence of test compound(s)). Test substances which are considered positive, i.e., likely to be beneficial in the treatment of schizophrenia, will be those which have a substantial effect upon a schizophrenia associated phenomenon (e.g., test agents that are able to normalize erratic or abnormal behavior or that reduce the level of production of a Table 1 gene product to within the normal range).

The present invention also encompasses the use of cell-based assays or cell-lysate assays (e.g., in vitro transcription or translation assays) to screen for compounds or compositions that modulate the expression of any one or more genes disclosed in Table 1. To this end, constructs containing a reporter sequence linked to a regulatory element of a gene disclosed in Table 1 can be used in engineered cells, or in cell lysate extracts, to screen for compounds that modulate the expression of the reporter gene product at the level of transcription. For example, such assays could be used to identify compounds that modulate the expression or activity of transcription factors involved in expression of any one or more of the genes disclosed in Table 1, or to test the activity of triple helix polynucleotides. Alternatively, engineered cells or translation extracts can be used to screen for compounds (including antisense and ribozyme constructs) that modulate the translation of Table 1 gene mRNA transcripts, and therefore, affect expression of these gene products. Thus, regulatory regions such as a promoter are operatively linked to a gene encoding a reporter molecule such as green fluorescent protein (GFP), luciferase and the like, to create a reporter construct which is regulated by appropriate regulatory sequences for a gene disclosed in Table 1. The gene construct is then transfected into a desired cell such as a neuronal cell. The baseline expression levels of the reporter molecule are then calculated using conventional methods. The cell is then exposed to a test compound and the level of expression of the reporter molecule is determined and compared to the baseline levels. A compound which reduces the amount of reporter expression is a candidate for the treatment of schizophrenia. A second screening procedure may then be instituted to determine whether the compound affects the level of expression of any one or more genes disclosed in Table 1 by measuring the amount of RNA or protein from the native gene(s). Construction of neuronal cells incorporating a reporter gene for determining the effect of compounds on expression is known, e.g., see, Asselbergs et al., Nucleic Acids Res 27:1826-33(1998), incorporated herein by reference.

Antisense compounds, ribozymes, RNAi, RNA aptamers, antibodies and other geneknockout devices or modulators (collectively referred to for convenienceas the “modulators”) described herein may be admixed, encapsulated, conjugated or otherwise associated with other molecules, molecular structures or mixtures of compounds, as for example, liposomes, receptor targeted molecules, oral, rectal, topical, or other formulations, for assisting in uptake, distribution and/or absorption. Those skilled in the art are familiar with a myriad of techniques to produce such devices.

It is contemplated that the modulators may encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents. The term “pharmaceutically acceptable salts” refers to physiologically and pharmaceutically acceptable salts of the compounds of the invention: i.e., salts that retain the desired biological activity of the parent compound and do not impart undue toxicological effects thereto. Such compounds may be prepared according to conventional methods by one of skill in the art. (Berge et al., “Pharmaceutical Salts,” J. of Pharma Sci., 1977, 66, 1-19). The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides may be prepared as SATE [(S-acetyl-2-thioethyl)phosphate] derivatives according to the methods disclosed in WO 93/24510 to Gosselin et al., or in WO 94/26764 to Imbach et al.

The modulators herein can be utilized for diagnostics, therapeutics, prophylaxis and as research reagents and kits. For therapeutics, an animal, preferably a human, suspected of having a schizophrenic disease or disorder which can be treated by modulating the expression of one or more genes disclosed in Table 1, is treated by administering modulators in accordance with this invention, The modulators can be utilized in pharmaceutical compositions by adding an effective amount of one or more modulators to a suitable pharmaceutically acceptable diluent or carrier. Those skilled in the art are familiar with numerous techniques and formulations utilized to compound pharmaceutical compositions. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of liquids, powders or aerosols, including by nebulizer; intratracheal, intranasal, enteral, epidermal and transdermal), oral, sublingual, buccal or parenteral. Parenteral administration includes intravenous, intraarterial, subcutaneous, intraperitoneal or intramuscular injection or infusion; intramedullary or intracranial, e.g., intrathecal or intraventricular, administration. Oligonucleotides with at least one 2′-O-methoxyethyl modification may be useful for oral administration.

Pharmaceutical compositions for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Compositions and formulations for oral administration include powders or granules, suspensions or solutions in water or non-aqueous media, capsules, sachets, troches or tablets. Thickeners, flavoring agents, diluents, emulsifiers, dispersing aids or binders may be desirable. Compositions for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients. Pharmaceutical compositions of the present invention include, but are not limited to, solutions, emulsions, suspensions, foams and liposome-containing formulations. These compositions may be generated from a variety of components that include, but are not limited to, preformed liquids, self-emulsifying solids and self-emulsifying semisolids, according to conventional methods, by one of skill in the art.

The pharmaceutical formulations of the present invention, which may conveniently be presented in unit dosage form, may be prepared according to: conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product. Further details on techniques for formulation and administration of numerous dosage forms may be found in the latest edition of Remington's Pharmaceutical Sciences (Maack Publishing Co., Easton, Pa.). The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents that increase the solubility of the compounds to allow for the preparation of highly concentrated solutions. For topical or nasal administration, penetrants appropriate to the particular barrier to be permeated are used in the formulation. Such penetrants are generally known in the art.

Pharmaceutical compositions suitable for use in the invention include compositions wherein the modulators are contained in an effective amount to achieve the intended purpose. The determination of an effective dose is well within the capability of those skilled in the art. For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, which ameliorates, partially or completely, the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner in light of factors related to the subject that require treatment. Dosage and administration are adjusted to provide sufficient levels of the modulators to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g per kilogram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

All references cited herein are incorporated by reference in their entireties. The following examples are included for purposes of illustration and should not be construed as limiting the present invention.

### EXAMPLE 1

### DNA Microarray Analysis

Human anterior cingulate samples are obtained from 20 normal and 20 schizophrenic deceased subjects (Maryland Psychiatric Research Clinic, Baltimore, Md.). Good quality RNA was obtained from 19 normal (“N”) and 18 schizophrenic (“S”) samples.

The microarray analysis is performed essentially as follows. Briefly, 5 μg or less total RNA is used to synthesize cDNA which is then used as a template to generate biotinylated cRNA. 15 to 30 μg labeled RNA is obtained and hybridized to Affymetrix (Santa Clara, Calif.) Human Genome U95Av2 Arrays of the GeneChip® Human Genome U95 Set (HG-U95Av2 contains ≈12,000 sequences of full length genes) in accordance with the protocols found in the GeneChip® technical manual. Each sample is profiled in duplicate. After sample hybridization, microarrays are washed and scanned with a laser scanner.

The images obtained are used to generate absolute text files for analysis using Affymetrix GeneChip® Gene Expression Analysis Algorithms version 4. Differentially expressed genes between the normal and schizophrenic derived samples are ranked using a pattern recognition algorithm developed in accordance with established principles which generated a score for each gene being compared. The following three conditions are required for a score (equal to the mean fold change) to be generated: (1) t-test p-value<0.5%; (2) average fold-change>1.5; (3) maximum mean AvgDiff (expression levels on an Affymetrix chip)>200. If one or more of the above conditions is not met by a gene in comparison, the score assigned is zero. Results indicate that several genes are found to be differentially expressed in schizophrenic patients when compared to normal (see Table 2 below).

### EXAMPLE 2

### Real Time Quantitative PCR Confirmation of Differentially Regulated Genes

Probe pairs for real time quantitative PCR (Q-PCR) are designed for the 56 altered genes identified in Example 1. Affymetrix provides a file of sequences from which the probes on the chip are derived. From this file, the sequences corresponding to these 56 altered genes are obtained, and the probe pairs are prepared. Where a good pair of primers cannot be obtained from Affymetrix sequence, a longer sequence can be obtained from Ref Seq. (See Pruitt K D, Maglott D R Nucleic Acids Res 2001Jan. 1,;29 (1):137-140;Pruitt K D, et al. Trends Genet. 2000 January;16(1):44-47) with a good BLAST score against the Affymetrix sequence and the primers are designed from that sequence. The sequences of the probe pairs and the best RefSeq or Genbank hits are are presented in Table 2. Most were detected as differentially expressed in schizophrenic patients compared to normal by Affymetrix GeneChips®). ACTB and GAPD were included as controls.

TABLE 2

| | | | |
|---|---|---|---|
| | | RefSeq or | |
| Oligopair | PCR Primer Sequences | GenBank IDs | GeneName |

| | | | | |
|---|---|---|---|---|
| SZ1-29 | CACCCAGCAGAGCAG | TTTTGCTTTATTTCT | NM_003651 | Cold shock domain |
| | TGTGA | GAATGGTCATCT | | protein A (CSDA) |
| | (Seq ID No 1) | (Seq ID No 59) | | |
| SZ1-25 | GAGTCTGAAGGACCC | TCTGTCCCTTCACC | NM_007021 | Decidual protein |
| | TAGTTCCTAGA | TCTGATCA | | induced by progesterone |
| | (Seq ID No 2) | (Seq ID No 60) | | (DEPP) |
| SZ1-11 | TCGCCCACAAACTGA | ACGCATTGCACTTT | NM_001124 | Adrenomedullin (ADM) |
| | TTTCTC | TCCTCTTT | | |
| | (Seq ID No 3) | (Seq ID No 61) | | |
| JSZ9 | GTGCCTGTAGTGACT | AGGCCCCGGGTCT | NM_002673 | Plexin B1 (PLXNB1) |
| | GACAAGCA | AGGA | | |
| | (Seq ID No 4) | (Seq ID No 62) | | |
| JSZ8 | TTCTGACAACTGGTG | TTGGACCCAGACG | NM_002509 | NK2 transcription factor |
| | GCAGATT | GGAAA | | homolog B (NKX2B) |
| | (Seq ID No 5) | (Seq ID No 63) | | |
| JSZ7 | GCCTCCCAGTGCAAA | CAGGGAGAAGAAC | NM_013279 | Chromosome 11 ORF 9 |
| | TCCT | TGGGAGTTAACT | | (C11orf9) |
| | (Seq ID No 6) | (Seq ID No 64) | | |
| JSZ6 | GACCTGTTGTAATTG | ACGGCAAGGTATC | AF305057 | RTS gene (RTS) |
| | CTCCTCATGT | GACAGGAT | | |
| | (Seq ID No 7) | (Seq ID No 65) | | |
| JSZ5A | TATTAACAGGATAAC | CCTCGGCCCTGGT | NM_004636 | Immunoglobulin domain |
| | CCTTGAATGTAGCA | CGTT | | Ig secreted semaphorin3 |
| | (Seq ID No 8) | (Seq ID No 66) | | (SEMA3B) |
| JSZ57 | ACATGCCGTTGCTCA | GCCATCAACTTCAA | NM_000784 | Cytochrome P450 sub- |
| | AAGCT | TTTCCTTTTC | | family XXVIIA (CYP27A1) |
| | (Seq ID No 9) | (Seq ID No 67) | | |
| JSZ56B | CAAGCAGAAGTGGGT | TTAGCTGCAGATTC | NM_002982 | Small inducible |
| | TCAGGAT | TTGGGTTGT | | cytokineA2 (SCYA2) |
| | (Seq ID No 10) | (Seq ID No 68) | | |
| JSZ55B | GGACTCGATTCTGCC | ACAATGGGCTCGAC | NM_004123 | Gastric inhibitorypoly- |
| | CTTCA | TTAGCATAA | | peptide (GIP) |
| | (Seq ID No 11) | (Seq ID No 69) | | |
| JSZ54A | AAGGGATTCGGCCCA | CAGAGACCAAGAA | NM_001686 | ATP synthaseH+transport- |
| | ATAAT | GGTCAAGATGTACT | | ing mitochondrial F1 |
| | (Seq ID No 12) | (Seq ID No 70) | | complex beta polypeptide |
| | | | | (ATP5B) |
| JSZ53C | TCAATCCTGCATCCC | ACAGCCACCAGTGA | NM_001511 | GRO1 oncogene melanoma |
| | CCATA | GCTTCCT | | growth stimulating |
| | (Seq ID No 13) | (Seq ID No 71) | | activityalpha (GRO1) |
| JSZ52A | ATGATCCTATTCTGTG | TTCTTAAGGCTGTA | NM_001249 | Ectonucleoside triphosphate |
| | TTAGCTCCAAT | ATTTATGCACAGTT | | diphosphohydrolase 5 |
| | (Seq ID No 14) | (Seq ID No 72) | | (ENTPD5) |
| JSZ51 | GACCCACCAGTGCCT | CTCCCCACTTTGGG | NM_002391 | Midkine neurite growth- |
| | TCTGT | CACTTA | | promoting factor 2 (MDK) |
| | (Seq ID No 15) | (Seq ID No 73) | | |
| JSZ50 | CTGCCTTTTCCTGCG | GACAGAGAGCCGC | NM_000591 | CD14 antigen (CD14) |
| | AACA | CATCAGT | | |
| | (Seq ID No 16) | (Seq ID No 74) | | |
| JSZ49 | ACAAGCTCAGAGCCC | ATTCCTAAGGGAGG | NM_015319 | Tensin 2 (KIAA1075) |
| | ACATCA | GTGCTTTCT | | |
| | (Seq ID No 17) | (Seq ID No 75) | | |
| JSZ48 | AGGGCACCACGCAG | CCTGGACAAGTTTG | BC036944 | EST clone IMAGE: |
| | ACAT | AAGGACAGA | | 5395238 |
| | (Seq ID No 18) | (Seq ID No 76) | | |
| JSZ46 | TGGAGTGTCGGATCC | CTCCCACAAGAATG | NM_001277 | Choline kinase (CHK) |
| | TGTGA | ATGATGTCA | | |
| | (Seq ID No 19) | (Seq ID No 77) | | |
| JSZ45A | GCCCGCATGTCTACT | TGAAGTCAGGGACA | NM_006230 | DNA polymerase delta 2 |
| | TTTGTG | GTCACCAA | | regulatory subunit (POLD2) |
| | (Seq ID No 20) | (Seq ID No 78) | | |
| JSZ44 | TGTACGAGTCGGCCA | GATTTGCAGGGCG | NM_015675 | Growth arrest and DNA- |
| | AGTTG | ATGTCAT | | damage-induciblebeta |
| | (Seq ID No 21) | (Seq ID No 79) | | (GADD45B) |
| JSZ43A | AGGCTGAGCAAGCAG | CTCACCAACCTGCA | NM_000824 | Glycine receptor beta |
| | ATGGA | AAGTGCTA | | (GLRB) |
| | (Seq ID No 22) | (Seq ID No 80) | | |
| JSZ42A | AAGGCTATGTTTACG | TGAGCTGCCCCTCT | NM_022740 | 3′ end of homeodomain |
| | TTTTACTCATTGT | GTCTCT | | interacting protein |
| | (Seq ID No 23) | (Seq ID No 81) | | kinase 2 (HIPK2) |
| JSZ41 | TGAGGCATCGCAATG | GGGCAGGGAGTTG | NM_001276 | Chitinase 3-like 1, |
| | TAAGACT | AAGAAATT | | cartilage glycoprotein-39 |
| | (Seq ID No 24) | (Seq ID No 82) | | (CHI3L1) |
| JSZ40A | ACCTCCCCGCCGAGT | GAGGCTCCAGCTTA | NG_000006 | Genomic alpha globin |
| | TC | ACGGTATTT | | region (HBAalpha) |
| | (Seq ID No 25) | (Seq ID No 83) | | |
| JSZ4 | CCTCCGGGCGTGTGA | CCTCTTGATTTCCC | NM_139351 | Bridging integrator 1 |
| | A | TTTGCTCTT | | (BIN1) |
| | (Seq ID No 26) | (Seq ID No 84) | | |
| JSZ36 | TCTTTGGCTTCAGAAT | CAGCAAACTCAACC | NM_000794 | Dopamine receptor D1(DRD1) |
| | TGTTTTTAGA | CATCTCATT | | |
| | (Seq ID No 27) | (Seq ID No 85) | | |
| JSZ35 | GCTATAATCCCCCTC | TGGAGGATTGATCT | NM_004960 | Fusion derived from t12; |
| | AGGGCTAT | TGGCCATA | | 16 malignant liposarcoma |
| | (Seq ID No 28) | (Seq ID No 86) | | (FUS) |
| JSZ34A | GTGAATCTGCACCAA | CTAGTGAGAGGGTA | NM_004083 | DNA-damage-inducible- |
| | GCATGA | GTCAGTAGCCACTT | | transcript 3 (DDIT3) |
| | (Seq ID No 29) | (Seq ID No 87) | | |
| JSZ33 | GAGCCGGACTGGAC | CCTGACAGGATCC | NM_000918 | Pro collagen protein |
| | ATGGT | GGAAGTCT | | disulfide isomerase |
| | (Seq ID No 30) | (Seq ID No 88) | | (P4HB) |
| JSZ32C | CAATGCCCTCTTTATT | GTGGAAGGGCGGG | NM_002309 | Leukemia inhibitory |
| | CTCTATTACACA | AAGTC | | factor (LIF) |
| | (Seq ID No 31) | (Seq ID No 89) | | |
| JSZ31A | CCGAGTGTCCTCAGT | CCATCTTTATCACC | NM_002964 | S100 calcium binding |
| | ATATCAGGAA | AGAATGAGGAA | | protein A8 (S100A8) |
| | (Seq ID No 32) | (Seq ID No 90) | | |
| JSZ30 | TGCAGGCATGGTCCC | AGTCAGTTCATCTG | NM_004428 | Ephrin-A1 (EFNA1) |
| | TTAA | GGCATCCT | | |
| | (Seq ID No 33) | (Seq ID No 91) | | |
| JSZ3 | CAGCGACCTTCCTCA | AGCCTCTACTGCCA | NM_078467 | Cyclin-dependent kinase |
| | TCCA | CCATCTTAA | | inhibitor 1A p21/Cip1 |
| | (Seq ID No 34) | (Seq ID No 92) | | (CDKN1A) |
| JSZ2A | GCAGGATGGACTCTT | CAGCCAACAGTGTA | NM_003254 | Tissue inhibitor of |
| | GCACAT | GGTCTTGGT | | metalloproteinase 1 |
| | (Seq ID No 35) | (Seq ID No 93) | | (TIMP1) |
| JSZ29 | TGAACTTCATCAGTTA | CCCTTCGCCGGCTT | NM_004746 | Discs large homolog- |
| | AAGGCCAAT | CTT | | associated protein 1 |
| | (Seq ID No 36) | (Seq ID No 94) | | (DLGAP1) |
| JSZ28B | CCTCCGGGAAGTCTT | GGCCAAACGCACC | NM_000756 | Corticotropin releasing- |
| | GGAA | GTTT | | hormone (CRH) |
| | (Seq ID No 37) | (Seq ID No 95) | | |
| JSZ27B | GTGTTTGCCTCAGGC | CCAGTCCTATTGAA | NM_016232 | Interleukin 1receptor- |
| | CAACT | TGTGGGACTT | | like 1 (IL1RL1) |
| | (Seq ID No 38) | (Seq ID No 96) | | |
| JSZ26 | AGAGCCCTCCATCAC | CAGCCCTATTCCAC | AF209502 | Calpain (CAPN3) |
| | CTTCA | TGAGTTAGTTT | | |
| | (Seq ID No 39) | (Seq ID No 97) | | |
| JSZ25 | AGCACAAGAGCCTCT | TGTACACGAACTCC | NM_016272 | Transducer of ERBB22 |
| | CTCTGTCTAT | TTGGCATT | | (TOB2) |
| | (Seq ID No 40) | (Seq ID No 98) | | |
| JSZ24 | CTGGGTGAATGCCTT | ACTTTATGCTCCGA | NM_005393 | Plexin B3 (PLXNB3) |
| | GAAGAA | GGTGGTACA | | |
| | (Seq ID No 41) | (Seq ID No 99) | | |
| JSZ23 | GGTACCAGCCTTGGA | TTCCGGGCTCAGCA | NM_004353 | Serine or cysteine proteinase |
| | TACTCCAT | TCAT | | inhibitor clade H (SERPINH1) |
| | (Seq ID No 42) | (Seq ID No 100) | | |
| JSZ22 | CCTCGAAATGGACCC | GCAGCCCTGGGCA | NM_005952 | Metallothionein 1X(MT1X) |
| | CAACT | CACT | | |
| | (Seq ID No 43) | (Seq ID No 101) | | |
| JSZ21 | ACGTATCATGCACCA | TCTGGAACAGTCAT | XM_030707 | KIAA0620 protein (KIAA0620) |
| | ACTGTGAA | TTCCAGTGTT | | |
| | (Seq ID No 44) | (Seq ID No 102) | | |
| JSZ20 | AAGAAGAAGTGACCA | AGATGGGTTGTGAA | NM_006501 | Myelin-associatedoligo- |
| | AGGAGGAGTT | GCAATGAGT | | dendrocyte basic protein |
| | (Seq ID No 45) | (Seq ID No 103) | | (MOBP) |
| JSZ81 | AGAGGAGCGGCAGG | CTTGAAACTGCCCA | NM_004613 | Transglutaminase (TGM2) |
| | AGTATGT | AAATTCCA | | |
| | (Seq ID No 46) | (Seq ID No 104) | | |
| JSZ18 | CCTTCCTCTCTGCAA | GAGAACTCCTGGTG | NM_005567 | Lectingalactoside-binding- |
| | TGACCTT | GACCCTAGT | | soluble3binding protein |
| | (Seq ID No 47) | (Seq ID No 105) | | (LGALS3BP) |
| JSZ17 | GCGCCCATTGATGAG | CATCCTCCCACAGG | NM_138924 | GuanidinoacetateN-methyl- |
| | CAT | CCTTT | | transferase (GAMT) |
| | (Seq ID No 48) | (Seq ID No 106) | | |
| JSZ16 | ACCCCTGCCTTTGAT | GAGAATAACTTAGA | NM_012323 | V-maf musculo aponeurotic |
| | TGTCA | TCCGTGCAATAAAT | | fibrosarcoma oncogene |
| | (Seq ID No 49) | AA | | homolog F (MAFF) |
| | | (Seq ID No 107) | | |
| JSZ15 | CCTGCTAAGAAGCTG | GAGTGGCTTCTCAG | NM_032978 | Dystro brevin alpha (DTNA) |
| | ACTAATGCA | GCTGATCT | | |
| | (Seq ID No 50) | (Seq ID No 108) | | |
| JSZ14A | AAGCCTCAGCAGTTC | TCATAATTCTGCATT | NM_003182 | Tachykinin precursor 1: |
| | TTTGGATT | GCACTCCTT | | substance K and P, neurokinin |
| | (Seq ID No 51) | (Seq ID No 109) | | 1 and 2, neuromedin L, |
| | | | | neurokinin alpha, K and gamma |
| | | | | (TAC1) |
| JSZ13A | CCATCAAGACGGAGC | CCTTCTTCTTGCCA | NM_007367 | RNA binding protein (RALY) |
| | TGACA | TCTGGATT | | |
| | (Seq ID No 52) | (Seq ID No 110) | | |
| JSZ12A | TAAGAATGGAGCAGT | GGGACGCTGTGTC | NM_000731 | Cholecystokinin B receptor |
| | ACATGGGAAA | TCTCCAA | | (CCKBR) |
| | (Seq ID No 53) | (Seq ID No 111) | | |
| JSZ11 | GTTCAGAGAGATAGG | GGTGAAGGCTTCCT | NM_001085 | Serine or cysteine proteinase |
| | TGAGCTCTACCT | CAATGC | | inhibitor clade A member 3 |
| | (Seq ID No 54) | (Seq ID No 112) | | (SERPINA3) |
| JSZ10C | TCCTCAACACACCCA | GAGAACGGCGGGT | NM_006185 | Nuclear mitotic apparatus |
| | AGAAGCT | TCCA | | protein 1 (NUMA1) |
| | (Seq ID No 55) | (Seq ID No 113) | | |
| JSZ1 | GGAACTTTTCTATTAC | CAGAGCGGGTGGG | NM_006494 | Ets2 repressor factor (ERF) |
| | AATCGCTTAGGA | TCAGA | | |
| | (Seq ID No 56) | (Seq ID No 114) | | |
| GAPDH | ATGGGGAAGGTGAAG | TAAAAGCAGCCCTG | NM_002046 | Glyceraidehyde-3-phosphate |
| SD | GTCG | GTGACC | | dehydrogenase (GAPD) |
| | (Seq ID No 57) | (Seq ID No 115) | | |
| Actin2 | AAGGATTCCTATGTG | TCCATGTCGTCCCA | NM_001101 | beta actin (ACTB) |
| | GGCGA | GTTGGT | | |
| | (Seq ID No 58) | (Seq ID No 116) | | |

RNA levels are then measured using Q-PCR. Briefly, cDNA is synthesized using random hexamers, diluted in a master mix containing TAQ polymerase, SybrGreen™ (Molecular Probes, Inc., Eugene, Oreg.), unlabeled nucleotides, buffer and water. The mixture is aliquotted into TaqMan® plates (Perkin Elmer) and pairs of oligonucleotides are added to the appropriate wells. Each sample is assayed in at least duplicate wells and every sample is assayed with every oligonucleotide pair where the transcriptase is omitted from the first reaction (noRT controls). The threshold cycle (C_{T}) is calculated using Perkin Elmer software ABI Prism® 7700 Sequence Detection System Revison B. The C_{T} value is defined as the cycle at which a statistically significant increase in fluorescence (from the SybrGreen™) is detected. A lower C_{T} value is indicative of a higher mRNA concentration.

cDNA is separately prepared from a subset of 16 N (normal) and 16 S (schizophrenic) samples according to conventional methods. Yield is estimated using PicoGreen™ (Molecular Probes, Inc., Eugene, Oreg.) assays. All the genes were measured by Q-PCR run on the individual cDNA samples. The individual C_{T} values for these genes relative to the actin level are examined and t-test and Kruskal Wallace p-values are calculated to test the null hypothesis that the two samples N and S are derived from the same population. Data indicate that thirteen genes are found to be differentially expressed between all the normal and schizophrenic anterior cingulate samples. These genes are listed in Table 3

TABLE 3
Genes upregulated in all schizophrenics relative to all the normals

| | RefSeq or | |
|---|---|---|
| Oligopair | GenBank IDs | GeneName |
| SZ1-29 | NM_003651 | Cold shock domain protein A (CSDA)* |
| SZ1-25 | NM_007021 | Decidual protein induced by progesterone |
| | | (DEPP)* |
| SZ1-11 | NM_001124 | Adrenomedullin (ADM)* |
| JSZ56B | NM_002982 | Small inducible cytokineA2 (SCYA2) |
| JSZ44 | NM_015675 | Growth arrest and DNA-damage- |
| | | induciblebeta (GADD45B) |
| JSZ34A | NM_004083 | DNA-damage-inducibletranscript 3 (DDIT3) |
| JSZ31A | NM_002964 | S100 calcium binding protein A8 (S100A8) |
| JSZ3 | NM_078467 | Cyclin-dependent kinase inhibitor 1A |
| | | p21/Cip1 (CDKN1A) |
| JSZ27B | NM_016232 | Interleukin 1 receptor-like 1 (IL1RL1) |
| JSZ25 | NM_016272 | Transducer of ERBB2 (TOB2) |
| JSZ81 | NM_004613 | Transglutaminase (TGM2) |
| JSZ16 | NM_012323 | V-maf musculo aponeurotic fibrosarcoma |
| | | oncogene homolog F (MAFF) |
| JSZ11 | NM_001085 | Serine or cysteine proteinase inhibitor |
| | | clade A member 3 (SERPINA3) |

*Interestingly, three genes that we identified previously as being associated with schizophrenia (decidual protein induced by progesterone (DEPP), adrenomedullin and cold shock domain protein A (cdsA)) are detected in these experiments, confirming the validity of the data disclosed herein.

Hierarchical clustering of the Q-PCR data showed that of the 16 S samples, seven formed a tight cluster. This indicates that based on expression levels of these 56 measured genes, these seven schizophrenic patients are more similar to one another than to any of the other patients or the normal controls. As such, the seven schizophrenic patients may define a subset of the disease, particularly since when these seven patients were compared to the rest of the other S and N patients, the above set of 13 genes as well as a further 9 genes, were significantly differentially regulated. These additional genes are listed in Table 4

TABLE 4
Genes upregulated in 7 schizophrenics

| | RefSeq or | |
|---|---|---|
| Oligopair | GenBank IDs | GeneName |
| JSZ53C | NM_001511 | GRO1 oncogene melanoma growth |
| | | stimulating activity alpha (GRO1) |
| JSZ50 | NM_000591 | CD14 antigen (CD14) |
| JSZ49 | NM_015319 | Tensin 2 (KIAA1075) |
| JSZ41 | NM_001276 | Chitinase 3-like 1, cartilage |
| | | glycoprotein-39 (CHI3L1) |
| JSZ23 | NM_004353 | Serine or cysteine proteinase inhibitor |
| | | clade H (SERPINH1) |
| JSZ22 | NM_005952 | Metallothionein 1X(MT1X) |
| JSZ21 | XM_030707 | KIAA0620 protein (KIAA0620) |
| JSZ2A | NM_003254 | Tissue inhibitor of metalloproteinase 1 |
| | | (TIMP1) |
| JSZ10C | NM_006185 | Nuclear mitotic apparatus protein 1 |
| | | (NUMA1) |

cDNA sequences

| | |
|---|---|
| NM_002982: Small inducible cytokineA2 (SCYA2) | |
| GGAACCGAGAGGCTGAGACTAACCCAGAAACATCCAATTCTCAAACTGAAGCTCGCACTCTCGC | Seq. ID No. 117 |
| CTCCAGCATGAAAGTCTCTGCCGCCCTTCTGTGCCTGCTGCTCATAGCAGCCACCTTCATTCCCC | |
| AAGGGCTCGCTCAGCCAGATGCAATCAATGCCCCAGTCACCTGCTGTTATAACTTCACCAATAGG | |
| AAGATCTCAGTGCAGAGGCTCGCGAGCTATAGAAGAATCACCAGCAGCAAGTGTCCCAAAGAAG | |
| CTGTGATCTTCAAGACCATTGTGGCCAAGGAGATCTGTGCTGACCCCAAGCAGAAGTGGGTTCA | |
| GGATTCCATGGACCACCTGGACAAGCAAACCCAAACTCCGAAGACTTGAACACTCACTCCACAAC | |
| CCAAGAATCTGCAGCTAACTTATTTTCCCCTAGCTTTCCCCAGACACCCTGTTTTATTTTATTATAA | |
| TGAATTTTGTTTGTTGATGTGAAACATTATGCCTTAAGTAATGTTAATTCTTATTTAAGTTATTGATG | |
| TTTTAAGTTTATCTTTCATGGTACTAGTGTTTTTTAGATACAGAGACTTGGGGAAATTGCTTTTCCT | |
| CTTGAACCACAGTTCTACCCCTGGGATGTTTTGAGGGTCTTTGCAAGAATCATTAATACAAAGAAT | |
| TTTTTTTAACATTCCAATGCATTGCTAAAATATTATTGTGGAAATGAATATTTTGTAACTATTACACC | |
| AAATAAATATATTTTTGTACAAAAAAAAAAAAAA | |
| NM_015675: Growth arrest and DNA-damage-induciblebeta (GADD45B) | |
| CTAGCTCTGTGGGAAGGTTTTGGGCTCTCTGGCTCGGATTTTGCAATTTCTCCCTGGGGACTGCC | Seq ID No. 118 |
| GTGGAGCCGCATCCACTGTGGATTATAATTGCAACATGACGCTGGAAGAGCTCGTGGCGTGCGA | |
| CAACGCGGCGCAGAAGATGCAGACGGTGACCGCCGCGGTGGAGGAGCTTTTGGTGGCCGCTCA | |
| GCGCCAGGATCGCCTCACAGTGGGGGTGTACGAGTCGGCCAAGTTGATGAATGTGGACCCAGA | |
| CAGCGTGGTCCTCTGCCTCTTGGCCATTGACGAGGAGGAGGAGGATGACATCGCCCTGCAAATC | |
| CACTTCACGCTCATCCAGTCCTTCTGCTGTGACAACGACATCAACATCGTGCGGGTGTCGGGCAA | |
| TGCGCGCCTGGCGCAGCTCCTGGGAGAGCCGGCCGAGACCCAGGGCACCACCGAGGCCCGAG | |
| ACCTCCACTGTCTTCCCTTCCTACAGAACCCTCACACGGACGCCTGGAAGAGCCACGGCTTGGT | |
| GGAGGTGGCCAGCTACTGCGAAGAAAGCCGGGGCAACAACCAGTGGGTCCCCTACATCTCTCTT | |
| CAGGAACGCTGAGGCCCTTCCCAGCAGCAGAATCTGTTGAGTTGCTGCCAACAAACAAAAAATAC | |
| AATAAATATTTGAACCCCCTCCCCCCCAGCACAACCCCCCCAAAACAACCCAACCCACGAGGACC | |
| ATCGGGGGCAGGTCGTTGGAGACTGAAGAGAAAGAGAGAGAGGAGAAGGGAGTGAGGGGCCG | |
| CTGCCGCCTTCCCCATCACGGAGGGTCCAGACTGTCCACTCGGGGGTGGAGTGAGACTGACTG | |
| CAAGCCCCACCCTCCTTGAGACTGGAGCTGAGCGTCTGCATACGAGAGACTTGGTTGAAACTTG | |
| GTTGGTCCTGTCTGCACCCTCGACAAGACCACACTTTGGGACTTGGGAGCTGGGGCTGAAGTT | |
| GCTCTGTACCCATGAACTCCCAGTTTGCGAATTAATAAGAGACAATCTATTTTGTTACTTGCACTT | |
| GTTATTCGAACCACTGAGAGCGAGATGGGAAGCATAGATATCTATATTTTTATTTCTACTATGAGG | |
| GCCTTGTAATAAATTTCTAAAGCCTCAAAAAA | |
| NM_002964: S100 calcium binding protein A8 (S100A8) | |
| ATGTCTCTTGTCAGCTGTCTTTCAGAAGACCTGGTGGGGCAAGTCCGTGGGCATCATGTTGACCG | Seq. ID No. 119 |
| AGCTGGAGAAAGCCTTGAACTCTATCATCGACGTCTACCACAAGTACTCCCTGATAAAGGGGAAT | |
| TTCCATGCCGTCTACAGGGATGACCTGAAGAAATTGCTAGAGACCGAGTGTCCTCAGTATATCAG | |
| GAAAAAGGGTGCAGACGTCTGGTTCAAAGAGTTGGATATCAACACTGATGGTGCAGTTAACTTCC | |
| AGGAGTTCCTCATTCTGGTGATAAAGATGGGCGTGGCAGCCCACAAAAAAAGCCATGAAGAAAG | |
| CCACAAAGAGTAGCTGAGTTACTGGGCCCAGAGGCTGGGCCCCTGGACATGTACCTGCAGAATA | |
| ATAAAGTCATCAATACCTCAAAAAAAAAAAAAAAAAAAAA | |
| NM_078467: Cyclin-dependent kinase inhibitor 1A p21/Cip1 (CDKN1A) | |
| AGCTGAGGTGTGAGCAGCTGCCGAAGTCAGTTCCTTGTGGAGCCGGAGCTGGGCGCGGATTCG | Seq. ID No. 120 |
| CCGAGGCACCGAGGCACTCAGAGGAGGTGAGAGAGCGGCGGCAGACAACAGGGGACCCCGGG | |
| CCGGCGGCCCAGAGCCGAGCCAAGCGTGCCCGCGTGTGTCCCTGCGTGTCCGCGAGGATGCG | |
| TGTTCGCGGGTGTGTGCTGCGTTCACAGGTGTTTCTGCGGCAGGCGCCATGTCAGAACCGGCTG | |
| GGGATGTCCGTCAGAACCCATGCGGCAGCAAGGCCTGCCGCCGCCTCTTCGGCCCAGTGGACA | |
| GCGAGCAGCTGAGCCGCGACTGTGATGCGCTAATGGCGGGCTGCATCCAGGAGGCCCGTGAGC | |
| GATGGAACTTCGACTTTGTCACCGAGACACCACTGGAGGGTGACTTCGCCTGGGAGCGTGTGCG | |
| GGGCCTTGGCCTGCCCAAGCTCTACCTTCCCACGGGGCCCCGGCGAGGCCGGGATGAGTTGGG | |
| AGGAGGCAGGCGGCCTGGCACCTCACCTGCTCTGCTGCAGGGGACAGCAGAGGAAGACCATGT | |
| GGACCTGTCACTGTCTTGTACCCTTGTGCCTCGCTCAGGGGAGCAGGCTGAAGGGTCCCCAGGT | |
| GGACCTGGAGACTCTCAGGGTCGAAAACGGCGGCAGACCAGCATGACAGATTTCTACCACTCCA | |
| AACGCCGGCTGATCTTCTCCAAGAGGAAGCCCTAATCCGCCCACAGGAAGCCTGCAGTCCTGGA | |
| AGCGCGAGGGCCTCAAAGGCCCGCTCTACATCTTCTGCCTTAGTCTCAGTTTGTGTGTCTTAATT | |
| ATTATTTGTGTTTTAATTTAAACACCTCCTCATGTACATACCCTGGCCGCCCCCTGCCCCCCAGCC | |
| TCTGGCATTAGAATTATTTAAACAAAAACTAGGCGGTTGAATGAGAGGTTCCTAAGAGTGCTGGG | |
| CATTTTTATTTTATGAAATACTATTTAAAGCCTCCTCATCCCGTGTTCTCCTTTTCCTCTCTCCCGG | |
| AGGTTGGGTGGGCCGGCTTCATGCCAGCTACTTCCTCCTCCCCACTTGTCCGCTGGGTGGTACC | |
| CTCTGGAGGGGTGTGGCTCCTTCCCATCGCTGTCACAGGCGGTTATGAAATTCACCCCCTTTCCT | |
| GGACACTCAGACCTGAATTCTTTTTCATTTGAGAAGTAAACAGATGGCACTTTGAAGGGGCCTCA | |
| CCGAGTGGGGGCATCATCAAAAACTTTGGAGTCCCCTCACCTCCTCTAAGGTTGGGCAGGGTGA | |
| CCCTGAAGTGAGCACAGCCTAGGGCTGAGCTGGGGACCTGGTACCCTCCTGGCTCTTGATACCC | |
| CCCTCTGTCTTGTGAAGGCAGGGGGAAGGTGGGGTCCTGGAGCAGACCACCCCGCCTGCCCTC | |
| ATGGCCCCTCTGACCTGCACTGGGGAGCCCGTCTCAGTGTTGAGCCTTTTCCCTCTTTGGCTCC | |
| CCTGTACCTTTTGAGGAGCCCCAGCTACCCTTCTTCTCCAGCTGGGCTCTGCAATTCCCCTCTGC | |
| TGCTGTCCCTCCCCCTTGTCCTTTCCCTTCAGTACCCTCTCAGCTCCAGGTGGCTCTGAGGTGCC | |
| TGTCCCACCCCCACCCCCAGCTCAATGGACTGGAAGGGGAAGGGACACACAAGAAGAAGGGCA | |
| CCCTAGTTCTACCTCAGGCAGCTCAAGCAGCGACCGCCCCCTCCTCTAGCTGTGGGGGTGAGG | |
| GTCCCATGTGGTGGCACAGGCCCCCTTGAGTGGGGTTATCTCTGTGTTAGGGGTATATGATGGG | |
| GGAGTAGATCTTTCTAGGAGGGAGACACTGGCCCCTCAAATCGTCCAGCGACCTTCCTCATCCA | |
| CCCCATCCCTCCCCAGTTCATTGCACTTTGATTAGCAGCGGAACAAGGAGTCAGACATTTTAAGA | |
| TGGTGGCAGTAGAGGCTATGGACAGGGCATGCCACGTGGGCTCATATGGGGCTGGGAGTAGTT | |
| GTCTTTCCTGGCACTAACGTTGAGCCCCTGGAGGCACTGAAGTGCTTAGTGTACTTGGAGTATTG | |
| GGGTCTGACCCCAAACACCTTCCAGCTCCTGTAACATACTGGCCTGGACTGTTTTCTCTCGGCTC | |
| CCCATGTGTCCTGGTTCCCGTTTCTCCACCTAGACTGTAAACCTCTCGAGGGCAGGGACCACAC | |
| CCTGTACTGTTCTGTGTCTTTCACAGCTCCTCCCACAATGCTGAATATACAGCAGGTGCTCAATAA | |
| ATGATTCTTAGTGACTTTAAAAAAAAAAAAAAAAAAAA | |
| NM_016232: Interleukin 1receptor-like 1 (IL1RL1) | |
| ATGGGGTTTTGGATCTTAGCAATTCTCACAATTCTCATGTATTCCACAGCAGCAAAGTTTAGTAAA | Seq. ID No. 121 |
| CAATCATGGGGCCTGGAAAATGAGGCTTTAATTGTAAGATGTCCTAGACAAGGAAAACCTAGTTA | |
| CACCGTGGATTGGTATTACTCACAAACAAACAAAAGTATTCCCACTCAGGAAAGAAATCGTGTGTT | |
| TGCCTCAGGCCAACTTCTGAAGTTTCTACCAGCTGAAGTTGCTGATTCTGGTATTTATACCTGTAT | |
| TGTCAGAAGTCCCACATTCAATAGGACTGGATATGCGAATGTCACCATATATAAAAAACAATCAGA | |
| TTGCAATGTTCCAGATTATTTGATGTATTCAACAGTATCTGGATCAGAAAAAAATTCCAAAATTTAT | |
| TGTCCTACCATTGACCTCTACAACTGGACAGCACCTCTTGAGTGGTTTAAGAATTGTCAGGCTCTT | |
| CAAGGATCAAGGTACAGGGCGCACAAGTCATTTTTGGTCATTGATAATGTGATGACTGAGGACGC | |
| AGGTGATTACACCTGTAAATTTATACACAATGAAAATGGAGCCAATTATAGTGTGACGGCGACCA | |
| GGTCCTTCACGGTCAAGGATGAGCAAGGCTTTTCTCTGTTTCCAGTAATCGGAGCCCCTGCACAA | |
| AATGAAATAAAGGAAGTGGAAATTGGAAAAAACGCAAACCTAACTTGCTCTGCTTGTTTTGGAAAA | |
| GGCACTCAGTTCTTGGCTGCCGTCCTGTGGCAGCTTAATGGAACAAAAATTACAGACTTTGGTGA | |
| ACCAAGAATTCAACAAGAGGAAGGGCAAAATCAAAGTTTCAGCAATGGGCTGGCTTGTCTAGACA | |
| TGGTTTTAAGAATAGCTGACGTGAAGGAAGAGGATTTATTGCTGCAGTACGACTGTCTGGCCCTG | |
| AATTTGCATGGCTTGAGAAGGCACACCGTAAGACTAAGTAGGAAAAATCCAATTGATCATCATAG | |
| CATCTACTGCATAATTGCAGTATGTAGTGTATTTTTAATGCTAATCAATGTCCTGGTTATCATCCTA | |
| AAAATGTTCTGGATTGAGGCCACTCTGCTCTGGAGAGACATAGCTAAACCTTACAAGACTAGGAA | |
| TGATGGAAAGCTCTATGATGCTTATGTTGTCTACCCACGGAACTACAAATCCAGTACAGATGGGG | |
| CCAGTCGTGTAGAGCACTTTGTTCACCAGATTCTGCCTGATGTTCTTGAAAATAAATGTGGCTATA | |
| CCTTATGCATTTATGGGAGAGATATGCTACCTGGAGAAGATGTAGTCACTGCAGTGGAAACCAAC | |
| ATACGAAAGAGCAGGCGGCACATTTTCATCCTGACCCCTCAGATCACTCACAATAAGGAGTTTGC | |
| CTACGAGCAGGAGGTTGCCCTGCACTGTGCCCTCATCCAGAACGACGCCAAGGTGATACTTATT | |
| GAGATGGAGGCTCTGAGCGAGCTGGACATGCTGCAGGCTGAGGCGCTTCAGGACTCCCTC | |
| CAGCATCTTATGAAAGTACAGGGGACCATCAAGTGGAGGGAGGACCACATTGCCAATAAAAGGT | |
| CCCTGAATTCCAAATTCTGGAAGCACGTGAGGTACCAAATGCCTGTGCCAAGCAAAATTCCCAGA | |
| AAGGCCTCTAGTTTGACTCCCTTGGCTGCCCAGAAGCAATAG | |
| NM_004613: Transglutaminase 2 (TGM2) | |
| AACAGGCGTGACGCCAGTTCTAAACTTGAAACAAAACAAAACTTCAAAGTACACCAAAATAGAACCTCCT | Seq ID No 122 |
| TAAAGCATAAATCTCACGGAGGGTCTCGGCCGCCAGTGGAAGGAGCCACCGCCCCCGCCCCGACCATGGC | |
| CGAGGAGCTGGTCTTAGAGAGGTGTGATCTGGAGCTGGAGACCAATGGCCGAGACCACCACACGGCCGAC | |
| CTGTGCCGGGAGAAGCTGGTGGTGCGACGGGGCCAGCCCTTCTGGCTGACCCTGCACTTTGAGGGCCGCA | |
| ACTACCAGGCCAGTGTAGACAGTCTCACCTTCAGTGTCGTGACCGGCCCAGCCCCTAGCCAGGAGGCCGG | |
| GACCAAGGCCCGTTTTCCACTAAGAGATGCTGTGGAGGAGGGTGACTGGACAGCCACCGTGGTGGACCAG | |
| CAAGACTGCACCCTCTCGCTGCAGCTCACCACCCCGGCCAACGCCCCCATCGGCCTGTATCGCCTCAGCC | |
| TGGAGGCCTCCACTGGCTACCAGGGATCCAGCTTTGTGCTGGGCCACTTCATTTTGCTCTTCAACGCCTG | |
| GTGCCCAGCGGATGCTGTGTACCTGGACTCGGAAGAGGAGCGGCAGGAGTATGTCCTCACCCAGCAGGGC | |
| TTTATCTACCAGGGCTCGGCCAAGTTCATCAAGAACATACCTTGGAATTTTGGGCAGTTTCAAGATGGGA | |
| TCCTAGACATCTGCCTGATCCTTCTAGATGTCAACCCCAAGTTCCTGAAGAACGCCGGCCGTGACTGCTC | |
| CCGGCGCAGCAGCCCCGTCTACGTGGGCCGGGTGGGTAGTGGCATGGTCAACTGCAACGATGACCAGGGT | |
| GTGCTGCTGGGACGCTGGGACAACAACTACGGGGACGGCGTCAGCCCCATGTCCTGGATCGGCAGCGTGG | |
| ACATCCTGCGGCGCTGGAAGAACCACGGCTGCCAGCGCGTCAAGTATGGCCAGTGCTGGGTCTTCGCCGC | |
| CGTGGCCTGCACAGTGCTGAGGTGCCTAGGCATCCCTACCCGCGTCGTGACCAACTACAACTCGGCCCAT | |
| GACCAGAACAGCAACCTTCTCATCGAGTACTTCCGCAATGAGTTTGGGGAGATCCAGGGTGACAAGAGCG | |
| AGATGATCTGGAACTTCCACTGCTGGGTGGAGTCGTGGATGACCAGGCCGGACCTGCAGCCGGGGTACGA | |
| GGGCTGGCAGGCCCTGGACCCAACGCCCCAGGAGAAGAGCGAAGGAACGTACTGCTGTGGCCCAGTTCCA | |
| GTTCGTGCCATCAAGGAGGGCGACCTGAGCACCAAGTACGATGCGCCCTTTGTCTTTGCGGAGGTCAATG | |
| CCGACGTGGTAGACTGGATCCAGCAGGACGATGGGTCTGTGCACAAATCCATCAACCGTTCCCTGATCGT | |
| TGGGCTGAAGATCAGCACTAAGAGCGTGGGCCGAGACGAGCGGGAGGATATCACCCACACCTACAAATAC | |
| CCAGAGGGGTCCTCAGAGGAGAGGGAGGCCTTCACAAGGGCGAACCACCTGAACAAACTGGCCGAGAAGG | |
| AGGAGACAGGGATGGCCATGCGGATCCGTGTGGGCCAGACCATGAACATGGGCAGTGACTTTGACGTCTT | |
| TGCCCACATCACCAACAACACCGCTGAGGAGTACGTCTGCCGCCTCCTGCTCTGTGCCCGCACCGTCAGC | |
| TACAATGGGATCTTGGGGCCCGAGTGTGGCACCAAGTACCTGCTCAACCTAACCCTGGAGCCTTTCTCTG | |
| AGAAGAGCGTTCCTCTTTGCATCCTCTATGAGAAATACCGTGACTGCCTTACGGAGTCCAACCTCATCAA | |
| GGTGCGGGCCCTCCTCGTGGAGCCAGTTATCAACAGCTACCTGCTGGCTGAGAGGGACCTCTACCTGGAG | |
| AATCCAGAAATCAAGATCCGGATCCTTGGGGAGCCCAAGCAGAAACGCAAGCTGGTGGCTGAGGTGTCCC | |
| TGCAGAACCCGCTCCCTGTGGCCCTGGAAGGCTGCACCTTCACTGTGGAGGGGGCCGGCCTGACTGAGGA | |
| GCAGAAGACGGTGGAGATCCCAGACCCCGTGGAGGCAGGGGAGGAAGTTAAGGTGAGAATGGACCTCGTG | |
| CCGCTCCACATGGGCCTCCACAAGCTGGTGGTGAACTTCGAGAGCGACAAGCTGAAGGCTGTGAAGGGCT | |
| TCCGGAATGTCATCATTGGCCCCGCCTAAGGGACCCCTGCTCCCAGCCTGCTGAGAGCCCCCACCTTGAT | |
| CCCAATCCTTATCCCAAGCTAGTGAGCAAAATATGCCCCTTATTGGGCCCCAGACCCCAGGGCAGGGTGG | |
| GCAGCCTATGGGGGCTCTCGGAAATGGAATGTGCCCCTGGCCCATCTCAGCCTCCTGAGCCTGTGGGTCC | |
| CCACTCACCCCCTTTGCTGTGAGGAATGCTCTGTGCCAGAAACAGTGGGAGCCCTGACCTGTGCTGACTG | |
| GGGCTGGGGTGAGAGAGGAAAGACCTACATTCCCTCTCCTGCCCAGATGCCCTTTGGAAAGCCATTGACC | |
| ACCCACCATATTGTTTGATCTACTTCATAGCTCCTTGGAGCAGGCAAAAAAGGGACAGCATGCCCTTGGC | |
| TGGATCAGGAATCCAGCTCCCTAGACTGCATCCCGTACCTCTTCCCATGACTGCACCCAGCTCCAGGGGC | |
| CCTTGGGACACCCAGAGCTGGGTGGGGACAGTGATAGGCCCAAGGTCCCCTCCACATCCCAGCAGCCCAA | |
| GCTTAATAGCCCTCCCCCTCAACCTCACCATTGTGAAGCACCTACTATGTGCTGGGTGCCTCCCACACTT | |
| GCTGGGGCTCACGGGGCCTCCAACCCATTTAATCACCATGGGAAACTGTTGTGGGCGCTGCTTCCAGGAT | |
| AAGGAGACTGAGGCTTAGAGAGAGGAGGCAGCCCCCTCCACACCAGTGGCCTCGTGGTTATAAGCAAGGC | |
| TGGGTAATGTGAAGGCCCAAGAGCAGAGTCTGGGCCTCTGACTCTGAGTCCACTGCTCCATTTATAACCC | |
| CAGCCTGACCTGAGACTGTCGCAGAGGCTGTCTGGGGCCTTTATCAAAAAAAGACTCAGCCAAGACAAGG | |
| AGGTAGAGAGGGGACTGGGGGACTGGGAGTCAGAGCCCTGGCTGGGTTCAGGTCCCACGTCTGGCCAGCG | |
| ACTGCCTTCTCCTCTCTGGGCCTTTGTTTCCTTGTTGGTCAGAGGAGTGATTGAACCTGCTCATCTCCAA | |
| GGATCCTCTCCACTCCATGTTTGCAATACACAATTCC | |
| NM_012323: V-maf musculo aponeurotic fibrosarcoma oncogene homolog F (MAFF) | |
| AGTAATTCCGGGAAGCTCGCCTTACAACTCCGCGCGGCCTCGGCCCCCTGCGCCGCCCGCCCC | Seq. ID No. 123 |
| ACAACAAAACTCAGCGCAGCGCTCCCGGGCGCCCGGTTCAGAGCGACCTGCGGCTCAGAGCGG | |
| AGGGGAGACTGACCGGAGCGCGGATCGGGACAGCGGCCGGGACAGCGGCGAGACGCGCGTGT | |
| GTGAGCGCGCCGGACCAAGCGGGCCCAGAAGCGGGTCTGCAGCCCAGAGGGC | |
| ACCTTCTGCAAACATGTCTGTGGATCCCCTATCCAGCAAAGCTCTAAAGATCAAGCGAGAGCTGA | |
| GCGAGAACACGCCGCACCTGTCGGACGAGGCGCTGATGGGGCTGTCGGTGCGCGAGCTGAAC | |
| CGGCATCTGCGCGGGCTCTCCGCCGAGGAGGTGACACGGCTCAAGCAGCGGCGCCGCACACT | |
| CAAAAACCGTGGCTACGCCGCCAGCTGCCGCGTGAAGCGCGTGTGCCAGAAGGAGGAGCTGCA | |
| GAAGCAGAAGTCGGAGCTGGAGCGCGAGGTGGACAAGCTGGCGCGCGAGAACGCCGCCATGC | |
| GCCTGGAGCTCGACGCGCTGCGCGGCAAGTGCGAGGCGCTGCAGGGCTTCGCGCGCTCCGTG | |
| GCCGCCGCCCGCGGGCCCGCCACGCTCGTGGCGCCGGCCAGCGTCATCACCATCGTCAAGTC | |
| CACCCCGGGCTCGGGGTCTGGCCCCGCCCACGGCCCGGACCCCGCCCACGGCCCGGCCTCCT | |
| GCTCCTAGTGCCCGCCCCCGCCATGCCTCAGCCACGCCCCTCCGGCCTCAGCTCCCTCCCCAA | |
| AGTGCCTGAGCGCCGCCTCTGTGCCCAGGTCCCATTTCTCTGCAGCACTGGCCCCTTGGTGCAC | |
| ACACATTCCCTTCGTGGGCCCTGTCTTCCTCTTGCAGCCCCCCAAACTGGGACCGAATGACCCT | |
| GGGAAGGGGAACTTGGGTAGGTTGGGGATGGGGCAGAGGTCTGGATCTGGGATCGCCCTTGGC | |
| TGAAAGTTTAGCCTTTTTAGATTGAGAGATACAGAGCCGGCTTAGAGAACAGCTGTTGGGGGAGA | |
| AGAGGGCACCCCTCATCTTGGAAACTGCTCTTATTGTGCCAATATGCCCTCCAAACCCTCCCAGG | |
| ATTCAAAGCTAGGTTTGGCTGTCTGTGACTTACGGGACCGTCCTGCTGAGAAATTGCACTGAAGA | |
| GATGCCCCCACCTCTGGTTGGGCCTGGGGGTGCCTGGCCTTCCGAAACTAAAAGAGTGGGTGG | |
| GAAGACTAGTGAAACCCAGTTCACGGATGGGGAAACAGGCCTGAGGTCACATTTCACTTAGTGG | |
| TTGTGTTGGGACCAAAACCTGGGTGTCCTCACTGCTGCCCTGAGTCCAGCCATGGTTTTCAGGG | |
| GGACAGTGGACAGGGACTCAGAAATGTGGTGGGAGGGCCTCCCTGGCTTGGGAGACCGCTCTC | |
| TGCAAGGGAGGGGGAGAGAAGCAGAGGGAGAGAGAAGGTGACACGGATGGAAGAGTGGGAAG | |
| GAGCTGGCCTGGCTCAGCCCTAGGCTGTCCCTGCAGCCAGGGTGTCCGGGGGCTGGCCAGTCA | |
| GAGAAAGGGGGCCATGGACTGCTGTGGCAAATAGGGAGACAAGGAGACAGACCCTGCAGTCCT | |
| ACTACAGTCTGGAGTGGGGTCCTAAGAAGAAGGGTCCCACCTCAACCCCTGTCAGTGTCCACTG | |
| TGGGGTGGGGGCTGACCCCTGCCTTTGATTGTCATTCTCCTGGGAAGCCCAGTCTCAGTCCCTC | |
| CCCCAACACTGTCCACACTGCCCCTCCCCACTGTTTATTTATTGCACGGATCTAAGTTATTCTCCC | |
| CAGCCAGAGCCCGAGCTCCTGCTCCCTGGGAAAAGTGGCGTATGGCCCTGAGCTGGGCTTTATA | |
| TTTTATATCTGCAAATAAATCACATTTTATCTTATATTTAGGGAAAGCCGGAGAGCAACAACAAAAA | |
| ATGTTTAAGCCGGGCGCGGTGGCTCACATCTGTAATCCCAGCACTTTGGGAGTCCAAGGAGGGG | |
| GATCGCTTGAGTCCAGGAGTTTGAGACCAGCCTGGACAACATGGTGAAACCCCGTCTCTACAAAA | |
| AATACAAAAATTAGCCATGCATGGTGGCTCATGCCTGTAGTCCCAGCTACTTGGGAGGCTGAGGC | |
| AGGAGGATCACTTAAGCCCAGAAGGCAGAGGTTGTAGTGAGCTGAGATCGCACCACTGCACTCC | |
| AGCCTGGGCAACATAGCAAAATCCTGTCTCAAAAAAAAAGTTAAAAAATATTGCCCGGCTCCTAGA | |
| ATTTATTTATTTCCTGACTTACAGCAAGCGAGTTATCGTCTTCTGTATTTTGTAGACTTTCTAAATAA | |
| AGTCAAATTCTTTCTTTTTCCACAGAGAAAAAAA | |

NM_001085: Serine or cysteine proteinase inhibitor clade A member 3 (SERPINA3)

| | |
|---|---|
| GGAATTCCCTGGAGCAGAGTTGAGAATGGAGAGAATGTTACCTCTCCTGGCTCTGGGGCTCTTG | Seq. ID No. 124 |
| GCGGCTGGGTTCTGCCCTGCTGTCCTCTGCCACCCTAACAGCCCACTTGACGAGGAGAATCTGA | |
| CCCAGGAGAACCAAGACCGAGGGACACACGTGGACCTCGGATTAGCCTCCGCCAACGTGGACT | |
| TCGCTTTCAGCCTGTACAAGCAGTTAGTCCTGAAGGCCCTTGATAAGAATGTCATCTTCTCCCCA | |
| CTGAGCATCTCCACCGCCTTGGCCTTCCTGTCTCTGGGGGCCCATAATACCACCCTGACAGAGA | |
| TTCTCAAGGCCTCGAGTTCACCTCACGGAGACTTACTGAGGCAGAAATTCACTCAGAGCTTCCAG | |
| CACCTCCGCGCACCCTCAATCAGTTCCAGCGATGAGCTGCAGCTGAGTATGGGAAATGCCATGT | |
| TTGTCAAAGAGCAACTCAGTCTGCTGGACAGGTTCACGGAGGATGCCAAGAGGCTGTATGGCTC | |
| CGAGGCCTTTGCCACTGACTTTCAGGACTCAGCTGCAGCTAAGAAGCTCATCAACGACTACGTGA | |
| AGAATGGAACTAGGGGGAAAATCACAGATCTGATCAAGGACCCCGACTCGCAGACAATGATGGT | |
| CCTGGTGAATTACATCTTCTTTAAAGCCAAATGGGAGATGCCCTTTGACCCCCAAGATACTCATCA | |
| GTCAAGGTTCTACTTGAGCAAGAAAAAGTGGGTAATGGTGCCCATGATGAGTTTGCATCACCTGA | |
| CTATACCTTACTTCCGGGACGAGGAGCTGTCCTGCACCGTGGTGGAGCTGAAGTACACAGGCAA | |
| TGCCAGCGCACTCTTCATCCTCCCTGATCAAGACAAGATGGAGGAAGTGGAAGCCATGCTGCTC | |
| CCAGAGACCCTGAAGCGGTGGAGAGACTCTCTGGAGTTCAGAGAGATAGGTGAGCTCTACCTGC | |
| CAAAGTTTTCCATCTCGAGGGACTATAACCTGAACGACATACTTCTCCAGCTGGGCATTGAGGAA | |
| GCCTTCACCAGCAAGGCTGACCTGTCAGGGATCACAGGGGCCAGGAACCTAGCAGTCTCCCAG | |
| GTGGTCCATAAGGTCGTGTCTGATGTATTTGAGGAGGGCACAGAAGCATCTGCTGCCACAGCAG | |
| TCAAAATCACCCTCCTTTCTGCATTAGTGGAGACAAGGACCATTGTGCGTTTCAACAGGCCCTTC | |
| CTGATGATCATTGTCCCTACAGACAGCCAGAACATCTTCTTCATGAGCAAAGTCACCAATCCCAG | |
| CAAGCCTAGAGCTTGCATCAAGCAGTGGGGCTCTCAGTAAGGAACTTGGAATGCAAGCTGGATG | |
| CCTGGGTCTCTGGGCACAGCTGGCCCCTGTGCACCGTAGTGGCCATGGCATGTGTGGCCCTGT | |
| CTGCTTATCCTTGGAAGGTGACAGCGATTCCCTGTGAAGCTCTCACACGCACAGGGGCCCATGG | |
| ACTCTTCAGTCTGGAGGGTCCTGGCCTCCTGACAGCAATAAATAATTTCGTTGGCC | |
| NM_001511: GRO1 oncogene melanoma growth stimulating activity alpha (GRO1) | |
| CACAGAGCCCGGGCCGCAGGCACCTCCTCGCCAGCTCTTCCGCTCCTCTCACAGCCGCCAGAC | Seq. ID No. 125 |
| CCGCCTGCTGAGCCCCATGGCCCGCGCTGCTCTCTCCGCCGCCCCCAGCAATCCCCGGCTCCT | |
| GCGAGTGGCACTGCTGCTCCTGCTCCTGGTAGCCGCTGGCCGGCGCGCAGCAGGAGCGTCCGT | |
| GGCCACTGAACTGCGCTGCCAGTGCTTGCAGACCCTGCAGGGAATTCACCCCAAGAACATCCAA | |
| AGTGTGAACGTGAAGTCCCCCGGACCCCACTGCGCCCAAACCGAAGTCATAGCCACACTCAAGA | |
| ATGGGCGGAAAGCTTGCCTCAATCCTGCATCCCCCATAGTTAAGAAAATCATCGAAAAGATGCTG | |
| AACAGTGACAAATCCAACTGACCAGAAGGGAGGAGGAAGCTCACTGGTGGCTGTTCCTGAAGGA | |
| GGCCCTGCCCTTATAGGAACAGAAGAGGAAAGAGAGACACAGCTGCAGAGGCCACCTGGATTGT | |
| GCCTAATGTGTTTGAGCATCGCTTAGGAGAAGTCTTCTATTTATTTATTTATTCATTAGTTTTGAAG | |
| ATTCTATGTTAATATTTTAGGTGTAAAATAATTAAGGGTATGATTAACTCTACCTGCACACTGTCCT | |
| ATTATATTCATTCTTTTTGAAATGTCAACCCCAAGTTAGTTCAATCTGGATTCATATTTAATTTGAAG | |
| GTAGAATGTTTTCAAATGTTCTCCAGTCATTATGTTAATATTTCTGAGGAGCCTGCAACATGCCAG | |
| CCACTGTGATAGAGGCTGGCGGATCCAAGCAAATGGCCAATGAGATCATTGTGAAGGCAGGGGA | |
| ATGTATGTGCACATCTGTTTTGTAACTGTTTAGATGAATGTCAGTTGTTATTTATTGAAATGATTTC | |
| ACAGTGTGTGGTCAACATTTCTCATGTTGAAACTTTAAGAACTAAAATGTTCTAAATATCCCTTGGA | |
| CATTTTATGTCTTTCTTGTAAGGCATACTGCCTTGTTTAATGGTAGTTTTACAGTGTTTCTGGCTTA | |
| GAACAAAGGGGCTTAATTATTGATGTTTTCATAGAGAATATAAAAATAAAGCACTTATAG | |
| NM_000591: CD14 antigen (CD14) | |
| CCGGCCGGCCGAAGAGTTCACAAGTGTGAAGCCTGAAGCCGCCGGGTGCCGCTGTGTAGAAAG | Seq. ID No. 126 |
| AAGCTAAAGCACTTCCAGAGCCTGCTGAGCTCAGAGGTTCGGAAGACTTATCGACCATGGAGCG | |
| CGCGTCCTGCTTGTTGCTGCTGCTGCTGCCGCTGGTGCACGTCTCTGCGACCACGCCAGAACCT | |
| TGTGAGCTGGACGATGAAGATTTCCGCTGCGTCTGCAACTTCTCCGAACCTCAGCCCGACTGGT | |
| CCGAAGCCTTCCAGTGTGTGTCTGCAGTAGAGGTGGAGATCCATGCCGGCGGTCTCAACCTAGA | |
| GCCGTTTCTAAAGCGCGTCGATGCGGACGCCGACCCGCGGCAGTATGCTGACACGGTCAAGGC | |
| TCTCCGCGTGCGGCGGCTCACAGTGGGAGCCGCACAGGTTCCTGCTCAGCTACTGGTAGGCGC | |
| CCTGCGTGTGCTAGCGTACTCCCGCCTCAAGGAACTGACGCTCGAGGACCTAAAGATAACCGGC | |
| ACCATGCCTCCGCTGCCTCTGGAAGCCACAGGACTTGCACTTTCCAGCTTGCGCCTACGCAACG | |
| TGTCGTGGGCGACAGGGCGTTCTTGGCTCGCCGAGCTGCAGCAGTGGCTCAAGCCAGGCCTCA | |
| AGGTACTGAGCATTGCCCAAGCACACTCGCCTGCCTTTTCCTGCGAACAGGTTCGCGCCTTCCC | |
| GGCCCTTACCAGCCTAGACCTGTCTGACAATCCTGGACTGGGCGAACGCGGACTGATGGCGGCT | |
| CTCTGTCCCCACAAGTTCCCGGCCATCCAGAATCTAGCGCTGCGCAACACAGGAATGGAGACGC | |
| CCACAGGCGTGTGCGCCGCACTGGCGGCGGCAGGTGTGCAGCCCCACAGCCTAGACCTCAGCC | |
| ACAACTCGCTGCGCGCCACCGTAAACCCTAGCGCTCCGAGATGCATGTGGTCCAGCGCCCTGAA | |
| CTCCCTCAATCTGTCGTTCGCTGGGCTGGAACAGGTGCCTAAAGGACTGCCAGCCAAGCTCAGA | |
| GTGCTCGATCTCAGCTGCAACAGACTGAACAGGGCGCCGCAGCCTGACGAGCTGCCCGAGGTG | |
| GATAACCTGACACTGGACGGGAATCCCTTCCTGGTCCCTGGAACTGCCCTCCCCCACGAGGGCT | |
| CAATGAACTCCGGCGTGGTCCCAGCCTGTGCACGTTCGACCCTGTCGGTGGGGGTGTCGGGAA | |
| CCCTGGTGCTGCTCCAAGGGGCCCGGGGCTTTGCCTAAGATCCAAGACAGAATAATGAATGGAC | |
| TCAAACTGCCTTGGCTTCAGGGGAGTCCCGTCAGGACGTTGAGGACTTTTCGACCAATTCAACCC | |
| TTTGCCCCACCTTTATTAAAATCTTAAAC | |
| NM_015319: Tensin 2 (KIAA1075) | |
| GCACATTCTTTCAAGTGACAGCTATAGCCTGTCCCAGGGGCTGCTGTCCACAGCTTGGGGCTGA | Seq. ID No. 127 |
| AGACTCCCAGGCCATTAACCCCTTAGCTTTTAGGAAGATTACTCCCCCTTTTTCAAGGCCCCATCC | |
| ACCTCCCTCCCTTGACTCCCAGGACGGGAAGTTGGCCATGTTCCCAGGAGGGAGGCCGGAGGC | |
| CCATGGATGGGGGTGGAGTATGTGTTGGGAGGGGGGACCTCCTGTCCAGTCCTCAGGCCCTGG | |
| GACAGCTGCTGAGGAAGGAGAGCAGACCCAGGAGAGCCATGAAGCCTAGGAAAGCTGAGCCTC | |
| ATAGCTTCCGGGAGAAGGTTTTCCGGAAGAAACCTCCAGTCTGTGCAGTATGTAAGGTGACCATC | |
| GATGGGACAGGCGTTTCGTGCAGAGTCTGCAAGGTGGCGACGCACAGAAAATGTGAAGCAAAG | |
| GTGACTTCAGCCTGTCAGGCCTTGCCTCCCGTGGAGTTGCGGCGAAACACGGCCCCAGTCAGG | |
| CGCATAGAGCACCTGGGATCCACCAAATCTCTGAACCACTCAAAGCAGCGCAGCACTCTGCCCA | |
| GGAGCTTCAGCCTGGACCCGCTCATGGAGCGGCGCTGGGACTTAGACCTCACCTACGTGACGG | |
| AGCGCATCTTGGCCGCCGCCTTCCCCGCGCGGCCCGATGAACAGCGGCACCGGGGCCACCTG | |
| CGCGAGCTGGCCCATGTGCTGCAATCCAAGCACCGGGACAAGTACCTGCTCTTCAACCTTTCAG | |
| AGAAAAGGCATGACCTGACCCGCTTAAACCCCAAGGTTCAAGACTTCGGCTGGCCTGAGCTGCA | |
| TGCTCCACCCCTGGACAAGCTGTGCTCCATCTGCAAAGCCATGGAGACATGGCTCAGTGCTGAC | |
| CCACAGCACGTGGTCGTACTATACTGCAAGGGAAACAAGGGCAAGCTTGGGGTCATCGTTTCTG | |
| CCTACATGCACTACAGCAAGATCTCTGCAGGGGCGGACCAGGCACTGGCCACTCTTACCATGCG | |
| GAAATTCTGCGAGGACAAGGTGGCCACAGAACTGCAGCCCTCCCAGCGTCGATATATCAGCTAC | |
| TTCAGTGGGCTGCTATCTGGCTCCATCAGAATGAACAGCAGCCCTCTCTTCCTGCACTATGTGCT | |
| CATCCCCATGCTGCCAGCCTTTGAACCTGGCACAGGCTTCCAGCCCTTCCTTAAAATCTACCAGT | |
| CCATGCAGCTTGTCTACACATCTGGAGTCTATCACATTGCAGGCCCTGGTCCCCAGCAGCTTTGC | |
| ATCAGCCTGGAGCCAGCCCTCCTCCTCAAAGGCGATGTCATGGTAACATGTTATCACAAGGGTG | |
| GCCGGGGCACAGACCGGACCCTCGTGTTCCGAGTCCAGTTCCACACCTGCACCATCCACGGAC | |
| CACAGCTCACTTTCCCCAAGGACCAGCTTGACGAGGCCTGGACTGATGAGAGGTTCCCCTTCCA | |
| AGCCTCCGTGGAGTTTGTCTTCTCCTCCAGCCCCGAGAAGATCAAAGGCAGCACTCCACGGAAC | |
| GACCCCTCGGTCTCTGTCGACTACAACACCACTGAGCCAGCCGTGCGCTGGGACTCCTATGAGA | |
| ACTTCAACCAGCACCACGAGGACAGTGTGGATGGCTCCTTGACCCACACCCGGGGTCCCCTGGA | |
| TGGCAGTCCTTATGCCCAGGTGCAGCGGCCTCCCCGGCAGACCCCCCCGGCACCCTCTCCAGA | |
| GCCTCCACCACCCCCCATGCTCTCTGTCAGCAGCGACTCAGGCCATTCCTCCACGCTGACCACA | |
| GAGCCGGCTGCTGAGTCCCCTGGCCGGCCGCCCCCTACAGCTGCTGAACGGCAGGAGCTGGAT | |
| CGCCTCCTAGGAGGCTGCGGAGTGGCCAGTGGGGGCCGGGGAGCTGGGCGCGAGACGGCCAT | |
| CCTAGATGACGAAGAGCAGCCCACTGTGGGCGGAGGCCCCCACCTCGGAGTGTATCCAGGCCA | |
| TAGGCCTGGCCTCAGCCGCCACTGCTCCTGCCGCCAGGGCTACCGGGAGCCCTGCGGGGTTCC | |
| CAATGGGGGCTACTACCGGCCAGAGGGAACCCTGGAGAGGAGGCGACTGGCCTACGGGGGCT | |
| ATGAGGGATCCCCCCAGGGCTACGCCGAGGCCTCGATGGAGAAGAGGCGCCTCTGGCGATCGC | |
| TGTCAGAGGGGCTATACCCCTACCCACCTGAGATGGGGAAACCAGCCACTGGGGACTTTGGCTA | |
| CCGCGCCCCAGGCTACCGGGAGGTGGTCATCCTGGAGGACCCTGGGCTGCCTGCCCTATACCC | |
| ATGCCCAGCCTGCGAGGAGAAGCTGGCGCTGCCTACAGCAGCCTTGTATGGACTGCGGCTGGA | |
| GAGGGAGGCTGGAGAAGGGTGGGCAAGTGAGGCTGGCAAGCCTCTCCTGCACCCAGTGCGGC | |
| CTGGGCACCCGCTGCCTCTGCTCTTGCCTGCCTGTGGGCATCACCATGCCCCGATGCCTGACTA | |
| CAGCTGCCTGAAGCCACCCAAGGCAGGCGAGGAAGGGCACGAGGGCTGCTCCTACACCATGTG | |
| CCCCGAAGGCAGGTATGGGCATCCAGGGTACCCTGCCCTGGTGACATACAGCTATGGAGGAGC | |
| AGTTCCCAGTTACTGCCCAGCATATGGCCGTGTGCCTCATAGCTGTGGCTCTCCAGGAGAGGGC | |
| AGAGGGTATCCCAGCCCTGGTGCCCACTCCCCACGGGCTGGCTCCATTTCCCCGGGCAGCCCG | |
| CCCTATCCACAATCTAGGAAGCTGAGCTACGAGATCCCTACGGAGGAGGGAGGGGACAGGTACC | |
| CATTGCCTGGGCACCTGGCCTCAGCAGGACCTTTGGCATCTGCAGAGTCGCTGGAGCCGGTGTC | |
| CTGGAGGGAGGGCCCCAGTGGGCACAGCACACTGCCTCGGTCTCCCCGAGATGCCCCATGCAG | |
| TGCTTCGTCAGAGTTGTCTGGTCCCTCCACGCCCCTGCACACCAGCAGTCCAGTCGAGGGCAAG | |
| GAAAGCACCCGGCGACAGGACACCAGGTCCCCCACCTCAGCGCCCACTCAGAGACTGAGTCCT | |
| GGCGAGGCCTTGCCCCCTGTTTCCCAGGCAGGCACCGGAAAGGCCCCTGAGCTGCCGTCGGGA | |
| AGTGGGCCTGAGCCTCTGGCCCCTAGCCCAGTCTCTCCGACCTTCCCTCCCAGCTCGCCCAGTG | |
| ACTGGCCTCAGGAAAGGAGTCCAGGGGGCCACTCAGATGGCGCCAGTCCTCGGAGCCCTGTGC | |
| CCACCACACTTCCTGGCCTCCGCCACGCCCCCTGGCAAGGCCCTCGAGGCCCCCCCGACAGCC | |
| CAGATGGGTCTCCCCTCACTCCTGTGCCTTCCCAGATGCCCTGGCTTGTGGCCAGCCCAGAGCC | |
| GCCTCAGAGCTCACCTACACCTGCTTTCCCCCTGGCTGCCTCCTATGACACCAATGGCCTTAGCC | |
| AGCCCCCACTTCCTGAGAAACGCCACCTGCCCGGGCCGGGGCAACAGCCAGGACCCTGGGGCC | |
| CAGAGCAGGCATCATCGCCAGCCAGAGGCATCAGTCACCATGTCACCTTCGCACCTCTGCTCTC | |
| AGATAATGTCCCCCAAACCCCAGAGCCTCCTACACAAGAGAGCCAAAGCAATGTCAAGTTTGTCC | |
| AGGATACATCCAAGTTCTGGTACAAGCCACACCTGTCCCGTGACCAAGCCATTGCCCTGCTGAAG | |
| GACAAGGACCCTGGGGCCTTCCTGATCAGGGACAGTCATTCATTCCAAGGAGCTTATGGGCTGG | |
| CCCTCAAGGTGGCCACACCGCCACCCAGTGCCCAGCCCTGGAAAGGGGACCCCGTGGAACAGC | |
| TGGTCCGCCATTTCCTCATCGAGACTGGGCCCAAAGGGGTGAAGATCAAGGGCTGCCCCAGTGA | |
| GCCCTACTTTGGCAGCCTGTCCGCCTTGGTCTCCCAGCACTCCATCTCCCCCATCTCCCTGCCCT | |
| GCTGCCTGCGCATTCTCAGCAAAGATCCTCTGGAAGAGACCCCAGAGGCTCCAGTGCCCACCAA | |
| CATGAGCACAGCGGCAGACCTCCTGCGTCAGGGTGCTGCCTGCAGCGTGCTCTACTTGACCTCA | |
| GTGGAGACAGAGTCACTGACGGGCCCCCAAGCTGTGGCCCGGGCCAGCTCTGCAGCTCTGAGC | |
| TGTAGCCCCCGCCCGACACCAGCTGTTGTCCACTTCAAGGTGTCAGCCCAGGGCATTACACTGA | |
| CGGACAACCAAAGGAAGCTCTTCTTTCGCCGCCATTATCCAGTGAACAGCATCACCTTCTCCAGC | |
| ACTGACCCTCAAGACCGGAGATGGACCAACCCAGACGGGACCACCTCCAAGATCTTTGGTTTCG | |
| TGGCCAAGAAGCCGGGAAGCCCCTGGGAGAATGTGTGTCACCTCTTTGCAGAGCTTGACCCAGA | |
| TCAGCCTGCTGGCGCCATTGTCACCTTCATCACCAAAGTTCTACTGGGCCAGAGAAAATGAAGGA | |
| AGGCCACAAGCTCAGAGCCCACATCAACACTGCCCCCCTCCCAGCACCCCACAGCCCTCACATC | |
| CCCTGGCCTGGACCCAGGAGACCCAGGAGAAAGCACCCTCCCTTAGGAATGAGGAGTGGGCAT | |
| CAGGCCTGGGACACTGCTCTCCTTCCCCGCCCCCAGCCTGCTAAGTTAAGTGGACAGGCCCACA | |
| AGATGACCTTGCATGTGAGCAGATGGCAGAGATGGGTGTGTGAGGGGTGAGGAGGCATCAGCA | |
| GTTGAGCCCCGAAGGAGATCAGGCAGCCCCACCTGCAGGAGAACGTCAGCCCTCCAGGGGATC | |
| AGCCCCTGCCAGTTCCACCCAGCTGCAGGTGCCAGCACGGCAGGGATGGGAGAGGGGTGGGG | |
| AGCGAGTCACTGCCTCCTCTGAGCAGAGATTCAGAGTAGGATCACATGAATAGGGGAAAAAAGA | |
| GAGTCTATTTTTGTCTAATAATAAAGAATTTCTATAAACTTT | |
| NM_001276: Chitinase 3-like 1, cartilage glycoprotein-39 (CHI3L1) | |
| AGTGGAGTGGGACAGGTATATAAAGGAAGTACAGGGCCTGGGGAAGAGGCCCTGTCTAGGTAG | Seq. ID No. 128 |
| CTGGCACCAGGAGCCGTGGGCAAGGGAAGAGGCCACACCCTGCCCTGCTCTGCTGCAGCCAGA | |
| ATGGGTGTGAAGGCGTCTCAAACAGGCTTTGTGGTCCTGGTGCTGCTCCAGTGCTGCTCTGCAT | |
| ACAAACTGGTCTGCTACTACACCAGCTGGTCCCAGTACCGGGAAGGCGATGGGAGCTGCTTCCC | |
| AGATGCCCTTGACCGCTTCCTCTGTACCCACATCATCTACAGCTTTGCCAATATAAGCAACGATCA | |
| CATCGACACCTGGGAGTGGAATGATGTGACGCTCTACGGCATGCTCAACACACTCAAGAACAGG | |
| AACCCCAACCTGAAGACTCTCTTGTCTGTCGGAGGATGGAACTTTGGGTCTCAAAGATTTTCCAA | |
| GATAGCCTCCAACACCCAGAGTCGCCGGACTTTCATCAAGTCAGTACCGCCATTCCTGCGCACC | |
| CATGGCTTTGATGGGCTGGACCTTGCCTGGCTCTACCCTGGACGGAGAGACAAACAGCATTTTA | |
| CCACCCTAATCAAGGAAATGAAGGCCGAATTTATAAAGGAAGCCCAGCCAGGGAAAAAGCAGCT | |
| CCTGCTCAGCGCAGCACTGTCTGCGGGGAAGGTCACCATTGACAGCAGCTATGACATTGCCAAG | |
| ATATCCCAACACCTGGATTTCATTAGCATCATGACCTACGATTTTCATGGAGCCTGGCGTGGGAC | |
| CACAGGCCATCACAGTCCCCTGTTCCGAGGTCAGGAGGATGCAAGTCGTGACAGATTCAGCAAC | |
| ACTGACTATGCTGTGGGGTACATGTTGAGGCTGGGGGCTCCTGCCAGTAAGCTGGTGATGGGCA | |
| TCCCCACCTTCGGGAGGAGCTTCACTCTGGCTTCTTCTGAGACTGGTGTTGGAGCCCCAATCTCA | |
| GGACCGGGAATTCCAGGCCGGTTCACCAAGGAGGCAGGGACCCTTGCCTACTATGAGATCTGTG | |
| ACTTCCTCCGCGGAGCCACAGTCCATAGAACCCTCGGCCAGCAGGTCCCCTATGCCACCAAGGG | |
| CAACCAGTGGGTAGGATACGACGACCAGGAAAGCGTCAAAAGCAAGGTGCAGTACCTGAAGGAT | |
| AGGCAGCTGGCAGGCGCCATGGTATGGGCCCTGGACCTGGATGACTTCCAGGGCTCCTTCTGC | |
| GGCCAGGATCTGCGCTTCCCTCTCACCAATGCCATCAAGGAT | |
| GCACTCGCTGCAACGTAGCCCTCTGTTCTGCACACAGCACGGGGGCCAAGGATGCCCCGTCCC | |
| CCTCTGGCTCCAGCTGGCCGGGAGCCTGATCACCTGCCCTGCTGAGTCCCAGGCTGAGCCTCA | |
| GTCTCCCTCCCTTGGGGCCTATGCAGAGGTCCACAACACACAGATTTGAGCTCAGCCCTGGTGG | |
| GCAGAGAGGTAGGGATGGGGCTGTGGGGATAGTGAGGCATCGCAATGTAAGACTCGGGATTAG | |
| TACACACTTGTTGATGATTAATGGAAATGTTTACAGATCCCCAAGCCTGGCAAGGGAATTTCTTCA | |
| ACTCCCTGCCCCCTAGCCCTCCTTATCAAAGGACACCATTTTGGCAAGCTCTATCACCAAGGAGC | |
| CAAACATCCTACAAGACACAGTGACCATACTAATTATACCCCCTGCAAAGCCAGGTTGAAACCTTC | |
| ACTTAGGAACGTAATCGTGTCCCCTATCCTACTTCCCCTTCCTAATTCCACAGCTGCTCAATAAAG | |
| TACAAGAGTTTAACAGTGTGTTGGCGCTTTGCTTTGGTCTATCTTTGAGCGCCCACTAGACCCACT | |
| GGACTCACCTCCCCCATCTCTTCTGGGTTCCTTCCTCTGAGCCTTGGGACCCCTGAGCTTGCAGA | |
| GATGAAGGCCGCCATGTT | |
| NM_004353: Serine or cysteine proteinase inhibitor clade H (SERPINH1) | |
| GGTCCTCTGTGGTGCACAGCCCACCCCCCAGCCATGCGCTCTCTCCTTCTGGGCACCTTATGCC | Seq. ID No. 129 |
| TCCTGGCTGTGGCCCTGGCAGCCGAGGTGAAGAAACCTGTAGAGGCCGCAGCCCCTGGTACTG | |
| CGGAGAAGCTGAGTTCCAAGGCGACCACACTGGCAGAGCCCAGCACAGGCCTGGCCTTCAGCC | |
| TGTATCAGGCAATGGCCAAGGACCAGGCAGTGGAGAACATCCTGGTGTCACCCGTGGTGGTGG | |
| CCTCGTCGCTGGGTCTCGTGTCGCTGGGCGGCAAGGCGACCACGGCGTCGCAGGCCAAGGCA | |
| GTGCTGAGCGCCGAGCAGCTGCGCGACGAGGAGGTGCACGCCGGCCTGGGTGAGCTGCTGCG | |
| CTCACTCAGCAACTCGACGGCGCGCAACGTGACCTGGAAGCTGGGCAGCCGACTGTACGGACC | |
| CAGCTCAGTGAGCTTCGCTGATGACTTCGTGCGCAGCAGCAAGCAGCACTACAACTGCGAGCAC | |
| TCCAAGATCAACTTCCCGGACAAGCGCAGCGCGCTGCAGTCCATCAACGAGTGGGCCGCGCAG | |
| ACCACCGACGGCAAGCTGCCCGAGGTCACCAAGGACGTGGAGCGCACGGACGGCGCCCTGCTA | |
| GTCAACGCCATGTTCTTCAAGCCACACTGGGATGAGAAATTCCACCACAAGATGGTGGACAACCG | |
| TGGCTTCATGGTGACTCGGTCCTATACTGTGGGTGTTACGATGATGCACCGGACAGGCCTCTACA | |
| ACTACTACGACGACGAGAAGGAGAAGCTGCAGCTGGTGGAGATGCCCCTGGCTCACAAGCTCTC | |
| CAGCCTCATCATCCTCATGCCCCATCACGTGGAGCCTCTCGAGCGCCTTGAAAAGCTGCTAACCA | |
| AAGAGCAGCTGAAGATCTGGATGGGGAAGATGCAGAAGAAGGCTGTTGCCATCTCCTTGCCCAA | |
| GGGTGTGGTGGAGGTGACCCATGACCTGCAGAAACACCTGGCTGGGCTGGGCCTGACTGAGGC | |
| CATTGACAAGAACAAGGCCGACTTATCACGCATGTCTGGCAAGAAGGATCTGTACCTGGCCAGT | |
| GTGTTCCACGCCACCGCCTTTGAGTTGGACACAGATGGCAACCCCTTTGACCAGGACATCTACG | |
| GGCGCGAGGAGCTGCGCAGCCCCAAGCTGTTCTACGCCGACCACCCCTTCATCTTCCTGGTGC | |
| GGGACACCCAAAGCGGCTCCCTGCTATTCATTGGGCGCCTGGTCCGGCTCAAGGGTGACAAGAT | |
| GCGAGACGAGTTATAGGGCCTCAGGGTGCACACAGGATGGCAGGAGGCATCCAAAGGCTCCTG | |
| AGACACATGGGTGCTATTGGGGTTGGGGGGGAGGTGAGGTACCAGCCTTGGATACTCCATGGAA | |
| TTCGAGCTCCACTTGGACATGGGCCCCAGATACCATGATGCTGAGCCCGGAAACTCCACATCCT | |
| GTGGGACCTGGGCCATAGTCATTCTGCCTGCCCTGAAAGTCCCAGATCAAGCCTGCCTCAATCA | |
| GTATTCATATTTATAGCCAGGTACCTTCTCACCTGTGAGACCAAATTGAGCTCGGGGGGTCAGCC | |
| AGCCCTCTTCTGACACTAAAACACCTCAGCTGCCTCCCCAGCTCTATCCCAACCTCTCCCAACTA | |
| TAAAACTAGGTGCTGCAGCCTGGGACCAGGCACCCCCAGAATGACCTGGCCGCAGTGAGGCGA | |
| TTGAGAAGGAGGTCCCAGGAGGGGCTTCTGGGAAGACCCTGGTCAAGAAGCATCGTCTGGCGTT | |
| GTGGGGATGAACTTTTTGTTTTGTTTCTTCCTTTTTTAGTTCTTCAAGGAATGGGGGGCCAGGGG | |
| GGCAATGAGCCTTTGTTGCTAATCAAATCCGGGACTTGTTTGTACGTTTTTTTTTCTCACTGAAAC | |
| CTTTTCCAGTGCCAAAAAAAAA | |
| NM_005952: Metallothionein 1X(MT1X) | |
| ACCACGCTTTTCATCTGTCCCGCTGCGTGTTTTCCTCTTGATCGGGAACTCCTGCTTCTCCTTGCC | Seq. ID No. 130 |
| TCGAAATGGACCCCAACTGCTCCTGCTCGCCTGTTGGCTCCTGTGCCTGTGCCGGCTCCTGCAA | |
| ATGCAAAGAGTGCAAATGCACCTCCTGCAAGAAGAGCTGCTGCTCCTGCTGCCCTGTCGGCTGT | |
| GCCAAGTGTGCCCAGGGCTGCATCTGCAAAGGGACGTCAGACAAGTGCAGCTGCTGTGCCTGA | |
| XM_030707: KIAA0620 protein (KIAA0620) | |
| CCCCCCCCCCGCCTCCCGCCGCCTCCGGGCTCCCGGCTCCCGGCCGCGCCTCGCCCCATGCA | Seq. ID No. 131 |
| CTCGCCGCGCCGCGCAGCCCGCGCACGCCCGGATGGCTCCTCGCGCCGCGGGCGGCGCACCC | |
| CTTAGCGCCCGGGCCGCCGCCGCCAGCCCCCCGCCGTTCCAGACGCCGCCGCGGTGCCCGGT | |
| GCCGCTGCTGTTGCTGCTGCTCCTGGGGGCGGCGCGGGCCGGCGCCCTGGAGATCCAGCGTC | |
| GGTTCCCCTCGCCCACGCCCACCAACAACTTCGCCCTGGACGGCGCGGCGGGGACCGTGTACC | |
| TGGCGGCCGTCAACCGCCTCTATCAGCTGTCGGGCGCCAACCTGAGCCTGGAGGCCGAGGCGG | |
| CCGTGGGCCCGGTGCCCGACAGCCCGCTGTGTCACGCTCCGCAGCTGCCGCAGGCCTCGTGC | |
| GAGCACCCGCGGCGCCTCACGGACAACTACAACAAGATCCTGCAGCTGGACCCCGGCCAGGGC | |
| CTGGTAGTCGTGTGCGGGTCCATCTACCAGGGCTTCTGCCAGCTGCGGCGCCGGGGCAACATC | |
| TCGGCCGTGGCCGTGCGCTTCCCGCCCGCCGCGCCGCCCGCCGAGCCCGTCACGGTGTTCCC | |
| CAGCATGCTGAACGTGGCGGCCAACCACCCGAACGCGTCCACCGTGGGGCTAGTTCTGCCTCC | |
| CGCCGCGGGCGCGGGGGGCAGCCGCCTGCTCGTGGGCGCCACGTACACCGGTTACGGCAGCT | |
| CCTTCTTCCCGCGCAACCGCAGCCTGGAGGACCACCGCTTCGAGAACACGCCCGAGATCGCCAT | |
| CCGCTCCCTGGACACGCGCGGCGACCTGGCCAAGCTCTTCACCTTCGACCTCAACCCCTCCGAC | |
| GACAACATCCTCAAGATCAAGCAGGGCGCCAAGGAGCAGCACAAGCTGGGCTTCGTGAGCGCC | |
| TTCCTGCACCCGTCCGACCCGCCGCCGGGTGCACAGTCCTACGCGTACCTGGCGCTCAACAGC | |
| GAGGCGCGCGCGGGCGACAAGGAGAGCCAGGCGCGGAGCCTGCTGGCGCGCATCTGCCTGCC | |
| CCACGGCGCCGGCGGCGACGCCAAGAAGCTCACCGAGTCCTACATCCAGTTGGGCTTGCAGTG | |
| CGCGGGCGGCGCGGGCCGCGGCGACCTCTACAGCCGCCTGGTGTCGGTCTTCCCAGCCCGGG | |
| AGCGGCTCTTTGCTGTCTTCGAGCGGCCCCAGGGGTCCCCCGCGGCCCGCGCTGCTCCGGCCG | |
| CACTCTGCGCCTTCCGCTTCGCCGACGTGCGAGCCGCCATCCGAGCTGCGCGCACCGCCTGCT | |
| TCGTGGAACCGGCGCCCGACGTGGTGGCGGTGCTCGACAGCGTGGTGCAGGGCACGGGACCG | |
| GCCTGCGAGCGCAAGCTCAACATCCAGCTCCAGCCAGAGCAGCTGGACTGTGGAGCTGCTCAC | |
| CTGCAGCACCCGCTGTCCATCCTGCAGCCCCTGAAGGCCACGCCCGTGTTCCGCGCCCCGGGC | |
| CTCACCTCCGTGGCCGTGGCCAGCGTCAACAACTACACAGCGGTCTTCCTGGGCACGGTCAACG | |
| GGAGGCTTCTCAAGATCAACCTGAACGAGAGCATGCAGGTGGTGAGCAGGCGGGTGGTGACTG | |
| TGGCCTATGGGGAGCCCGTGCACCATGTCATGCAGTTTGACCCAGCAGACTCCGGTTACCTTTA | |
| CCTGATGACGTCCCACCAGATGGCCAGGGTGAAGGTCGCCGCCTGCAACGTGCACTCCACCTGT | |
| GGGGACTGCGTGGGTGCGGCGGACGCCTACTGCGGCTGGTGTGCCCTGGAGACGCGGTGCAC | |
| CTTGCAGCAGGACTGCACCAATTCCAGCCAGCAGCATTTCTGGACCAGTGCCAGCGAGGGCCCC | |
| AGCCGCTGTCCTGCCATGACCGTCCTGCCTTCCGAGATCGATGTGCGCCAGGAGTACCCAGGCA | |
| TGATCCTGCAGATCTCGGGCAGCCTGCCCAGCCTCAGTGGCATGGAGATGGCCTGTGACTATGG | |
| GAACAACATCCGCACTGTGGCTCGGGTCCCAGGCCCTGCCTTTGGTCACCAGATTGCCTACTGC | |
| AACCTCCTGCCGAGGGACCAGTTTCCGCCCTTCCCCCCCAACCAGGACCACGTGACTGTTGAGA | |
| TGTCTGTGAGGGTCAATGGGCGGAACATCGTCAAGGCCAATTTCACCATCTACGACTGCAGCCG | |
| CACTGCACAAGTGTACCCCCACACAGCCTGTACCAGCTGCCTGTCGGCACAGTGGCCCTGTTTC | |
| TGGTGCAGCCAGCAGCACTCCTGTGTTTGCAACCAGTCTCGGTGCGAGGCCTCACCAAACCCCA | |
| CGAGCCCTCAGGACTGCCCCCGGACCCTGCTCTCACCCCTGGCACCCGTGCCTACCGGTGGCT | |
| CCCAGAACATCCTGGTGCCTCTGGCCAACACTGCCTTTTTCCAGGGTGCAGCCCTGGAGTGTAG | |
| TTTTGGGCTGGAGGAGATCTTCGAGGCTGTGTGGGTGAATGAGTCTGTTGTACGCTGTGACCAG | |
| GTGGTGCTGCACACGACCCGGAAGAGCCAGGTGTTCCCGCTCAGCCTCCAACTAAAGGGGCGG | |
| CCAGCCCGATTCCTGGACAGCCCTGAGCCCATGACAGTCATGGTCTATAACTGTGCCATGGGCA | |
| GCCCCGACTGTTCCCAGTGCCTGGGCCGCGAAGACCTGGGTCACCTGTGCGTGTGGAGTGATG | |
| GCTGCCGCCTGCGGGGGCCTCTGCAGCCCATGGCTGGCACCTGCCCCGCCCCCGAGATCCGC | |
| GCGATTGAGCCCCTGAGTGGCCCGTTGGACGGTGGGACCCTGCTGACCATCCGAGGAAGGAAC | |
| CTGGGCCGGCGGCTCAGTGACGTGGCCCACGGCGTGTGGATTGGTGGTGTGGCCTGTGAGCCA | |
| CTGCCTGACAGATACACGGTGTCGGAGGAGATCGTGTGTGTCACAGGGCCAGCCCCAGGACCG | |
| CTCTCAGGTGTGGTGACCGTGAACGCCTCTAAGGAGGGCAAGTCCCGGGACCGCTTCTCCTACG | |
| TGCTGCCCCTGGTCCACTCCCTGGAGCCTACCATGGGCCCCAAGGCCGGGGGCACCAGGATCA | |
| CCATCCATGGGAATGACCTCCATGTAGGCTCCGAGCTCCAGGTCCTGGTGAACGACACAGACCC | |
| CTGCACGGAGCTGATGCGCACAGATACCAGCATCGCCTGCACCATGCCTGAGGGGGCCCTGCC | |
| GGCTCCGGTGCCTGTGTGTGTGCGCTTCGAGCGTCGGGGCTGCGTGCACGGCAACCTCACCTT | |
| CTGGTACATGCAGAACCCGGTCATCACGGCCATCAGTCCCCGCCGCAGCCCTGTCAGTGGCGG | |
| CAGGACCATCACAGTGGCTGGTGAGCGTTTCCACATGGTGCAGAATGTGTCCATGGCCGTCCAC | |
| CACATTGGCCGGGAGCCCACGCTCTGCAAGGTTCTCAACTCCACCCTCATCACCTGCCCGTCCC | |
| CCGGGGCCCTGAGCAACGCATCAGCGCCAGTGGACTTCTTCATCAATGGGCGGGCCTACGCAG | |
| ACGAGGTGGCTGTGGCTGAGGAGCTACTGGACCCCGAGGAGGCACAGCGGGGCAGCAGGTTC | |
| CGCCTGGACTACCTCCCCAACCCACAGTTCTCTACGGCCAAGAGGGAGAAGTGGATCAAGCACC | |
| ACCCCGGGGAGCCTCTCACCCTCGTTATCCACAAGGAGCAGGACAGCCTGGGGCTCCAGAGTC | |
| ACGAGTACCGGGTCAAGATAGGCCAAGTAAGCTGCGACATCCAGATTGTCTCTGACAGAATCATC | |
| CACTGCTCGGTCAACGAGTCCCTGGGCGCGGCCGTGGGGCAGCTGCCCATCACAATCCAGGTA | |
| GGGAACTTCAACCAGACCATCGCCACACTGCAGCTGGGGGGCAGCGAGACGGCCATCATCGTG | |
| TCCATCGTCATCTGCAGCGTCCTGCTGCTGCTCTCCGTGGTGGCCCTGTTCGTCTTTTGTACCAA | |
| GAGCCGACGTGCTGAGCGTTACTGGCAGAAGACGCTGCTGCAGATGGAGGAGATGGAATCTCA | |
| GATCCGAGAGGAAATCCGCAAAGGCTTCGCTGAGCTGCAGACAGACATGACAGATCTGACCAAG | |
| GAGCTGAACCGCAGCCAGGGCATCCCCTTCCTGGAGTATAAGCACTTCGTGACCCGCACCTTCT | |
| TCCCCAAGTGTTCCTCCCTTTATGAAGAGCGTTACGTGCTGCCCTCCCAGACCCTCAACTCCCAG | |
| GGCAGCTCCCAGGCACAGGAAACCCACCCACTGCTGGGAGAGTGGAAGATTCCTGAGAGCTGC | |
| CGGCCCAACATGGAAGAGGGAATTAGCTTGTTCTCCTCACTACTCAACAACAAGCACTTCCTCAT | |
| CGTCTTTGTCCACGCGCTGGAGCAGCAGAAGGACTTTGCGGTGCGCGACAGGTGCAGCCTGGC | |
| CTCGCTGCTGACCATCGCGCTGCACGGCAAGCTGGAGTACTACACCAGCATCATGAAGGAGCTG | |
| CTGGTGGACCTCATTGACGCCTCGGCCGCCAAGAACCCCAAGCTCATGCTGCGGCGCACAGAG | |
| TCTGTGGTGGAGAAGATGCTCACCAACTGGATGTCCATCTGCATGTACAGCTGTCTGCGGGAGA | |
| CGGTGGGGGAGCCATTCTTCCTGCTGCTGTGTGCCATCAAGCAGCAAATCAACAAGGGCTCCAT | |
| CGACGCCATCACAGGCAAGGCCCGCTACACACTCAGTGAGGAGTGGCTGCTGCGGGAGAACAT | |
| CGAGGCCAAGCCCCGGAACCTGAACGTGTCCTTCCAGGGCTGTGGCATGGACTCGCTGAGCGT | |
| GCGGGCCATGGACACCGACACGCTGACACAGGTCAAGGAGAAGATCCTGGAGGCCTTCTGCAA | |
| GAATGTGCCCTACTCCCAGTGGCCGCGTGCAGAGGACGTCGACCTTGGTGGTTCGCCTCCAGCA | |
| CACAGAGCTACATCCTTCGGGACCTGGACGACACCTCAGTGGTGGAAGACGGCCGCAAGAAGCT | |
| TAACACGCTGGCCCATTACAAGATCCCTGAAGGTGCCTCCCTGGCCATGAGTCTCATAGACAAGA | |
| AGGACAACACACTGGGCCGAGTGAAAGACTTGGACACAGAGAAGTATTTCCATTTGGTGCTGCCT | |
| ACGGACGAGCTGGCGGAGCCCAAGAAGTCTCACCGGCAGAGCCATCGCAAGAAGGTGCTCCCG | |
| GAAATCTACCTGACCCGCCTGCTCTCCACCAAGGGCACGTTGCAGAAGTTTCTGGATGACCTGTT | |
| CAAGGCCATTCTGAGTATCCGTGAAGACAAGCCCCCACTGGCTGTCAAGTACTTTTTCGACTTCC | |
| TGGAGGAGCAGGCTGAGAAGAGGGGAATCTCCGACCCCGACACCCTACACATCTGGAAGACCA | |
| ACAGCCTTCCTCTCCGGTTCTGGGTGAACATCCTGAAGAACCCCCAGTTTGTCTTTGACATCGAC | |
| AAGACAGACCACATCGACGCCTGCCTTTCAGTCATCGCGCAGGCCTTCATCGACGCCTGCTCCA | |
| TCTCTGACCTGCAGCTGGGCAAGGATTCGCCAACCAACAAGCTCCTCTACGCCAAGGAGATTCC | |
| TGAGTACCGGAAGATCGTGCAGCGCTACTACAAGCAGATCCAGGACATGACGCCGCTCAGCGAG | |
| CAAGAGATGAATGCCCATCTGGCCGAGGAGTCGAGGAAATACCAGAATGAGTTCAACACCAATG | |
| TGGCCATGGCAGAGATTTATAAGTACGCCAAGAGGTATCGGCCGCAGATCATGGCCGCGCTGGA | |
| GGCCAACCCCACGGCCCGGAGGACACAACTGCAGCACAAGTTTGAGCAGGTGGTGGCTTTGAT | |
| GGAGGACAACATCTACGAGTGCTACAGTGAGGCCTGAGAGACATGGAGAGTTGGTCAGGCTGCT | |
| GCTGGGAGAAATGGACGCCCACTGGGCCTCAACTTGATCTTCTACCCCGTGCCTGTGACTCAGA | |
| CTGGGAAATACTGAGCAGAGACGGCTGGGGCGGGGGCAGGAGGAGGGGCTGCTCTCTGAGAC | |
| AGGGGCGCCCCCGCCTTGACCCCTGGGCACCTCCATCCCCTCCCACCTGTCCCCAGATCAGTCT | |
| CTGGGATGGAGGCGAGAGAGCTGGTCAGGCTCCCCCATCTGCCCAGCACGGCCTGCACTGTGC | |
| CCACCCACTTGCTCCACAACGTCCAGTTGGTCCTGCTGCCAAGAGCCCCGTGCATCCAGGCGGC | |
| CAAGCACAAACTGGGGGAGAGGAGGCCGCCAGCCCGGAGGCTGCAGCCCAGAAACTCTACCTC | |
| ATCCACACTGGTGCAGGGAGCCCTCCTTGAACTGACCTTTGATTGGTTTCTGCTTCAACTACCAA | |
| AATGTTATCTCCACTTCCCCCTCACCCGTAGAGGATCCTGGCCACAGACAGTTTCAAGTAGTGTC | |
| AGATTTTTGTTGCTTGGGCGGCTGTTGGTAGAGTGGGCAGTGCCCGCGCCATGGGGTGCTCTGT | |
| GGGCTTCTCCAGGAGCAGGGAGGGTGGAGGGGAGGGATGGGGGGCACAGGAGCTGGGAGCC | |
| CCGTCTCCAGGAAAAGGAGAGGGGTTAAGATGCACCGAGGCTGTAGCTGGGCTACTTGATCTTG | |
| CTGAAAGTGTTTCTAAAGATAGCACCACTTTTTTTTTTAAAGCTTTTATATATTAAAAAACGTATCAT | |
| GCACCAACTGTGAATAGCTGCCGCTTGCGCAGAGGACCCGGGGAGGGGTCCCGAGAGGCTCCC | |
| CATGCAACACTGGAAATGACTGTTCCAGAGAGCGGGCAGACCTGGCAGAGCGCCCCTGGCGCC | |
| TGAGACTACCACCCACTCCGTTCCTGCCAGAAACGACCCTCTGTGGCCGATGGGCCATGCGGGC | |
| CCCTCGCAGCCAACTCAGCCAGTGTTGGGACTGGCTCAGAGCCCATGGGGGCTGGAGGGGGGC | |
| AGCTGGGACTCTGGAATCTTCTTTATAATAAAAGCCTTACGG | |
| AC | |
| NM_003254: Tissue inhibitor of metalloproteinase 1 (TIMP1) | |
| AGGGGCCTTAGCGTGCCGCATCGCCGAGATCCAGCGCCCAGAGAGACACCAGAGAACCCACCA | Seq. ID No. 132 |
| TGGCCCCCTTTGAGCCCCTGGCTTCTGGCATCCTGTTGTTGCTGTGGCTGATAGCCCCCAGCAG | |
| GGCCTGCACCTGTGTCCCACCCCACCCACAGACGGCCTTCTGCAATTCCGACCTCGTCATCAGG | |
| GCCAAGTTCGTGGGGACACCAGAAGTCAACCAGACCACCTTATACCAGCGTTATGAGATCAAGAT | |
| GACCAAGATGTATAAAGGGTTCCAAGCCTTAGGGGATGCCGCTGACATCCGGTTCGTCTACACC | |
| CCCGCCATGGAGAGTGTCTGCGGATACTTCCACAGGTCCCACAACCGCAGCGAGGAGTTTCTCA | |
| TTGCTGGAAAACTGCAGGATGGACTCTTGCACATCACTACCTGCAGTTTCGTGGCTCCCTGGAAC | |
| AGCCTGAGCTTAGCTCAGCGCCGGGGCTTCACCAAGACCTACACTGTTGGCTGTGAGGAATGCA | |
| CAGTGTTTCCCTGTTTATCCATCCCCTGCAAACTGCAGAGTGGCACTCATTGCTTGTGGACGGAC | |
| CAGCTCCTCCAAGGCTCTGAAAAGGGCTTCCAGTCCCGTCACCTTGCCTGCCTGCCTCGGGAGC | |
| CAGGGCTGTGCACCTGGCAGTCCCTGCGGTCCCAGATAGCCTGAATCCTGCCCGGAGTGGAAC | |
| TGAAGCCTGCACAGTGTCCACCCTGTTCCCACTCCCATCTTTCTTCCGGACAATGAAATAAAGAG | |
| TTACCACCCAGC | |
| NM_006185: Nuclear mitotic apparatus protein 1 (NUMA1) | |
| GCCCACGAAGAGGTACGATTCCGGAGAATCGCGAGGCAGAGCGGGAGCGCGCAGCCAGGTGG | Seq. ID No. 133 |
| AAACTAATTCTAAGCCAGACTGCTGGAGATCACCCTGTTCTAGTGTGTGGAGGCTTCCACCAGGA | |
| GTCTGGAGTGCAATGGCACGATCTCGGCTCACTGCAACCTCCACCTCCCAGGTTCAAGCGATTC | |
| TCCTGCCTCAGCCTCCCAAGTAGCTGGGATTACAGGCGCATTGGAGTGACTGTCTGGCATCACC | |
| AAGATGACACTCCACGCCACCCGGGGGGCTGCACTCCTCTCTTGGGTGAACAGTCTACACGTGG | |
| CTGACCCTGTGGAGGCTGTGCTGCAGCTCCAGGACTGCAGCATCTTCATCAAGATCATTGACAG | |
| AATCCATGGCACTGAAGAGGGACAGCAAATCTTGAAGCAGCCGGTGTCAGAGAGACTGGACTTT | |
| GTGTGCAGTTTTCTGCAGAAAAATCGAAAACATCCCTCTTCCCCAGAATGCCTGGTATCTGCACA | |
| GAAGGTGCTAGAGGGATCAGAGCTGGAACTGGCGAAGATGACCATGCTGCTCTTATACCACTCT | |
| ACCATGAGCTCCAAAAGTCCCAGGGACTGGGAACAGTTTGAATATAAAATTCAGGCTGAGTTGGC | |
| TGTCATTCTTAAATTTGTGCTGGACCATGAGGACGGGCTAAACCTTAATGAGGACCTAGAGAACT | |
| TCCTACAGAAAGCTCCTGTGCCTTCTACCTGTTCTAGCACATTCCCTGAAGAGCTCTCCCCACCT | |
| AGCCACCAGGCCAAGAGGGAGATTCGCTTCCTAGAGCTACAGAAGGTTGCCTCCTCTTCCAGTG | |
| GGAACAACTTTCTCTCAGGTTCTCCAGCTTCTCCCATGGGTGATATCCTGCAGACCCCACAGTTC | |
| CAGATGAGACGGCTGAAGAAGCAGCTTGCTGATGAGAGAAGTAATAGGGATGAGCTGGAGCTGG | |
| AGCTAGCTGAGAACCGCAAGCTCCTCACCGAGAAGGATGCACAGATAGCCATGATGCAGCAGCG | |
| CATTGACCGCCTAGCCCTGCTGAATGAGAAGCAGGCGGCCAGCCCACTGGAGCCCAAGGAGCT | |
| TGAGGAGCTGCGTGACAAGAATGAGAGCCTTACCATGCGGCTGCATGAAACCCTGAAGCAGTGC | |
| CAGGACCTGAAGACAGAGAAGAGCCAGATGGATCGCAAAATCAACCAGCTTTCGGAGGAGAATG | |
| GAGACCTTTCCTTTAAGCTGCGGGAGTTTGCCAGTCATCTGCAGCAGCTACAGGATGCCCTCAAT | |
| GAGCTGACGGAGGAGCACAGCAAGGCCACTCAGGAGTGGCTAGAGAAGCAGGCCCAGCTGGAG | |
| AAGGAGCTCAGCGCAGCCCTGCAGGACAAGAAATGCCTTGAAGAGAAGAACGAAATCCTTCAGG | |
| GAAAACTTTCACAGCTGGAAGAACACTTGTCCCAGCTGCAGGATAACCCACCCCAGGAGAAGGG | |
| CGAGGTGCTGGGTGATGTCTTGCAGCTGGAAACCTTGAAGCAAGAGGCAGCCACTCTTGCTGCA | |
| AACAACACACAGCTCCAAGCCAGGGTAGAGATGCTGGAGACTGAGCGAGGCCAGCAGGAAGCC | |
| AAGCTGCTTGCTGAGCGGGGCCACTTCGAAGAAGAAAAGCAGCAGCTGTCTAGCCTGATCACTG | |
| ACCTGCAGAGCTCCATCTCCAACCTCAGCCAGGCCAAGGAAGAGCTGGAGCAGGCCTCCCAGG | |
| CTCATGGGGCCCGGTTGACTGCCCAGGTGGCCTCTCTGACCTCTGAGCTCACCACACTCAATGC | |
| CACCATCCAGCAACAGGATCAAGAACTGGCTGGCCTGAAGCAGCAGGCCAAAGAGAAGCAGGC | |
| CCAGCTAGCACAGACCCTCCAACAGCAAGAACAGGCCTCCCAGGGCCTCCGCCACCAGGTGGA | |
| GCAGCTAAGCAGTAGCCTGAAGCAGAAGGAGCAGCAGTTGAAGGAGGTAGCGGAGAAGCAGGA | |
| GGCAACTAGGCAGGACCATGCCCAGCAACTGGCCACTGCTGCAGAGGAGCGAGAGGCCTCCTT | |
| AAGGGAGCGGGATGCGGCTCTCAAGCAGCTGGAGGCACTGGAGAAGGAGAAGGCTGCCAAGCT | |
| GGAGATTCTGCAGCAGCAACTTCAGGTGGCTAATGAAGCCCGGGACAGTGCCCAGACCTCAGTG | |
| ACACAGGCCCAGCGGGAGAAGGCAGAGCTGAGCCGGAAGGTGGAGGAACTCCAGGCCTGTGTT | |
| GAGACAGCCCGCCAGGAACAGCATGAGGCCCAGGCCCAGGTTGCAGAGCTAGAGTTGCAGCTG | |
| CGGTCTGAGCAGCAAAAAGCAACTGAGAAAGAAAGGGTGGCCCAGGAGAAGGACCAGCTCCAG | |
| GAGCAGCTCCAGGCCCTCAAAGAGTCCTTGAAGGTCACCAAGGGCAGCCTTGAAGAGGAGAAG | |
| CGCAGGGCTGCAGATGCCCTGGAAGAGCAGCAGCGTTGTATCTCTGAGCTGAAGGCAGAGACC | |
| CGAAGCCTGGTGGAGCAGCATAAGCGGGAACGAAAGGAGCTGGAAGAAGAGAGGGCTGGGCG | |
| CAAGGGGCTGGAGGCTCGATTACTGCAGCTTGGGGAGGCCCATCAGGCTGAGACTGAAGTCCT | |
| GCGGCGGGAGCTGGCAGAGGCCATGGCTGCCCAGCACACAGCTGAGAGTGAGTGTGAGCAGCT | |
| CGTCAAAGAAGTAGCTGCCTGGCGTGACGGGTATGAGGATAGCCAGCAAGAGGAGGCACAGTAT | |
| GGCGCCATGTTCCAGGAACAGCTGATGACTTTGAAGGAGGAATGTGAGAAGGCCCGCCAGGAG | |
| CTGCAGGAGGCAAAGGAGAAGGTGGCAGGCATAGAATCCCACAGCGAGCTCCAGATAAGCCGG | |
| CAGCAGAACAAACTAGCTGAGCTCCATGCCAACCTGGCCAGAGCACTCCAGCAGGTCCAAGAGA | |
| AGGAAGTCAGGGCCCAGAAGCTTGCAGATGACCTCTCCACTCTGCAGGAAAAGATGGCTGCCAC | |
| CAGCAAAGAGGTGGCCCGCTTGGAGACCTTGGTGCGCAAGGCAGGTGAGCAGCAGGAAACAGC | |
| CTCCCGGGAGTTAGTCAAGGAGCCTGCGAGGGCAGGAGACAGACAGCCCGAGTGGCTGGAAGA | |
| GCAACAGGGACGCCAGTTCTGCAGCACACAGGCAGCGCTGCAGGCTATGGAGCGGGAGGCAGA | |
| GCAGATGGGCAATGAGCTGGAACGGCTGCGGGCCGCGCTGATGGAGAGCCAGGGGCAGCAGC | |
| AGGAGGAGCGTGGGCAGCAGGAAAGGGAGGTGGCGCGGCTGACCCAGGAGCGGGGCCGTGC | |
| CCAGGCTGACCTTGCCCTGGAGAAGGCGGCCAGAGCAGAGCTTGAGATGCGGCTGCAGAACGC | |
| CCTCAACGAGCAGCGTGTGGAGTTCGCTACCCTGCAAGAGGCACTGGCTCATGCCCTGACGGAA | |
| AAGGAAGGCAAGGACCAGGAGTTGGCCAAGCTTCGTGGTCTGGAGGCAGCCCAGATAAAAGAG | |
| CTGGAGGAACTTCGGCAAACCGTGAAGCAACTGAAGGAACAGCTGGCTAAGAAAGAAAAGGAGC | |
| ACGCATCTGGCTCAGGAGCCCAATCTGAGGCTGCTGGCAGGACAGAGCCAACAGGCCCCAAGC | |
| TGGAAGCACTGCGGGCAGAGGTGAGCAAGCTGGAACAGCAATGCCAGAAGCAGCAGGAGCAGG | |
| CTGACAGCCTGGAACGCAGCCTCGAGGCTGAGCGGGCCTCCCGGGCTGAGCGGGACAGTGCT | |
| CTGGAGACTCTGCAGGGCCAGTTAGAGGAGAAGGCCCAGGAGCTAGGGCACAGTCAGAGTGCC | |
| TTAGCCTCGGCCCAACGGGAGTTGGCTGCCTTCCGCACCAAGGTACAAGACCACAGCAAGGCTG | |
| AAGATGAGTGGAAGGCCCAGGTGGCCCGGGGCCGGCAAGAGGCTGAGAGGAAAAATAGCCTCA | |
| TCAGCAGCTTGGAGGAGGAGGTGTCCATCCTGAATCGCCAGGTCCTGGAGAAGGAGGGGGAGA | |
| GCAAGGAGTTGAAGCGGCTGGTGATGGCCGAGTCAGAGAAGAGCCAGAAGCTGGAGGAGAGCT | |
| GCGCCTGCTGCAGGCAGAGACAGCCAGCAACAGTGCCAGAGCTGCAGAACGCAGCTCTGCTCT | |
| GCGGGAGGAGGTGCAGAGCCTCCGGGAGGGAGGCTGAGAAACAGCGGGTGGCTTCAGAGAAC | |
| CTGCGGCAGGAGCTGACCTCACAGGCTGAGCGTGCGGAGGAGCTGGGCCAAGAATTGAAGGCG | |
| TGGCAGGAGAAGTTCTTCCAGAAAGAGCAGGCCCTCTCCACCCTGCAGCTCGAGCACACCAGCA | |
| CACAGGCCCTGGTGAGTGAGCTGCTGCCAGCTAAGCACCTCTGCCAGCAGCTGCAGGCCGAGC | |
| AGGCCGCTGCCGAGAAACGCCACCGTGAGGAGCTGGAGCAGAGCAAGCAGGCCGCTGGGGGA | |
| CTGCGGGCAGAGCTGCTGCGGGCCCAGCGGGAGCTTGGGGAGCTGATTCCTCTGCGGCAGAA | |
| GGTGGCAGAGCAGGAGCGAACAGCTCAGCAGCTGCGGGCAGAGAAGGCCAGCTATGCAGAGCA | |
| GCTGAGCATGCTGAAGAAGGCGCATGGCCTGCTGGCAGAGGAGAACCGGGGGCTGGGTGAGC | |
| GGGCCAACCTTGGCCGGCAGTTTCTGGAAGTGGAGTTGGACCAGGCCCGGGAAAAGTATGTCC | |
| AAGAGTTGGCAGCCGTACGTGCTGATGCTGAGACCCGTCTGGCTGAGGTGCAGCGAGAAGCAC | |
| AGAGCACTGCCCGGGAGCTGGAGGTGATGACTGCCAAGTATGAGGGTGCCAAGGTCAAGGTCC | |
| TGGAGGAGAGGCAGCGGTTCCAGGAAGAGAGGCAGAAACTCACTGCCCAGGTGGAAGAACTGA | |
| GTAAGAAACTGGCTGACTCTGACCAAGCCAGCAAGGTGCAGCAGCAGAAGCTGAAGGCTGTCCA | |
| GGCTGAGGGAGGCGAGAGCCAGCAGGAGGCCCAGCGCTTCCAGGCCCAGCTGAATGAACTGCA | |
| AGCCCAGTTGAGCCAGAAGGAGCAGGCAGCTGAGCACTATAAGCTGCAGATGGAGAAAGCCAAA | |
| ACACATTATGATGCCAAGAAGCAGCAGAACCAAGAGCTGCAGGAGCAGCTGCGGAGCCTGGAG | |
| CAGCTGCAGAAGGAAAACAAAGAGCTGCGAGCTGAAGCTGAACGGCTGGGCCATGAGCTACAG | |
| CAGGCTGGGCTGAAGACCAAGGAGGCTGAACAGACCTGCCGCCACCTTACTGCCCAGGTGCGC | |
| AGCCTGGAGGCACAGGTTGCCCATGCAGACCAGCAGCTTCGAGACCTGGGCAAATTCCAGGTG | |
| GCAACTGATGCTTTAAAGAGCCGTGAGCCCCAGGCTAAGCCCCAGCTGGACTTGAGTATTGACA | |
| GCCTGGATCTGAGCTGCGAGGAGGGGACCCCACTCAGTATCACCAGCAAGCTGCCTCGTACCCA | |
| GCCAGACGGCACCAGCGTCCCTGGAGAACCAGCCTCACCTATCTCCCAGCGCCTGCCCCCCAA | |
| GGTAGAATCCCTGGAGAGTCTCTACTTCACTCCCATCCCTGCTCGGAGTCAGGCCCCCCTGGAG | |
| AGCAGCCTGGACTCCCTGGGAGACGTCTTCCTGGACTCGGGTCGTAAGACCCGCTCCGCTCGTC | |
| GGCGCACCACGCAGATCATCAACATCACCATGACCAAGAAGCTAGATGTGGAAGAGCCAGACAG | |
| CGCCAACTCATCGTTCTACAGCACGCGGTCTGCTCCTGCTTCCCAGGCTAGCCTGCGAGCCACC | |
| TCCTCTACTCAGTCTCTAGCTCGCCTGGGTTCTCCCGATTATGGCAACTCAGCCCTGCTCAGCTT | |
| GCCTGGCTACCGCCCCACCACTCGCAGTTCTGCTCGTCGTTCCCAGGCCGGGGTGTCCAGTGG | |
| GGCCCCTCCAGGAAGGAACAGCTTCTACATGGGCACTTGCCAGGATGAGCCTGAGCAGCTGGAT | |
| GACTGGAACCGCATTGCAGAGCTGCAGCAGCGCAATCGAGTGTGCCCCCCACATCTGAAGACCT | |
| GCTATCCCCTGGAGTCCAGGCCTTCCCTGAGCCTGGGCACCATCACAGATGAGGAGATGAAAAC | |
| TGGAGACCCCCAAGAGACCCTGCGCCGAGCCAGCATGCAGCCAATCCAGATAGCCGAGGGCAC | |
| TGGCATCACCACCCGGCAGCAGCGCAAACGGGTCTCCCTAGAGCCCCACCAGGGCCCTGGAAC | |
| TCCTGAGTCTAAGAAGGCCACCAGCTGTTTCCCACGCCCCATGACTCCCCGAGACCGACATGAA | |
| GGGCGCAAACAGAGCACTACTGAGGCCCAGAAGAAAGCAGCTCCAGCTTCTACTAAACAGGCTG | |
| ACCGGCGCCAGTCGATGGCCTTCAGCATCCTCAACACACCCAAGAAGCTAGGGAACAGCCTTCT | |
| GCGGCGGGGAGCCTCAAAGAAGGCCCTGTCCAAGGCTTCCCCCAACACTCGCAGTGGAACCCG | |
| CCGTTCTCCGCGCATTGCCACCACCACAGCCAGTGCCGCCACTGCTGCCGCCATTGGTGCCACC | |
| CCTCGAGCCAAGGGCAAGGCAAAGCACTAAAGGGCCAGTACCAGTGAGTGGCCCCACCTGTGT | |
| CCCCGATGCTGCCGTCACCTGGTCCTCCGCCTACTGTCCCTCTCAGTGCCTTCTCTCAGCTCCCA | |
| GGCCAACAGTAGCCAAACCCCTAGAGACAGTGATGCCTGCCCGCACCCTGGCCTGGCCCCTGG | |
| TCCTTCACTGGCGCCTTCTCGGAGCTGGCCCAGGGGGCCTGGAGCATGGACAGTGTGGGCGCT | |
| CTCCCTACCTTGCCTCCTTTTTCTTAAAGCAAAGTCACTTCTCCATCACAACCAGATTTGAGGCT | |
| GGTTTTGATGGCTGGGTCCTTGGGCCTGGCCAGTCTTCCTCTTAGCCTCTGGATCTAGAAGGGA | |
| CCATAAGAGGAGTAGGCCCTGGTTCCTGCTGTCCTGGTGGCTGGGCCAGCAGGGGCCCTCACT | |
| CTTGAAGTCCAGGACTGGGTCTGACCTGGTGGGAGCACCTGCCAGAGGATGCTCTTTCCCAGGA | |
| CGGATGGGCCCTGTGTCTCAGGAGTGGGGTTGGGGGACAGCCTTCAGCAGCAGCTCACACCCT | |
| ACCTTCCCCAGACTTGCACTGGGGTGGGATTTGGAGTGATGGGAAGGTTTTTAAGGGCCGGGGA | |
| TGGATCTTTTCTAAATGTTATTACTTGTAAATAAAGTCTATTTTT | |
| NM_004083: DNA-damage-inducible transcript 3 (DDIT3) | |
| GGCACGAGGGAGAGAGAGAGACTTAAGTCTAAGGCACTGAGCGTATCATGTTAAAGATGAGCGG | Seq. ID No. 134 |
| GTGGCAGCGACAGAGCCAAAATCAGAGCTGGAACCTGAGGAGAGAGTGTTCAAGAAGGAAGTGT | |
| ATCTTCATACATCACCACACCTGAAAGCAGATGTGCTTTTCCAGACTGATCCAACTGCAGAGATG | |
| GCAGCTGAGTCATTGCCTTTCTCCTTTGGGACACTGTCCAGCTGGGAGCTGGAAGCCTGGTATG | |
| AGGACCTGCAAGAGGTCCTGTCTTCAGATGAAAATGGGGGTACCTATGTTTCACCTCCTGGAAAT | |
| GAAGAGGAAGAATCAAAAATCTTCACCACTCTTGACCCTGCTTCTCTGGCTTGGCTGACTGAGGA | |
| GGAGCCAGAACCAGCAGAGGTCACAAGCACCTCCCAGAGCCCTCACTCTCCAGATTCCAGTCAG | |
| AGCTCCCTGGCTCAGGAGGAAGAGGAGGAAGACCAAGGGAGAACCAGGAAACGGAAACAGAGT | |
| GGTCATTCCCCAGCCCGGGCTGGAAAGCAGCGCATGAAGGAGAAAGAACAGGAGAATGAAAGG | |
| AAAGTGGCACAGCTAGCTGAAGAGAATGAACGGCTCAAGCAGGAAATCGAGCGCCTGACCAGG | |
| GAAGTAGAGGCGACTCGCCGAGCTCTGATTGACCGAATGGTGAATCTGCACCAAGCATGAACAA | |
| TTGGGAGCATCAGTCCCCCACTTGGGCCACACTACCCACCTTTCCCAGAAGTGGCTACTGACTAC | |
| CCTCTCACTAGTGCCAATGATGTGACCCTCAATCCCACATACGCAGGGGGAAGGCTTGGAGTAG | |
| ACAAAAGGAAAGGTCTCAGCTTGTATATAGAGATTGTACATTTATTTATTACTGTCCCTATCTATTA | |
| AAGTGACTTTCTATGAAAAAAAAAAAAAAAAAAAAAAAAAAMAAAAAAAAAAAAAAAAAAAAAA | |
| NM_016272: Transducer of ERBB2 (TOB2) | |
| ACTGGGGCCCACAGTCAGACATGAGCCACTGGTGGGACAGAAATAGGCTCCTGGTTCTGTGTGA | Seq. ID No. 135 |
| TCCANAGTTGGTGCTTTTCTGTCTATCCCTAGCTGTTGGTCACCACCAGCTTTCTGCATATTTTCT | |
| CACGGTGCCTCTCATTTCCCAGAGCCGCCTGGAGCCCAAGGCTGTACACGTGCCCTGTGCTGAT | |
| TCTCTGCCTAGGAAAGGACCATGCAGCTAGAGATCAAAGTGGCCCTGAACTTCATCATCTCCTAC | |
| TTGTACAACAAGCTGCCCCGGCGCCGGGCAGACCTGTTTGGGGAGGAGCTAGAGCGGCTTTTG | |
| AAAAAGAAATATGAAGGCCACTGGTACCCTGAGAAGCCACTGAAAGGCTCTGGCTTCCGCTGTGT | |
| TCACATTGGGGAGATGGTGGACCCCGTGGTGGAGCTGGCCGCCAAGCGGAGTGGCCTGGCAGT | |
| GGAAGATGTGCGGGCCAATGTGCCTGAGGAGCTGAGTGTCTGGATTGATCCCTTTGAGGTGTCC | |
| TACCAGATTGGTGAGAAGGGAGCTGTGAAAGTGCTGTACCTGGATGACAGTGAGGGTTGCGGTG | |
| CCCCAGAGCTGGACAAGGAGATCAAGAGCAGCTTCAACCCTGACGCCCAGGTGTTCGTGCCCAT | |
| TGGCAGCCAGGACAGCTCCCTGTCCAACTCCCCATCGCCATCCTTTGGCCAGTCACCCAGCCCT | |
| ACCTTCATTCCCCGCTCCGCTCAGCCCATCACCTTCACCACCGCCTCCTTCGCTGCCACCAAATT | |
| TGGCTCCACTAAGATGAAGAAGGGGGGCGGGGCAGCAAGTGGTGGGGGTGTAGCCAGCAGTG | |
| GGGCGGGTGGCCAGCAGCCACGACAGCAGCCTCGCATGGCCCGCTCACCCACCAACAGCCTGC | |
| TGAAGCACAAGAGCCTCTCTCTGTCTATGCATTCACTGAACTTCATCACGGCCAACCCGGCCCCT | |
| CAGTCCCAGCTCTCACCCAATGCCAAGGAGTTCGTGTACAACGGTGGTGGCTCACCCAGCCTCT | |
| TCTTTGATGCGGCCGATGGCCAGGGCAGCGGCACCCCAGGCCCGTTTGGAGGCAGTGGGGCTG | |
| GCACCTGCAACAGCAGCAGCTTTGACATGGCCCAGGTATTTGGAGGTGGTGCCAACAGCCTCTT | |
| CCTGGAGAAGACACCCTTTGTGGAAGGCCTCAGCTACAACCTGAACACCATGCAGTATCCCAGC | |
| CAGCAGTTCCAGCCCGTGGTGCTGGCCAACTGACCATCTACCTGCCCGTGGGGCCAGGAGCAC | |
| CCAAGACCACAGAAAAGAGAAAGGAAAGGCCAAAAAAAAGAGGAAAAGAAAAAAAAAAAAAA | |

Amino acid sequences

| | |
|---|---|
| NM_002982: Small inducible cytokineA2 (SCYA2) | |
| MKVSAALLCLLLIAATFIPQGLAQPDAINAPVTCCYNFTNRKISVQRLASYRRITSSKCP | Seq. ID No. 136 |
| KEAVIFKTIVAKEICADPKQKWVQDSMDHLDKQTQTPKT | |
| NM_015675: Growth arrest and DNA-damage-inducible beta (GADD45B) | |
| MTLEELVACDNAAQKMQTVTAAVEELLVAAQRQDRLTVGVYESAKLMNVDPDSVVLCLLA | Seq. ID No. 137 |
| IDEEEEDDIALQIHFTLIQSFCCDNDINIVRVSGNARLAQLLGEPAETQGTTEARDLHCL | |
| PFLQNPHTDAWKSHGLVEVASYCEESRGNNQWVPYISLQER | |
| NM_002964: S100 calcium binding protein A8 (S100A8) | |
| MLTELEKALNSIIDVYHKYSLIKGNFHAVYRDDLKKLLETECPQYIRKKGADVWFKELDI | Seq. ID No. 138 |
| NTDGAVNFQEFLILVIKMGVAAHKKSHEESHKE | |
| NM_078467: Cyclin-dependent kinase inhibitor 1A p21/Cip1 (CDKN1A) | |
| MSEPAGDVRQNPCGSKACRRLFGPVDSEQLSRDCDALMAGCIQEARERWNFDFVTETPLE | Seq. ID No. 139 |
| GDFAWERVRGLGLPKLYLPTGPRRGRDELGGGRRPGTSPALLQGTAEEDHVDLSLSCTLV | |
| PRSGEQAEGSPGGPGDSQGRKRRQTSMTDFYHSKRRLIFSKRKP | |
| NM_016232; Interleukin 1receptor-like 1 (IL1RL1) | |
| MGFWILAILTILMYSTAAKFSKQSWGLENEALIVRCPRQGKPSYTVDWYYSQTNKSIPTQ | Seq. ID No. 140 |
| ERNRVFASGQLLKFLPAEVADSGIYTCIVRSPTFNRTGYANVTIYKKQSDCNVPDYLMYS | |
| TVSGSEKNSKIYCPTIDLYNWTAPLEWFKNCQALQGSRYRAHKSFLVIDNVMTEDAGDYT | |
| CKFIHNENGANYSVTATRSFTVKDEQGFSLFPVIGAPAQNEIKEVEIGKNANLTCSACFG | |
| KGTQFLAAVLWQLNGTKITDFGEPRIQQEEGQNQSFSNGLACLDMVLRIADVKEEDLLLQ | |
| YDCLALNLHGLRRHTVRLSRKNPIDHHSIYCIIAVCSVFLMLINVLVIILKMFWIEATLL | |
| WRDIAKPYKTRNDGKLYDAYVVYPRNYKSSTDGASRVEHFVHQILPDVLENKCGYTLCIY | |
| GRDMLPGEDVVTAVETNIRKSRRHIFILTPQITHNKEFAYEQEVALHCALIQNDAKVILI | |
| EMEALSELDMLQAEALQDSLQHLMKVQGTIKWREDHIANKRSLNSKFWKHVRYQMPVPSK | |
| IPRKASSLTPLAAQKQ | |
| NM_004613: Transglutaminase 2 (TGM2) | |
| MAEELVLERCDLELETNGRDHHTADLCREKLVVRRGQPFWLTLHFEGRNYQASVDSLTFSVVTGPAPSQEAGTKA | Seq ID No. 141 |
| RFPLRDAVEEGDWTATVVDQQDCTLSLQLTTPANAPIGLYRLSLEASTGYQGSSFVLGHFILLFNAWCPADAVYL | |
| DSEEERQEYVLTQQGFIYQGSAKFIKNIPWNFGQFQDGILDICLILLDVNPKFLKNAGRDCSRRSSPVYVGRVGS | |
| GMVNCNDDQGVLLGRWDNNYGDGVSPMSWIGSVDILRRWKNHGCQRVKYGQCWVFAAVACTVLRCLGIPTRVVTN | |
| YNSAHDQNSNLLIEYFRNEFGEIQGDKSEMIWNFHCWVESWMTRPDLQPGYEGWQALDPTPQEKSEGTYCCGPVP | |
| VRAIKEGDLSTKYDAPFVFAEVNADVVDWIQQDDCSVHKSINRSLIVGLKISTKSVGRDEREDITHTYKYPEGSS | |
| EEREAFTRANHLNKLAEKEETGMAMRIRVGQSMNMGSDFDVFAHITNNTAEEYVCRLLLCARTVSYNGILGPECG | |
| TKYLLNLTLEPFSEKSVPLCILYEKYRDCLTESNLIKVRALLVEPVINSYLLAERDLYLENPEIKIRILGEPKQK | |
| RKLVAEVSLQNPLPVALEGCTFTVEGAGLTEEQKTVEIPDPVEAGEEVKVRMDLVPLHMGLHKLVVNFESDKLKA | |
| VKGFRNVIIGPA | |
| NM_012323: V-maf musculo aponeurotic fibrosarcoma oncogene homolog F (MAFF) | |
| MSVDPLSSKALKIKRELSENTPHLSDEALMGLSVRELNRHLRGLSAEEVTRLKQRRRTLK | Seq. ID No. 142 |
| NRGYAASCRVKRVCQKEELQKQKSELEREVDKLARENAAMRLELDALRGKCEALQGFARS | |
| VAAARGPATLVAPASVITIVKSTPGSGSGPAHGPDPAHGPASCS | |
| NM_001085: Serine or cysteine proteinase inhibitor clade A member 3 (SERPINA3) | |
| MERMLPLLALGLLAAGFCPAVLCHPNSPLDEENLTQENQDRGTHVDLGLASANVDFAFSL | Seq. ID No. 143 |
| YKQLVLKALDKNVIFSPLSISTALAFLSLGAHNTTLTEILKASSSPHGDLLRQKFTQSFQ | |
| HLRAPSISSSDELQLSMGNAMFVKEQLSLLDRFTEDAKRLYGSEAFATDFQDSAAAKKLI | |
| NDYVKNGTRGKITDLIKDPDSQTMMVLVNYIFFKAKWEMPFDPQDTHQSRFYLSKKKWVM | |
| VPMMSLHHLTIPYFRDEELSCTVVELKYTGNASALFILPDQDKMEEVEAMLLPETLKRWR | |
| DSLEFREIGELYLPKFSISRDYNLNDILLQLGIEEAFTSKADLSGITGARNLAVSQVVHK | |
| VVSDVFEEGTEASAATAVKITLLSALVETRTIVRFNRPFLMIIVPTDTQNIFFMSKVTNP | |
| SKPRACIKQWGSQ | |
| NM_001511: GRO1 oncogene melanoma growth stimulating activityalpha (GRO1) | |
| MARAALSAAPSNPRLLRVALLLLLLVAAGRRAAGASVATELRCQCLQTLQGIHPKNIQSV | Seq. ID No. 144 |
| NVKSPGPHCAQTEVIATLKNGRKACLNPASPIVKKIIEKMLNSDKSN | |
| NM_000591: CD14 antigen (CD14) | |
| MERASCLLLLLLPLVHVSATTPEPCELDDEDFRCVCNFSEPQPDWSEAFQCVSAVEVEIH | Seq. ID No. 145 |
| AGGLNLEPFLKRVDADADPRQYADTVKALRVRRLTVGAAQVPAQLLVGALRVLAYSRLKE | |
| LTLEDLKITGTMPPLPLEATGLALSSLRLRNVSWATGRSWLAELQQWLKPGLKVLSIAQA | |
| HSPAFSCEQVRAFPALTSLDLSDNPGLGERGLMAALCPHKFPAIQNLALRNTGMETPTGV | |
| CAALAAAGVQPHSLDLSHNSLRATVNPSAPRCMWSSALNSLNLSFAGLEQVPKGLPAKLR | |
| VLDLSCNRLNRAPQPDELPEVDNLTLDGNPFLVPGTALPHEGSMNSGVVPACARSTLSVG | |
| VSGTLVLLQGARGFA | |
| NM_015319: Tensin 2 (KIAA1075) | |
| MDGGGVCVGRGDLLSSPQALGQLLRKESRPRRAMKPRKAEPHSFREKVFRKKPPVCAVCK | Seq. ID No. 146 |
| VTIDGTGVSCRVCKVATHRKCEAKVTSACQALPPVELRRNTAPVRRIEHLGSTKSLNHSK | |
| QRSTLPRSFSLDPLMERRWDLDLTYVTERILAAAFPARPDEQRHRGHLRELAHVLQSKHR | |
| DKYLLFNLSEKRHDLTRLNPKVQDFGWPELHAPPLDKLCSICKAMETWLSADPQHVVVLY | |
| CKGNKGKLGVIVSAYMHYSKISAGADQALATLTMRKFCEDKVATELQPSQRRYISYFSGL | |
| LSGSIRMNSSPLFLHYVLIPMLPAFEPGTGFQPFLKIYQSMQLVYTSGVYHIAGPGPQQL | |
| CISLEPALLLKGDVMVTCYHKGGRGTDRTLVFRVQFHTCTIHGPQLTFPKDQLDEAWTDE | |
| RFPFQASVEFVFSSSPEKIKGSTPRNDPSVSVDYNTTEPAVRWDSYENFNQHHEDSVDGS | |
| LTHTRGPLDGSPYAQVQRPPRQTPPAPSPEPPPPPMLSVSSDSGHSSTLTTEPAAESPGR | |
| PPPTAAERQELDRLLGGCGVASGGRGAGRETAILDDEEQPTVGGGPHLGVYPGHRPGLSR | |
| HCSCRQGYREPCGVPNGGYYRPEGTLERRRLAYGGYEGSPQGYAEASMEKRRLCRSLSEG | |
| LYPYPPEMGKPATGDFGYRAPGYREVVILEDPGLPALYPCPACEEKLALPTAALYGLRLE | |
| REAGEGWASEAGKPLLHPVRPGHPLPLLLPACGHHHAPMPDYSCLKPPKAGEEGHEGCSY | |
| TMCPEGRYGHPGYPALVTYSYGGAVPSYCPAYGRVPHSCGSPGEGRGYPSPGAHSPRAGS | |
| ISPGSPPYPQSRKLSYEIPTEEGGDRYPLPGHLASAGPLASAESLEPVSWREGPSGHSTL | |
| PRSPRDAPCSASSELSGPSTPLHTSSPVQGKESTRRQDTRSPTSAPTQRLSPGEALPPVS | |
| QAGTGKAPELPSGSGPEPLAPSPVSPTFPPSSPSDWPQERSPGGHSDGASPRSPVPTTLP | |
| GLRHAPWQGPRGPPDSPDGSPLTPVPSQMPWLVASPEPPQSSPTPAFPLAASYDTNGLSQ | |
| PPLPEKRHLPGPGQQPGPWGPEQASSPARGISHHVTFAPLLSDNVPQTPEPPTQESQSNV | |
| KFVQDTSKFWYKPHLSRDQAIALLKDKDPGAFLIRDSHSFQGAYGLALKVATPPPSAQPW | |
| KGDPVEQLVRHFLIETGPKGVKIKGCPSEPYFGSLSALVSQHSISPISLPCCLRILSKDP | |
| LEETPEAPVPTNMSTAADLLRQGAACSVLYLTSVETESLTGPQAVARASSAALSCSPRPT | |
| PAVVHFKVSAQGITLTDNQRKLFFRRHYPVNSITFSSTDPQDRRWTNPDGTTSKIFGFVA | |
| KKPGSPWENVCHLFAELDPDQPAGAIVTFITKVLLGQRK | |
| NM_001276: Chitinase 3-like 1, cartilage glycoprotein-39 (CHI3L1) | |
| MGVKASQTGFVVLVLLQCCSAYKLVCYYTSWSQYREGDGSCFPDALDRFLCTHIIYSFAN | Seq. ID No. 147 |
| ISNDHIDTWEWNDVTLYGMLNTLKNRNPNLKTLLSVGGWNFGSQRFSKIASNTQSRRTFI | |
| KSVPPFLRTHGFDGLDLAWLYPGRRDKQHFTTLIKEMKAEFIKEAQPGKKQLLLSAALSA | |
| GKVTIDSSYDIAKISQHLDFISIMTYDFHGAWRGTTGHHSPLFRGQEDASPDRFSNTDYA | |
| VGYMLRLGAPASKLVMGIPTFGRSFTLASSETGVGAPISGPGIPGRFTKEAGTLAYYEIC | |
| DFLRGATVHRTLGQQVPYATKGNQWVGYDDQESVKSKVQYLKDRQLAGAMVWALDLDDFQ | |
| GSFCGQDLRFPLTNAIKDALAAT | |
| NM_004353: Serine or cysteine proteinase inhibitor clade H (SERPINH1) | |
| MRSLLLGTLCLLAVALAAEVKKPVEAAAPGTAEKLSSKATTLAEPSTGLAFSLYQAMAKD | Seq. ID No. 148 |
| QAVENILVSPVVVASSLGLVSLGGKATTASQAKAVLSAEQLRDEEVHAGLGELLRSLSNS | |
| TARNVTWKLGSRLYGPSSVSFADDFVRSSKQHYNCEHSKINFPDKRSALQSINEWAAQTT | |
| DGKLPEVTKDVERTDGALLVNAMFFKPHWDEKFHHKMVDNRGFMVTRSYTVGVTMMHRTG | |
| LYNYYDDEKEKLQLVEMPLAHKLSSLIILMPHHVEPLERLEKLLTKEQLKIWMGKMQKKA | |
| VAISLPKGVVEVTHDLQKHLAGLGLTEAIDKNKADLSRMSGKKDLYLASVFHATAFELDT | |
| DGNPFDQDIYGREELRSPKLFYADHPFIFLVRDTQSGSLLFIGRLVRLKGDKMRDEL | |
| NM_005952: Metallothionein 1X(MT1X) | |
| MDPNCSCSPVGSCACAGSCKCKECKCTSCKKSCCSCCPVGCAKCAQGCICKGTSDKCSCCA | Seq. ID No. 149 |
| XM_030707: KIAA0620 protein (KIAA0620) | |
| MAPRAAGGAPLSARAAAASPPPFQTPPRCPVPLLLLLLLGAARAGALEIQRRFPSPTPTN | Seq. ID No. 150 |
| NFALDGAAGTVYLAAVNRLYQLSGANLSLEAEAAVGPVPDSPLCHAPQLPQASCEHPRRL | |
| TDNYNKILQLDPGQGLVVVCGSIYQGFCQLRRRGNISAVAVRFPPAAPPAEPVTVFPSML | |
| NVAANHPNASTVGLVLPPAAGAGGSRLLVGATYTGYGSSFFPRNRSLEDHRFENTPEIAI | |
| RSLDTRGDLAKLFTFDLNPSDDNILKIKQGAKEQHKLGFVSAFLHPSDPPPGAQSYAYLA | |
| LNSEARAGDKESQARSLLARICLPHGAGGDAKKLTESYIQLGLQCAGGAGRGDLYSRLVS | |
| VFPARERLFAVFERPQGSPAARAAPAALCAFRFADVRAAIRAARTACFVEPAPDVVAVLD | |
| SVVQGTGPACERKLNIQLQPEQLDCGAAHLQHPLSILQPLKATPVFRAPGLTSVAVASVN | |
| NYTAVFLGTVNGRLLKINLNESMQVVSRRVVTVAYGEPVHHVMQFDPADSGYLYLMTSHQ | |
| MARVKVAACNVHSTCGDCVGAADAYCGWCALETRCTLQQDCTNSSQQHFWTSASEGPSRC | |
| PAMTVLPSEIDVRQEYPGMILQISGSLPSLSGMEMACDYGNNIRTVARVPGPAFGHQIAY | |
| CNLLPRDQFPPFPPNQDHVTVEMSVRVNGRNIVKANFTIYDCSRTAQVYPHTACTSCLSA | |
| QWPCFWCSQQHSGVSNQSRCEASPNPTSPQDCPRTLLSPLAPVPTGGSQNILVPLANTAF | |
| FQGAALECSFGLEEIFEAVWVNESVVRCDQVVLHTTRKSQVFPLSLQLKGRPARFLDSPE | |
| PMTVMVYNCAMGSPDCSQCLGREDLGHLCVWSDGCRLRGPLQPMAGTCPAPEIRAIEPLS | |
| GPLDGGTLLTIRGRNLGRRLSDVAHGVWIGGVACEPLPDRYTVSEEIVCVTGPAPGPLSG | |
| VVTVNASKEGKSRDRFSYVLPLVHSLEPTMGPKAGGTRITIHGNDLHVGSELQVLVNDTD | |
| PCTELMRTDTSIACTMPEGALPAPVPVCVRFERRGCVHGNLTFWYMQNPVITAISPRRSP | |
| VSGGRTITVAGERFHMVQNVSMAVHHIGREPTLCKVLNSTLITCPSPGALSNASAPVDFF | |
| INGRAYADEVAVAEELLDPEEAQRGSRFRLDYLPNPQFSTAKREKWIKHHPGEPLTLVIH | |
| KEQDSLGLQSHEYRVKIGQVSCDIQIVSDRIIHCSVNESLGAAVGQLPITIQVGNFNQTI | |
| ATLQLGGSETAIIVSIVICSVLLLLSVVALFVFCTKSRRAERYWQKTLLQMEEMESQIRE | |
| EIRKGFAELQTDMTDLTKELNRSQGIPFLEYKHFVTRTFFPKCSSLYEERYVLPSQTLNS | |
| QGSSQAQETHPLLGEWKIPESCRPNMEEGISLFSSLLNNKHFLIVFVHALEQQKDFAVRD | |
| RCSLASLLTIALHGKLEYYTSIMKELLVDLIDASAAKNPKLMLRRTESVVEKMLTNWMSI | |
| CMYSCLRETVGEPFFLLLCAIKQQINKGSIDAITGKARYTLSEEWLLRENIEAKPRNLNV | |
| SFQGCGMDSLSVRAMDTDTLTQVKEKILEAFCKNVPYSQWPRAEDVDLGGSPPAHRATSF | |
| GTWTTPQWWKTAARSLTRWPITRSLKVPPWP | |
| NM_003254: Tissue inhibitor of metalloproteinase 1 (TIMP1) | |
| MAPFEPLASGILLLLWLIAPSRACTCVPPHPQTAFCNSDLVIRAKFVGTPEVNQTTLYQR | Seq. ID No. 151 |
| YEIKMTKMYKGFQALGDAADIRFVYTPAMESVCGYFHRSHNRSEEFLIAGKLQDGLLHIT | |
| TCSFVAPWNSLSLAQRRGFTKTYTVGCEECTVFPCLSIPCKLQSGTHCLWTDQLLQGSEK | |
| GFQSRHLACLPREPGLCTWQSLRSQIA | |
| NM_006185: Nuclear mitotic apparatus protein 1 (NUMA1) | |
| MTLHATRGAALLSWVNSLHVADPVEAVLQLQDCSIFIKIIDRIHGTEEGQQILKQPVSER | Seq. ID No. 152 |
| LDFVCSFLQKNRKHPSSPECLVSAQKVLEGSELELAKMTMLLLYHSTMSSKSPRDWEQFE | |
| YKIQAELAVILKFVLDHEDGLNLNEDLENFLQKAPVPSTCSSTFPEELSPPSHQAKREIR | |
| FLELQKVASSSSGNNFLSGSPASPMGDILQTPQFQMRRLKKQLADERSNRDELELELAEN | |
| RKLLTEKDAQIAMMQQRIDRLALLNEKQAASPLEPKELEELRDKNESLTMRLHETLKQCQ | |
| DLKTEKSQMDRKINQLSEENGDLSFKLREFASHLQQLQDALNELTEEHSKATQEWLEKQA | |
| QLEKELSAALQDKKCLEEKNEILQGKLSQLEEHLSQLQDNPPQEKGEVLGDVLQLETLKQ | |
| EAATLAANNTQLQARVEMLETERGQQEAKLLAERGHFEEEKQQLSSLITDLQSSISNLSQ | |
| AKEELEQASQAHGARLTAQVASLTSELTTLNATIQQQDQELAGLKQQAKEKQAQLAQTLQ | |
| QQEQASQGLRHQVEQLSSSLKQKEQQLKEVAEKQEATRQDHAQQLATAAEEREASLRERD | |
| AALKQLEALEKEKAAKLEILQQQLQVANEARDSAQTSVTQAQREKAELSRKVEELQACVE | |
| TARQEQHEAQAQVAELELQLRSEQQKATEKERVAQEKDQLQEQLQALKESLKVTKGSLEE | |
| EKRRAADALEEQQRCISELKAETRSLVEQHKRERKELEEERAGRKGLEARLLQLGEAHQA | |
| ETEVLRRELAEAMAAQHTAESECEQLVKEVAAWRDGYEDSQQEEAQYGAMFQEQLMTLKE | |
| ECEKARQELQEAKEKVAGIESHSELQISRQQNKLAELHANLARALQQVQEKEVRAQKLAD | |
| DLSTLQEKMAATSKEVARLETLVRKAGEQQETASRELVKEPARAGDRQPEWLEEQQGRQF | |
| CSTQAALQAMEREAEQMGNELERLRAALMESQGQQQEERGQQEREVARLTQERGRAQADL | |
| ALEKAARAELEMRLQNALNEQRVEFATLQEALAHALTEKEGKDQELAKLRGLEAAQIKEL | |
| EELRQTVKQLKEQLAKKEKEHASGSGAQSEAAGRTEPTGPKLEALRAEVSKLEQQCQKQQ | |
| EQADSLERSLEAERASRAERDSALETLQGQLEEKAQELGHSQSALASAQRELAAFRTKVQ | |
| DHSKAEDEWKAQVARGRQEAERKNSLISSLEEEVSILNRQVLEKEGESKELKRLVMAESE | |
| KSQKLEESCACCRQRQPATVPELQNAALLCGRRCRASGREAEKQRVASENLRQELTSQAE | |
| RAEELGQELKAWQEKFFQKEQALSTLQLEHTSTQALVSELLPAKHLCQQLQAEQAAAEKR | |
| HREELEQSKQAAGGLRAELLRAQRELGELIPLRQKVAEQERTAQQLRAEKASYAEQLSML | |
| KKAHGLLAEENRGLGERANLGRQFLEVELDQAREKYVQELAAVRADAETRLAEVQREAQS | |
| TARELEVMTAKYEGAKVKVLEERQRFQEERQKLTAQVEELSKKLADSDQASKVQQQKLKA | |
| VQAQGGESQQEAQRFQAQLNELQAQLSQKEQAAEHYKLQMEKAKTHYDAKKQQNQELQEQ | |
| LRSLEQLQKENKELRAEAERLGHELQQAGLKTKEAEQTCRHLTAQVRSLEAQVAHADQQL | |
| RDLGKFQVATDALKSREPQAKPQLDLSIDSLDLSCEEGTPLSITSKLPRTQPDGTSVPGE | |
| PASPISQRLPPKVESLESLYFTPIPARSQAPLESSLDSLGDVFLDSGRKTRSARRRTTQI | |
| INITMTKKLDVEEPDSANSSFYSTRSAPASQASLRATSSTQSLARLGSPDYGNSALLSLP | |
| GYRPTTRSSARRSQAGVSSGAPPGRNSFYMGTCQDEPEQLDDWNRIAELQQRNRVCPPHL | |
| KTCYPLESRPSLSLGTITDEEMKTGDPQETLRRASMQPIQIAEGTGITTRQQRKRVSLEP | |
| HQGPGTPESKKATSCFPRPMTPRDRHEGRKQSTTEAQKKAAPASTKQADRRQSMAFSILN | |
| TPKKLGNSLLRRGASKKALSKASPNTRSGTRRSPRIATTTASAATAAAIGATPRAKGKAK | |
| H | |
| NM_004083: DNA-damage-inducible transcript 3 (DDIT3) | |
| MAAESLPFSFGTLSSWELEAWYEDLQEVLSSDENGGTYVSPPGNEEEESKIFTTLDPASL | Seq. ID No. 153 |
| AWLTEEEPEPAEVTSTSQSPHSPDSSQSSLAQEEEEEDQGRTRKRKQSGHSPARAGKQRM | |
| KEKEQENERKVAQLAEENERLKQEIERLTREVEATRRALIDRMVNLHQA | |
| NM_016272 Transducer of ERBB2 (TOB2) | |
| MQLEIKVALNFIISYLYNKLPRRRADLFGEELERLLKKKYEGHWYPEKPLKGSGFRCVHI | Seq. ID No. 154 |
| GEMVDPVVELAAKRSGLAVEDVRANVPEELSVWIDPFEVSYQIGEKGAVKVLYLDDSEGC | |
| GAPELDKEIKSSFNPDAQVFVPIGSQDSSLSNSPSPSFGQSPSPTFIPRSAQPITFTTAS | |
| FAATKFGSTKMKKGGGAASGGGVASSGAGGQQPPQQPRMARSPTNSLLKHKSLSLSMHSL | |
| NFITANPAPQSQLSPNAKEFVYNGGGSPSLFFDAADGQGSGTPGPFGGSGAGTCNSSSFD | |
| MAQVFGGGANSLFLEKTPFVEGLSYNLNTMQYPSQQFQPVVLAN | |

The foregoing description illustrates preferred embodiments of the present invention. It should be understood that those skilled in the art will envision modifications of the embodiments that are covered by the following claims.

1. A method of screening for schizophrenia in a population or of diagnosing schizophrenia in a host, comprising determining the magnitude of expression, in members of the population or in the host, of at least one gene selected from the group consisting of Small inducible cytokineA2, Growth arrest and DNA-damage-inducible beta, S100 calcium binding protein A8, Cyclin-dependent kinase inhibitor 1A p21/Cip1, Interleukin 1receptor-like 1, Transglutaminase, V-maf musculo aponeurotic fibrosarcoma oncogene homolog F, Serine or cysteine proteinase inhibitor clade A member 3, GRO1 oncogene melanoma growth stimulating activity alpha, CD14 antigen, Tensin 2, Chitinase 3-like 1, cartilage glycoprotein-39, Serine or cysteine proteinase inhibitor clade H, Metallothionein 1X, KIAA0620 protein, Tissue inhibitor of metalloproteinase 1, Nuclear mitotic apparatus protein 1, DNA-damage-inducibletranscript 3, and Transducer of ERBB2 in a sample and comparing the magnitude of expression to a baseline magnitude of expression of the gene, wherein increased gene expression indicates the presence of schizophrenia.

2. A method of screening for schizophrenia in a population or of diagnosing schizophrenia in a host according to claim 1, wherein the sample is taken from brain, spinal cord, lymphatic fluid, blood, urine or feces.

3. A method of screening for schizophrenia in a population or of diagnosing schizophrenia in a host according to claim 2, wherein the sample is taken from the anterior cingulate.

4. A method of screening for schizophrenia in a population or of diagnosing schizophrenia in a host according to claim 1, wherein the population is human.

5-8. (canceled)

9. A method for treating schizophrenia in a host, comprising lowering expression of at least one gene selected from the group consisting of Small inducible cytokineA2, Growth arrest and DNA-damage-inducible beta, S100 calcium binding protein A8, Cyclin-dependent kinase inhibitor 1A p21/Cip1, Interleukin 1receptor-like 1, Transglutaminase, V-maf musculo aponeurotic fibrosarcoma oncogene homolog F, Serine or cysteine proteinase inhibitor clade A member 3, GRO1 oncogene melanoma growth stimulating activity alpha, CD14 antigen, Tensin 2, Chitinase 3-like 1, cartilage glycoprotein-39, Serine or cysteine proteinase inhibitor clade H, Metallothionein 1X, KIAA0620 protein, Tissue inhibitor of metalloproteinase 1, Nuclear mitotic apparatus protein 1, DNA-damage-inducibletranscript 3, and Transducer of ERBB2 by administering to the host:
(a) an expression lowering amount of antisense oligonucleotide;
(b) an expression lowering amount of a ribozyme which cleaves RNA associated with expression of the gene;
(c) one or more nucleic acid molecules designed to promote triple helix formation with said at lease one gene or;
(d) one or more RNAi molecules designed to inhibit expression of said gene.

10. A method for treating schizophrenia in a host according to claim 9, wherein the host is human.

11-16. (canceled)

17. A method for treating schizophrenia in a host according to claim 31, wherein the antibody or functional antibody fragment is selected from the group consisting of whole antibody, humanized antibody, chimeric antibody, Fab fragment, Fab′ fragment, F(ab′)₂ fragment, single chain Fv fragment and diabody.

18. A transgenic nonhuman animal comprising stably integrated in its genome either a gene selected from the group consisting of Small inducible cytokineA2, Growth arrest and DNA-damage-inducible beta, S100 calcium binding protein A8, Cyclin-dependent kinase inhibitor 1A p21/Cip1, Interleukin 1receptor-like 1, Transglutaminase, V-maf musculo aponeurotic fibrosarcoma oncogene homolog F, Serine or cysteine proteinase inhibitor clade A member 3, GRO1 oncogene melanoma growth stimulating activity alpha, CD14 antigen, Tensin 2, Chitinase 3-like 1, cartilage glycoprotein-39, Serine or cysteine proteinase inhibitor clade H, Metallothionein 1X, KIAA0620 protein, Tissue inhibitor of metalloproteinase 1, Nuclear mitotic apparatus protein 1, DNA-damage-inducibletranscript 3, and Transducer of ERBB2, wherein expression of the gene is enhanced by one or more alterations in regulatory sequences of the gene such that the gene is expressed at higher than baseline levels and the animal exhibits schizophrenic behavior or an increased copy number of said gene, wherein said gene is expressed at higher than baseline levels and the animal exhibits schizophrenic behavior.

19. A transgenic nonhuman animal according to claim 18, wherein the transgenic nonhuman animal is a mammal.

20-21. (canceled)

22. A transgenic nonhuman animal according to claim 18, in which the expression of the gene is enhanced by one or more alterations in regulatory sequences of the gene, wherein the one or more alterations comprises substitution of a promoter having a higher rate of expression than the native promoter of the gene.

23. A transgenic nonhuman animal according to claim 22, wherein the promoter is an inducible promoter.

24. A transgenic nonhuman knockout animal whose genome comprises a homozygous disruption in one or more genes selected from the group consisting of Small inducible cytokineA2, Growth arrest and DNA-damage-inducible beta, S100 calcium binding protein A8, Cyclin-dependent kinase inhibitor 1A p21/Cip1, Interleukin 1receptor-like 1, Transglutaminase, V-maf musculo aponeurotic fibrosarcoma oncogene homolog F, Serine or cysteine proteinase inhibitor clade A member 3, GRO1 oncogene melanoma growth stimulating activity alpha, CD14 antigen, Tensin 2, Chitinase 3-like 1, cartilage glycoprotein-39, Serine or cysteine proteinase inhibitor clade H, Metallothionein 1X, KIAA0620 protein, Tissue inhibitor of metalloproteinase 1, Nuclear mitotic apparatus protein 1, DNA-damage-inducibletranscript 3, and Transducer of ERBB2, wherein said homozygous disruption prevents the expression of the gene, and wherein said homozygous disruption results in the transgenic knockout animal exhibiting decreased expression levels of the one or more genes as compared to a wild-type animal.

25. A method of screening for a therapeutic agent that modulates symptoms of schizophrenia comprising administering a candidate compound to a transgenic nonhuman animal according to claim 18 and determining the effect of the compound on symptoms associated with schizophrenia.

26. (canceled)

27. A method of screening for a therapeutic agent that modulates symptoms of schizophrenia comprising combining a candidate compound with a transgenic nonhuman animal according to claim 24 and determining the effect of the compound on symptoms associated with schizophrenia.

28. A method of screening for a compound useful in the treatment of schizophrenia comprising operatively linking a reporter gene which expresses a detectable protein to a regulatory sequence for a gene selected from the group consisting of Small inducible cytokineA2, Growth arrest and DNA-damage-inducible beta, S100 calcium binding protein A8, Cyclin-dependent kinase inhibitor 1A p21/Cip1, Interleukin 1receptor-like 1, Transglutaminase, V-maf musculo aponeurotic fibrosarcoma oncogene homolog F, Serine or cysteine proteinase inhibitor clade A member 3, GRO1 oncogene melanoma growth stimulating activity alpha, CD14 antigen, Tensin 2, Chitinase 3-like 1, cartilage glycoprotein-39, Serine or cysteine proteinase inhibitor clade H, Metallothionein 1X, KIAA0620 protein, Tissue inhibitor of metalloproteinase 1, Nuclear mitotic apparatus protein 1, DNA-damage-inducibletranscript 3, and Transducer of ERBB2 to produce a reporter construct; transfecting a cell with the reporter construct; exposing the transfected cell to a test compound; and comparing the level of expression of the reporter gene after exposure to the test compound to the level of expression before exposure to the test compound, wherein a lower level of expression after exposure is indicative of a compound useful for the treatment of schizophrenia.

29-30. (canceled)

31. A method for treating schizophrenia in a host, comprising reducing the amount of at least one protein encoded by the genes selected from the group consisting of Small inducible cytokineA2, Growth arrest and DNA-damage-inducible beta, S100 calcium binding protein A8, Cyclin-dependent kinase inhibitor 1A p21/Cip1, Interleukin 1receptor-like 1, Transglutaminase, V-maf musculo aponeurotic fibrosarcoma oncogene homolog F, Serine or cysteine proteinase inhibitor clade A member 3, GRO1 oncogene melanoma growth stimulating activity alpha, CD14 antigen, Tensin 2, Chitinase 3-like 1, cartilage glycoprotein-39, Serine or cysteine proteinase inhibitor clade H, Metallothionein 1X, KIAA0620 protein, Tissue inhibitor of metalloproteinase 1, Nuclear mitotic apparatus protein 1, DNA-damage-inducibletranscript 3, and Transducer of ERBB2 in a patient by administering an effective amount of antibody or functional antibody fragment sufficient to interfere with the normal activity of the protein.

32. A method for treating schizophrenia in a host according to claim 31, wherein the host is human.
